(19) United States
(12) Patent Application Publication
(10) Pub. No.: US 2024/0208894 A1
(43) Pub. Date: Jun. 27, 2024

(54) METHODS FOR PRODUCING OF LIPIDS

(71) Applicant: Pfizer Inc., New York, NY (US)

(72) Inventors: Nga My Do, Groton, CT (US); Shane Allen Eisenbeis, Pawcatuck, CT (US); Omar Abdelrahman Salman, Ann Arbor, MI (US)

(21) Appl. No.: 18/551,619

(22) PCT Filed: Apr. 6, 2022
(86) PCT No.: PCT/IB2022/053227

§ 371 Date: Sep. 21, 2023

Related U.S. Application Data
(60) Provisional application No. 63/324,162, filed on Mar. 28, 2022.
(60) Provisional application No. 63/216,895, filed on Jun. 30, 2021.
(60) Provisional application No. 63/173,335, filed on Apr. 9, 2021.

(51) Int. Cl. C07C 229/46; C07C 67/29; C07C 227/08; C07C 229/24

(52) U.S. Cl. CPC C07C 229/46; C07C 67/29; C07C 227/08; C07C 229/24

(57) ABSTRACT

The present disclosure provides methods for producing a compound having a chemical formula of Formula I, wherein R³ and R² are independently a i) linear or branched or cyclic, ii) saturated or unsaturated, and iii) substituted or unsubstituted hydrocarbon group comprising 1 to 30 carbon atoms; R³ is a i′) lincar or branched or cyclic. ii′) saturated or unsaturated, and iii′) substituted or unsubstituted hydrocarbon group: and L¹, L² and L³ independently are linkers.

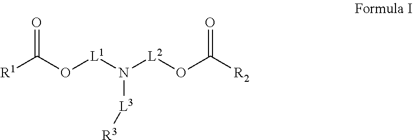

### CROSS REFERENCE TO RELATED APPLICATIONS

This application claims the priority to and the benefit of priority of U.S. Provisional Patent Application No. 63/173,335 filed Apr. 9, 2021, U.S. Provisional Patent Application No. 63/216,895 filed Jun. 30, 2021, and U.S. Provisional Patent Application No. 63/324,162 filed Mar. 28, 2022, the entire contents of which are hereby incorporated by reference in their entirety.

### FIELD OF THE INVENTION

The invention relates to methods for producing lipid compounds or intermediates thereof or pharmaceutically acceptable salts thereof. Some aspects relate to salts of the lipid compounds or intermediates thereof. The lipids and/or pharmaceutically acceptable salts thereof in combination with other lipids can be used for intracellular delivery of nucleic acids.

### BACKGROUND

Nucleic acid based therapeutics have enormous potential. Although free or naked nucleic acids can be used in some instances to transfect cells (Wolff et al. 1990, Science, 247, 1465-1468), it is generally advantageous or necessary to formulate the nucleic acid with at least a second agent that protects the nucleic acid from degradation during delivery, facilitates distribution to and in a target tissue, facilitates cellular uptake, and/or enables suitable intracellular processing. Free RNAs can be unstable, susceptible to nuclease digestion, and can have limited ability to gain access to the target tissue, cells, and/or intracellular compartments where the relevant translation machinery resides.

Lipids such as cationic lipids have been used for intracellular delivery of nucleic acids. Lipid containing nanoparticles containing encapsulated nucleic acids, are generally well-tolerated and can be used for targeted delivery of nucleic acids in a patient. For Example, U.S. Pat. No. 10,166,298 describes various cationic lipids, that can used for targeted delivery of various nucleic acids, such as messenger RNA (mRNA), antisense oligonucleotides, ribozymes, DNAzymes, plasmids, immune stimulating nucleic acids, antagomirs, anti-miRs, miRNA mimics, supermirs, and aptamers.

However, current methods for producing such lipids can be time consuming. For example, the lipid synthesis methods described in U.S. Pat. No. 10,166,298, can include steps that can run for multiple days in lab scale, and require isolation and purification of the reaction intermediates by chromatography. Although purification of the reaction intermediates by chromatography can increase purity of the final product, these purification steps can slow down the overall process, can increase costs, and can decrease overall process efficiency.

Thus, there remains a need for methods for relatively fast and cost effective preparation of lipids with high purity, such as lipids that can be used for nucleic acid delivery.

### SUMMARY

Applicant discloses solutions to at least some of the aforementioned problems associated with producing cationic lipids and intermediates for the production thereof. In one aspects, Applicant discloses producing cationic lipids and/or intermediates for the production thereof in a shorter amount of time than previously achieved. In some instances, the amount of time needed to produce the final product and/or intermediates for the production thereof is shortened in comparison due to one or more reaction steps using different reagents and/or reaction conditions than those used previously to produce a cationic lipid. In some instances, the amount of time needed to produce the final product and/or intermediates for the production thereof is shortened in comparison due to not needing to purify some or all of the lipid intermediates before proceeding with the next steps in the reaction process. In another aspect, Applicant discloses producing cationic lipids with high purity where the method does not involve isolation and purification of the lipid intermediates of the process by chromatography and/or using an isolated and/or purified lipid intermediate in downstream synthesis steps. As shown in a non-limiting manner in the Examples, a cationic lipid with purity excess of 97% and yield excess of 90% can be produced with the methods disclosed by the Applicant herein, wherein none of the process intermediates were purified by chromatography, such as silica gel column chromatography. Chemical reaction(s) in the one or more process steps of the methods disclosed by the Applicant herein may be monitored via chromatography and/or other analytical techniques; however, methods disclosed by the Applicant herein, in some embodiments, exclude using an isolated and/or purified intermediate, e.g., via column chromatography, in downstream steps. In another aspects, Applicant discloses salts of the cationic lipids and intermediates for the production thereof. In some instances, the salts are pharmaceutically acceptable, be environmentally safe, and/or have improved solubility or insolubility, bioavailability, purity, and/or steps for removal and/or replacement of the salt.

Those skilled in the art will recognize, or be able to ascertain using no more than routine experimentation, many equivalents to the specific embodiments described herein. Such equivalents are intended to be encompassed by the following Aspects.

Aspect 1 is directed to a method for producing a compound having a chemical formula of Formula I,

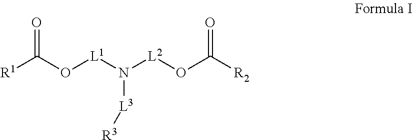

wherein R¹ and R² are independently a i) linear or branched or cyclic, ii) saturated or unsaturated, and iii) substituted or unsubstituted hydrocarbon group comprising 1 to 30 carbon atoms,
R³ is a i) linear or branched or cyclic, ii) saturated or unsaturated, and iii) substituted or unsubstituted hydrocarbon group,
L¹, L² and L³ are independently linkers, the method comprising:
   a) reacting a first acyl chloride having a chemical formula of R¹—(CO)—Cl with a first diol having a chemical formula of HO-L¹—CH₂—OH to form a first ester alcohol having a chemical formula of R¹—C(O)—O-L¹—CH₂—OH, and reacting a second acyl chloride having a chemical formula of R²-(CO)-Cl with a second diol having a chemical formula of HO-L¹—CH₂—OH to form a second ester alcohol having a chemical formula of R²—C(O)—O-L¹—CH₂—OH,
   b) oxidizing the first ester alcohol with a first oxidizing agent to form a first ester aldehyde having a chemical formula of R¹—C(O)—O-L¹-CHO, and oxidizing the second ester alcohol with a second oxidizing agent to form a second ester aldehyde having a chemical formula of R²—C(O)—O-L²-CHO; and
   c) reducing the first and second ester aldehyde in presence of a reducing agent and an amine having a chemical formula of R³-L³-NH₂, to form the compound of Formula I.

Aspect 2 is the method of aspect 1, wherein the first acyl chloride is formed by reacting a first fatty acid having a chemical formula of R¹—COOH with a first oxychloride, and the second acyl chloride is formed by reacting a second fatty acid having a chemical formula of R²-COOH with a second oxychloride, wherein the first and the second oxychloride are independently thionyl chloride, phosphoryl chloride, oxalyl chloride, or any combinations thereof.

Aspect 3 is the method of aspect 2, wherein the reaction conditions of the first fatty acid and first oxy chloride comprises contacting the first fatty acid and the first oxychloride at a molar ratio of 1:1 to 1:1.5, and the reaction conditions of the second fatty acid and second oxychloride comprises contacting the second fatty acid and the second oxychloride at a molar ratio of 1:1 to 1:1.5.

Aspect 4 is the method of any one of aspects 2 to 3, wherein a first fatty acid solution is contacted with a first oxychloride solution and a second fatty acid solution is contacted with a second oxychloride solution.

Aspect 5 is the method of aspect 4, wherein the first fatty acid solution comprises the first fatty acid and dichloromethane (DCM), the second fatty acid solution comprises the second fatty acid and DCM, the first oxychloride solution comprises the first oxychloride and DCM, and the second oxychloride solution comprises the second oxychloride and DCM.

Aspect 6 is the method of any one of aspects 2 to 5, wherein the first fatty acid and the first oxychloride are reacted at a temperature of 15° C. to 30° C., and the second fatty acid and the second oxychloride are reacted at a temperature of 15° C. to 30° C.

Aspect 7 is the method of any one of aspects 2 to 6, wherein the first fatty acid and the first oxychloride are reacted in presence of dimethylformamide (DMF), and the second fatty acid and the second oxychloride are reacted in presence of DMF.

Aspect 8 is the method of any one of aspects 2 to 7, wherein the first oxychloride, and/or the second oxychloride is oxalyl chloride.

Aspect 9 is the method of any one of aspects 1 to 8, wherein the first acyl chloride and the first diol are reacted in presence of a first tertiary amine, and the second acyl chloride and the second diol are reacted in presence of a second tertiary amine.

Aspect 10 is the method of aspect 9, wherein the first and/or second tertiary amine is triethylamine.

Aspect 11 is the method of any one of aspects 1 to 10, wherein the first acyl chloride and the first diol are contacted at a molar ratio of 0.8:3.5 to 1.2:2.5, and the second acyl chloride and the second diol are contacted at a molar ratio of 0.8:3.5 to 1.2:2.5.

Aspect 12 is the method of any one of aspects 1 to 11, wherein the first acyl chloride and the first diol are reacted at a temperature of 15° C. to 30° C., and the second acyl chloride and the second diol are reacted at a temperature of 15° C. to 30° C.

Aspect 13 is the method of any one of aspects 1 to 12, wherein the method further comprises,
adding a first base to a first esterification-product mixture comprising the first ester alcohol to form a first biphasic medium, said first biphasic medium comprises i) a first organic medium comprising the first ester alcohol, and ii) a first aqueous medium, and
adding a second base to a second esterification-product mixture comprising the second ester alcohol to form a second biphasic medium, said second biphasic medium comprises i) a second organic medium comprising the second ester alcohol, and ii) a second aqueous medium.

Aspect 14 is the method of aspect 13, further comprising,
i) separating the first organic medium from the first aqueous medium, and ii) washing the first organic medium with a first wash solution having a pH of 4 or below, and a second wash solution having a pH of 5 to 9, to form a washed first organic medium comprising the first ester alcohol, and
i′)separating the second organic medium from the second aqueous medium, and ii′) washing the second organic medium with a third wash solution having a pH of 4 or below, and a fourth wash solution having a of pH 5 to 9, to form a washed second organic medium comprising the second ester alcohol,
wherein the first ester alcohol in the washed first organic medium and the second ester alcohol in the washed second organic medium is oxidized in step b).

Aspect 15 is the method of any one of aspects 13 to 14, wherein the first base, and/or the second base is sodium hydroxide.

Aspect 16 is the method of any one of aspects 14 to 15, wherein the first and/or third wash solution comprise hydrogen chloride.

Aspect 17 is the method of any one of aspects 1 to 16, wherein the first oxidizing agent and/or the second oxidizing agent comprises sodium hypochlorite.

Aspect 18 is the method of aspect 17, wherein the sodium hypochlorite is sodium bicarbonate treated sodium hypochlorite.

Aspect 19 is the method of aspect 18, wherein the sodium bicarbonate treated sodium hypochlorite is formed by contacting sodium bicarbonate with sodium hypochlorite at a molar ratio of 0.2:1 to 0.5:1.

Aspect 20 is the method of any one of aspects 17 to 19, wherein the reaction conditions of the first ester alcohol and sodium hypochlorite comprises contacting the first ester alcohol and sodium hypochlorite at a molar ratio of 1:1 to 1:1.5, and the reaction conditions of the second ester alcohol and sodium hypochlorite comprises contacting the second ester alcohol and sodium hypochlorite at a molar ratio of 1:1 to 1:1.5.

Aspect 21 is the method of any one of aspects 1 to 20, wherein the oxidation of the first ester alcohol with the first oxidizing agent is catalyzed using a first oxidation catalyst, and the oxidation of the second ester alcohol with the second oxidizing agent is catalyzed using a second oxidation catalyst.

Aspect 22 is the method of aspect 21, wherein the first oxidation catalyst and/or second oxidation catalyst independently comprises potassium bromide and/or 2,2,6,6-tetramethylpyridine N-oxide (TEMPO).

Aspect 23 is the method of any one of aspects 1 to 22, wherein the first ester alcohol is oxidized at a temperature equal to or below 15° C., and the second ester alcohol is oxidized at a temperature equal to or below 15° C.

Aspect 24 is the method of any one of aspects 1 to 23, wherein the method further comprises,
washing a first oxidation-product mixture comprising the first ester aldehyde, and
washing a second oxidation-product mixture comprising the second ester aldehyde,
wherein the first ester aldehyde in the washed first oxidation-product mixture, and the second ester aldehyde in the washed second oxidation-product mixture is reduced in step (c).

Aspect 25 is the method of aspect 24, wherein i) the first oxidation-product mixture is washed with a first oxidation-wash solution having a pH of 4 or below, and a second oxidation-wash solution comprising sodium thiosulfate, and ii) the second oxidation-product mixture is washed with a third oxidation-wash solution having a pH of 4 or below, and a fourth oxidation-wash solution comprising sodium thiosulfate.

Aspect 26 is the method of aspect 25, wherein the first oxidation-wash solution and/or the third oxidation-wash solution comprises hydrochloric acid.

Aspect 27 is the method of any one of aspects 25 to 26, wherein the second oxidation-wash solution and the fourth oxidation-wash solution independently comprises 5 wt. % to 15 wt. % of sodium thiosulfate.

Aspect 28 is the method of any one of aspects 1 to 27, wherein in step (c) the first ester aldehyde and the second ester aldehyde is contacted with the amine at a total (first and second) ester aldehyde and amine molar ratio of 1:1 to 3:1.

Aspect 29 is the method of aspect 28, wherein the total ester aldehyde and amine molar ratio is 2:1 to 2.5:1.

Aspect 30 is the method of any one of aspects 1 to 29, wherein the reducing agent in step (c) comprises a hydride.

Aspect 31 is the method of aspect 30, wherein the hydride is sodium triacetoxyborohydride.

Aspect 32 is the method of aspect 31, wherein the first ester aldehyde and the second ester aldehyde is contacted with the sodium triacetoxyborohydride at a total (first and second) ester aldehyde and sodium triacetoxyborohydride molar ratio of 2:3 to 2:5.

Aspect 33 is the method of any one of aspects 30 to 32, wherein the first and second ester aldehydes are reduced with the hydride at a temperature of 30° C. or lower.

Aspect 34 is the method of any one of aspects 30 to 33, wherein the reduction of the first and second ester aldehydes with the amine and the hydride is quenched with a base.

Aspect 35 is the method of aspects 34, wherein 3 to 5 moles of the base per mole of ester aldehyde (total) reduced are used for quenching.

Aspect 36 is the method of any one of aspects 34 to 35, wherein the base is sodium hydroxide.

Aspect 37 is the method of any one of aspects 34 to 36, wherein an alkaline aqueous solution comprising the base is added to a reaction medium of the reduction reaction to quench the reduction reaction, and form a biphasic product mixture comprising an aqueous phase, and an organic phase comprising the compound of Formula I.

Aspect 38 is the method of aspect 37, further comprising adding an organic solvent to the biphasic product mixture.

Aspect 39 is the method of aspect 38, wherein the organic solvent comprises DCM.

Aspect 40 is the method of any one of aspects 1 to 29, wherein the reducing agent in step (c) comprises hydrogen (H2).

Aspect 41 is the method of aspect 40, wherein the reduction of the first ester aldehyde

and the second ester aldehyde with the amine and hydrogen is catalyzed with a metal catalyst.

Aspect 42 is the method of aspect 41, wherein the metal catalyst is platinum on carbon.

Aspect 43 is the method of any one of aspects 40 to 42, wherein the first ester aldehyde and the second ester aldehyde are reduced with hydrogen at a temperature of 25° C. to 45° C.

Aspect 44 is the method of any one of aspects 1 to 43, further comprising at least partially purifying the compound of Formula I by extraction, precipitation, silica gel chromatography, polymer resin chromatography, or a combination thereof.

Aspect 45 is the method of aspect 44, wherein the extraction purification comprises dissolving the compound of Formula I in an organic solvent to provide a solution of Formula I and extracting the solution of Formula I with an aqueous solution.

Aspect 46 is the method of aspect 45, wherein the organic solvent is n-heptane.

Aspect 47 is the method of any one of aspects 45 to 46, wherein the aqueous solution comprises a 10% aqueous methanol solution at a pH of 10-11.

Aspect 48 is the method of any one of aspects 44 to 47, wherein the silica gel chromatography purification comprises eluting the compound of Formula I through a silica gel chromatography column with an eluant comprising ethanol, isopropanol, n-heptane, ethyl acetate, or a mixture thereof.

Aspect 49 is the method of aspect 48, wherein the silica gel chromatography purification comprises eluting the compound of Formula I with an eluant mixture of n-heptane and ethyl acetate.

Aspect 50 is the method of aspect 49, wherein the silica gel chromatography purification comprises providing the eluant mixture of n-heptane and ethyl acetate in gradient form with increasing concentration of ethyl acetate.

Aspect 51 is the method of any one of aspects 1 to 50, further comprising purifying the compound of Formula I by distillation, the method comprising,
contacting the compound of Formula I formed in step (c) with n-heptane to form a n-heptane solution;

distilling the n-heptane solution at a temperature 30° C. to 45° C. and/or a pressure 0 to 0.3 bar to form a first distillation residue;

contacting the first distillation residue with ethanol to form an ethanol solution; and
distilling the ethanol solution at a temperature 30° C. to 45° C. and/or a pressure 0 to 0.3 bar to form a second distillation residue comprising compound of Formula I.

Aspect 52 is the method of aspect 51, wherein the second distillation residue comprises less than 5000 parts per million by weight (ppmw) of n-heptane and less than 50000 ppmw of ethanol.

Aspect 53 is the method of any one of aspects 1 to 52, wherein R¹ and R² are independently a branched, saturated, unsubstituted alkyl group comprising 1 to 30 carbons.

Aspect 54 is the method of any one of aspects 1 to 53, wherein R¹ and R² are the same.

Aspect 55 is the method of any one of aspects 1 to 54, wherein R¹ and R² both have the following structure

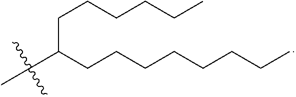 [SMILES: *C(CCCCCC)CCCCCCCC]

Aspect 56 is the method of any one of aspects 1 to 55, wherein R³ is a —CH₂OH group.

Aspect 57 is the method of any one of aspects 1 to 56, wherein L¹ has a chemical formula of —(CH₂)ₙ₁-X¹—(CH₂)ₙ₂—, wherein n1 and n2 are independently 0, 1, 2, 3, 4, 5, 6, 7, 8, 9, or 10, and X¹ is a linker.

Aspect 58 is the method of aspect 57, wherein X¹ is a bond, —HC═CH—, —C═C—, —C₆H₄—, —O—, or —S—.

Aspect 59 is the method of any one of aspects 1 to 56, wherein L¹ has a chemical formula of —(CH₂)ₙ—, where n is an integer from 2 to 15.

Aspect 60 is the method of any one of aspects 1 to 59, wherein L² has a chemical formula of —(CH₂)ₘ₁-X²—(CH₂)ₘ₂—, wherein m1 and m2 are independently 0, 1, 2, 3, 4, 5, 6, 7, 8, 9, or 10, and X² is a linker.

Aspect 61 is the method of aspect 53, wherein X² is a bond, —HC═CH—, —C═C—, —C₆H₄—, —O—, or —S—.

Aspect 62 is the method of any one of aspects 1 to 61, wherein L² has a chemical formula of —(CH₂)ₘ, where m is an integer from 2 to 15.

Aspect 63 is the method of any one of aspects 1 to 62, wherein L¹ and L² are the same.

Aspect 64 is the method of any one of aspects 1 to 63, wherein L¹ and L² both are —(CH₂)₅ —.

Aspect 65 is the method of any one of aspects 1 to 64, wherein L³ has a chemical formula of (CH₂)ₖ₁-X³—(CH₂)ₖ₂—, wherein k1 and k2 are independently 0, 1, 2, 3, 4, 5, 6, 7, 8, 9, or 10, and X³ is a linker.

Aspect 66 is the method of aspect 65, wherein X³ is a bond, —HC═CH—, —C═C—, —C₆H₄—, —O—, or —S—.

Aspect 67 is the method of any one of aspects 1 to 66, wherein L³ has a chemical formula of —(CH₂)ₖ—, where k is an integer from 1 to 15.

Aspect 68 is the method of any one of aspects 1 to 67, wherein L³ is —(CH₂)₃—.

Aspect 69 is the method of any one of aspects 1 to 61, wherein R¹ and R² are the same, L¹ and L² are the same, the first acyl chloride and second acyl chloride are the same and are formed in the same reaction medium, the first diol and the second diol are the same, the first and second ester alcohol are the same and are formed in the same reaction medium, and the first and second ester aldehyde are the same and are formed in the same reaction medium.

Aspect 70 is the method of any one of aspects 1 to 69, wherein Formula I is Formula II

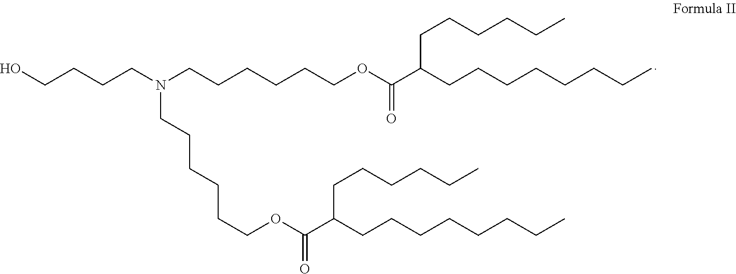 [SMILES: CCCCCCCCC(CCCCCC)C(=O)OCCCCCCN(CCCCO)CCCCCCOC(=O)C(CCCCCC)CCCCCCCC]

Aspect 71 is the method of any one of aspects 1 to 68, wherein i) R¹ and R² are different, and/or ii) L¹ and L² are different, and the first and second ester alcohols are formed separately, and the first and second ester aldehydes are formed separately.

Aspect 72 is the method of aspect 71, wherein the method optionally comprises separating the compound of Formula I, from other lipids formed by reduction of the first ester aldehyde and the second ester aldehyde with the amine.

Aspect 73 is a method for producing an acyl chloride having a chemical formula of R¹—(CO)—Cl, the method comprising reacting a fatty acid having a chemical formula of R¹—COOH with an oxychloride, wherein R¹ is a i) linear or branched or cyclic, ii) saturated or unsaturated, and iii) substituted or unsubstituted hydrocarbon group comprising 1 to 30 carbon atoms, and the oxychloride is thionyl chloride, phosphoryl chloride, oxalyl chloride, or any combinations thereof.

Aspect 74 is the method of aspect 73, wherein the reaction conditions of the fatty acid and oxy chloride comprises contacting the fatty acid and the oxychloride at a molar ratio of 1:1 to 1:1.5.

Aspect 75 is the method of any one of aspects 73 to 74, wherein a fatty acid solution is contacted with a oxychloride solution.

Aspect 76 is the method of aspect 75, wherein the fatty acid solution comprises the fatty acid and DCM, and the oxychloride solution comprises the oxychloride and DCM.

Aspect 77 is the method of any one of aspects 73 to 76, wherein the fatty acid and oxychloride is reacted at a temperature of 15° C. to 30° C.

Aspect 78 is the method of any one of aspects 73 to 77, wherein the fatty acid and oxychloride is reacted in presence of a catalyst comprising dimethylformamide (DMF).

Aspect 79 is the method of any one of aspects 73 to 78, wherein the oxychloride is oxalyl chloride.

Aspect 80 is the method of any one of aspects 73 to 79, wherein R¹ is a branched and saturated alkyl group comprising 1 to 30 carbons.

Aspect 81 is the method of any one of aspects 73 to 80, wherein R¹ has the following structure

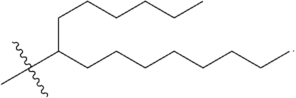 [SMILES: *C(CCCCCC)CCCCCCCC]

Aspect 82 is a method for producing an ester alcohol having a chemical formula of R¹—C(O)—O-L¹—CH₂—OH, the method comprising reacting an acyl chloride having a chemical formula of R¹—(CO)—Cl with a diol having a chemical formula of HO-L¹—CH₂—OH, wherein R¹ is a i) linear or branched or cyclic, ii) saturated or unsaturated, and iii) substituted or unsubstituted hydrocarbon group comprising 1 to 30 carbon atoms and L¹ is a linker.

Aspect 83 is the method of aspect 82, wherein the acyl chloride and the diol are reacted in presence of a tertiary amine.

Aspect 84 is the method of aspect 83, wherein the tertiary amine is triethylamine.

Aspect 85 is the method of any one of aspects 82 to 84, wherein the acyl chloride and the diol are contacted at a molar ratio of 0.8:3.5 to 1.2:2.5.

Aspect 86 is the method of any one of aspects 82 to 85, wherein the acyl chloride and the diol are reacted at a temperature of 15° C. to 30° C.

Aspect 87 is the method of any one of aspects 82 to 86, further comprising adding a base to a esterification-product mixture comprising the ester alcohol, to form a biphasic medium, said biphasic medium comprises i) an organic medium comprising the ester alcohol and ii) an aqueous medium.

Aspect 88 is the method of aspect 87, further comprising separating the organic medium from the aqueous medium, washing the organic medium with a first wash solution having a pH of 4 or below, and a second wash solution having a pH of 5 to 9.

Aspect 89 is the method of any one of aspects 87 to 88, wherein the base is sodium hydroxide.

Aspect 90 is the method of any one of aspects 88 to 89, wherein the first wash solution comprises hydrogen chloride.

Aspect 91 is the method of any one of aspects 82 to 90, wherein R¹ is a branched, saturated and unsubstituted alkyl group comprising 1 to 30 carbons.

Aspect 92 is the method of any one of aspects 82 to 91, wherein R¹ has the following structure

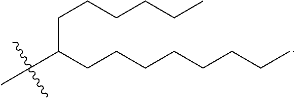 [SMILES: *C(CCCCCC)CCCCCCCC]

Aspect 93 is the method of any one of aspects 82 to 92, wherein L¹ has a chemical formula of —(CH₂)ₙ₁-X¹—(CH₂)₂—, wherein n1 and n2 are independently 0, 1, 2, 3, 4, 5, 6, 7, 8, 9, or 10, and X¹ is a linker.

Aspect 94 is the method of aspect 93, wherein X¹ is a bond, —HC═CH—, —C═C—, —C₆H₄—, —O—, or —S—.

Aspect 95 is the method of any one of aspects 82 to 94, wherein L¹ has a chemical formula of —(CH₂)ₙ—, where n is an integer from 2 to 15.

Aspect 96 is the method of any one of aspects 82 to 95, wherein the diol is 1, 6-hexane diol.

Aspect 97 is a method for producing an ester aldehyde having a chemical formula of R¹—C(O)—O-L¹-CHO, the method comprising oxidizing an ester alcohol having a chemical formula of R¹-C(O)-O- L¹—CH₂—OH with an oxidizing agent, wherein R¹ is a i) linear or branched or cyclic, ii) saturated or unsaturated, and iii) substituted or unsubstituted hydrocarbon group comprising 1 to 30 carbon atoms and L¹ is a linker.

Aspect 98 is the method of aspect 97, wherein the oxidizing agent comprises sodium hypochlorite.

Aspect 99 is the method of aspect 98, wherein the sodium hypochlorite is sodium bicarbonate treated sodium hypochlorite.

Aspect 100 is the method of aspect 99, wherein the sodium bicarbonate treated sodium hypochlorite is formed by contacting sodium bicarbonate with sodium hypochlorite at a molar ratio of 0.2:1 to 0.5:1.

Aspect 101 is the method of any one of aspects 98 to 100, wherein reaction conditions of the ester alcohol and the sodium hypochlorite comprises contacting the ester alcohol and the sodium hypochlorite at a molar ratio of 1:1 to 1:1.5.

Aspect 102 is the method of any one of aspects 97 to 101, wherein the oxidation of the ester alcohol with the oxidizing agent is catalyzed using an oxidation catalyst.

Aspect 103 is the method of aspect 102, wherein the oxidation catalyst comprises potassium bromide and/or 2,2,6,6-tetramethylpyridine N-oxide (TEMPO).

Aspect 104 is the method of any one of aspects 97 to 103, wherein the oxidation condition of the ester alcohol comprises a temperature of 15° C. or below.

Aspect 105 is the method of any one of aspects 97 to 104, wherein the method further comprises, washing a oxidation-product mixture solution comprising the ester aldehyde with an first oxidation-wash solution having a pH of 4 or below, and a second oxidation wash solution comprising sodium thiosulfate.

Aspect 106 is the method of aspect 105, wherein the first oxidation-wash solution comprises hydrochloric acid.

Aspect 107 is the method of any one of aspects 105 to 106, wherein the second oxidation wash solution comprises 5 wt. % to 15 wt. % of sodium thiosulfate.

Aspect 108 is the method of any one of aspects 97 to 107, wherein R¹ is a branched and

saturated alkyl group comprising 1 to 30 carbons.

Aspect 109 is the method of any one of aspects 97 to 108, wherein R¹ has the following structure

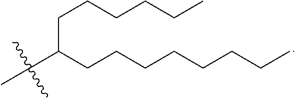 [SMILES: *C(CCCCCC)CCCCCCCC]

Aspect 110 is the method of any one of aspects 97 to 109, wherein L¹ has a chemical formula of —(CH₂)ₙ₁-X¹—(CH₂)₂—, wherein n1 and n2 are independently 0, 1, 2, 3, 4, 5, 6, 7, 8, 9, or 10, and X¹ is a linker.

Aspect 111 is the method of aspect 110, wherein X¹ is a bond, —HC═CH—, —C═C—, —C₆H₄—, —O—, or —S—.

Aspect 112 is the method of any one of aspects 97 to 111, wherein L¹ has a chemical formula of —(CH₂)ₙ—, where n is an integer from 2 to 15.

Aspect 113 is the method of any one of aspects 82 to 95, wherein L¹ is —(CH₂)₅—.

Aspect 114 is a method for producing a compound having the chemical formula of Formula I,

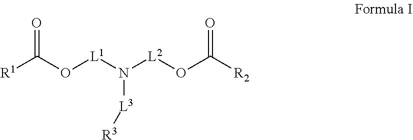

wherein R′ and R² are independently a i) linear or branched or cyclic, ii) saturated or unsaturated, and iii) substituted or unsubstituted hydrocarbon group comprising 1 to 30 carbon atoms,

R³ is a i′) linear or branched or cyclic, ii′) saturated or unsaturated, and iii′) substituted or unsubstituted hydrocarbon group,

L′, L² and L³ are independently linkers the method comprising:

reducing a first ester aldehyde having a chemical formula of R¹—C(O)—O-L¹-CHO and a second ester aldehyde having a chemical formula of R²—C(O)—O-L²-CHO in presence of an amine having a chemical formula of R³-L³-NH₂, and a reducing agent.

Aspect 115 is the method of aspect 114, wherein the first ester aldehyde and the second ester aldehyde is contacted with the amine at a total (first and second) ester aldehyde and amine molar ratio of 1:1 to 3:1.

Aspect 116 is the method of aspect 115, wherein the total ester aldehyde and amine molar ratio is 2:1 to 2.5:1.

Aspect 117 is the method of any one of aspects 114 to 116, wherein the reducing agent comprises a hydride.

Aspect 118 is the method of aspect 117, wherein the hydride is sodium triacetoxyborohydride.

Aspect 119 is the method of any one of aspects 114 to 118, wherein the first ester aldehyde and the second ester aldehyde is contacted with the sodium triacetoxyborohydride at a total (first and second) ester aldehyde and sodium triacetoxyborohydride molar ratio of 2:3 to 2:5.

Aspect 120 is the method of any one of aspects 117 to 119, wherein the first and second ester aldehydes are reduced with the hydride at a temperature of 30° C. or lower.

Aspect 121 is the method of any one of aspects 114 to 120, wherein the reduction of the first and second ester aldehydes with the amine and the hydride is quenched with a base.

Aspect 122 is the method of aspect 121, wherein 3 to 5 moles of the base per mole of ester aldehyde (total) reduced are used for quenching.

Aspect 123 is the method of any one of aspects 121 to 122, wherein the base is sodium hydroxide.

Aspect 124 is the method of any one of aspects 121 to 123, wherein an alkaline aqueous solution comprising the base is added to a reaction medium of the reduction reaction to quench the reduction reaction and form a biphasic product mixture comprising an aqueous phase, and an organic phase comprising the compound of Formula I.

Aspect 125 is the method of aspect 124, further comprising adding an organic solvent to the biphasic product mixture.

Aspect 126 is the method of aspect 125, wherein the organic solvent comprises DCM.

Aspect 127 is the method of any one of aspects 114 to 116, wherein the reducing agent comprises hydrogen (H₂).

Aspect 128 is the method of aspect 127, wherein the reduction of the first ester aldehyde and the second ester aldehyde with the amine and hydrogen is catalyzed with a metal catalyst.

Aspect 129 is the method of aspect 128, wherein the metal catalyst is platinum on carbon.

Aspect 130 is the method of any one of aspects 127 to 129, wherein the first ester aldehyde and the second ester aldehyde is reduced with hydrogen at a temperature of 25° C. to 45° C.

Aspect 131 is the method of any one of aspects 114 to 130, further comprising purifying the compound of Formula I by distillation, the method comprising,
contacting the compound of Formula I formed by the reduction of the first ester aldehyde and the second ester aldehyde, with n-heptane to form a n-heptane solution;
distilling the n-heptane solution at a temperature 30° C. to 45° C. and/or a pressure 0 to 0.3 bar to form a first distillation residue;
contacting the first distillation residue with ethanol to form an ethanol solution; and distilling the ethanol solution at a temperature 30° C. to 45° C. and/or a pressure 0 to 0.3 bar to form a second distillation residue comprising compound of Formula I.

Aspect 132 is the method of aspect 131, wherein the second distillation residue comprises less than 5000 parts per million by weight (ppmw) of n-heptane and less than 50000 ppmw of ethanol.

Aspect 133 is the method of any one of aspects 114 to 132, wherein R¹ and R² are independently a branched and saturated alkyl group comprising 1 to 30 carbons.

Aspect 134 is the method of any one of aspects 114 to 133, wherein R¹ and R² are independently a branched, saturated, unsubstituted alkyl group comprising 1 to 30 carbons.

Aspect 135 is the method of any one of aspects 114 to 134, wherein R¹ and R² are the same.

Aspect 136 is the method of any one of aspects 114 to 135, wherein R¹ and R² both have the following structure

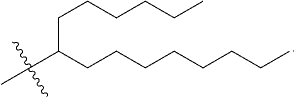 [SMILES: *C(CCCCCC)CCCCCCCC]

Aspect 137 is the method of any one of aspects 114 to 136, wherein R³ is a —CH₂OH group.

Aspect 138 is the method of any one of aspects 114 to 137, wherein L¹ has a chemical formula of (CH₂)ₙ₁ X¹ (CH₂)ₙ₂, wherein n1 and n2 are independently 0, 1, 2, 3, 4, 5, 6, 7, 8, 9, or 10, and X¹ is a linker.

Aspect 139 is the method of aspect 138, wherein X¹ is a bond, —HC═CH—, —C═C—, —C₆H₄—, —O—, or —S—.

Aspect 140 is the method of any one of aspects 114 to 139, wherein L¹ has a chemical formula of —(CH₂)ₙ—, where n is an integer from 2 to 15.

Aspect 141 is the method of any one of aspects 114 to 140, wherein L² has a chemical formula of —(CH₂)ₘ₁-X²—(CH₂)ₘ₂—, wherein m1 and m2 are independently 0, 1, 2, 3, 4, 5, 6, 7, 8, 9, or 10, and X² is a linker.

Aspect 142 is the method of aspect 141, wherein X² is a bond, —HC═CH—, —C═C—, —C₆H₄—, —O—, or —S—.

Aspect 143 is the method of any one of aspects 114 to 142, wherein L² has a chemical formula of —(CH₂)ₘ—, where m is an integer from 2 to 15.

Aspect 144 is the method of any one of aspects 114 to 143, wherein L¹ and L² are the same.

Aspect 145 is the method of any one of aspects 114 to 144, wherein L¹ and L² both are —(CH₂)₅—.

Aspect 146 is the method of any one of aspects 114 to 145, wherein L³ has a chemical formula of —(CH₂)ₖ₁-X³—(CH₂)ₖ₂—, wherein k1 and k2 are independently 0, 1, 2, 3, 4, 5, 6, 7, 8, 9, or 10, and X³ is a linker.

Aspect 147 is the method of aspect 146, wherein X³ is a bond, —HC═CH—, —C═C—, —C₆H₄—, —O—, or —S—.

Aspect 148 is the method of any one of aspects 114 to 147, wherein L³ has a chemical formula of —(CH₂)ₖ—, where k is an integer from 1 to 15.

Aspect 149 is the method of any one of aspects 114 to 148, wherein L³ is —(CH₂)₃—.

Aspect 150 is the method of any one of aspects 114 to 149, wherein Formula I is Formula II

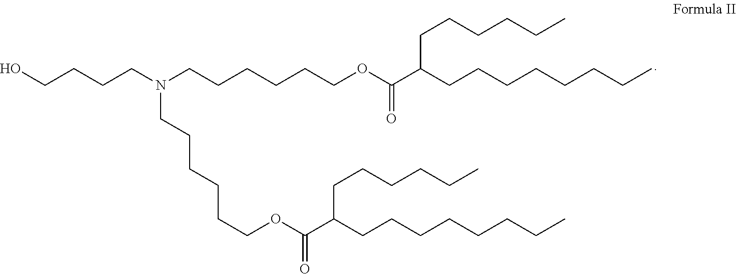 [SMILES: CCCCCCCCC(CCCCCC)C(=O)OCCCCCCN(CCCCO)CCCCCCOC(=O)C(CCCCCC)CCCCCCCC]

Aspect 151 is the method of any one of aspects 114 to 149, wherein i) R¹ and R² are different, and/or ii) L¹ and L² are different, and the method optionally comprises separating the compound of Formula I, from other lipids formed by reduction of the first ester aldehyde and the second ester aldehyde with the amine.

Aspect 152 is a method for producing a compound having the chemical formula of Formula Ia,

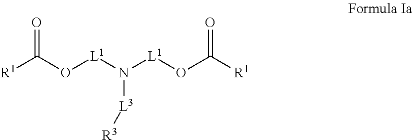

wherein R¹ is a i) linear or branched or cyclic, ii) saturated or unsaturated, and iii) substituted or unsubstituted hydrocarbon group comprising 1 to 30 carbon atoms,
R³ is a i′) linear or branched or cyclic, ii′) saturated or unsaturated, and iii′) substituted or unsubstituted hydrocarbon group,
L¹, and L³ are independently linkers the method comprising:
   a) reacting an acyl chloride having a chemical formula of R¹—(CO)—Cl with a diol having a chemical formula of HO-L¹—CH₂—OH to form an ester alcohol having a chemical formula of R¹—C(O)—O-L¹—CH₂—OH;
   b) oxidizing the ester alcohol with an oxidizing agent to form a ester aldehyde having a chemical formula of R¹—C(O)—O-L¹-CHO; and
   c) reducing the ester aldehyde in presence of a reducing agent and an amine having a chemical formula of R³-L³-NH₂, to form the compound of Formula Ia.

Aspect 153 is the method of aspect 152, wherein the acyl chloride is formed by reacting a fatty acid having a chemical formula of R¹—COOH with an oxychloride, wherein the oxychloride is thionyl chloride, phosphoryl chloride, oxalyl chloride, or any combinations thereof.

Aspect 154 is the method of aspect 153, wherein the reaction conditions of the fatty acid and oxy chloride comprises contacting the fatty acid and the oxychloride at a molar ratio of 1:1 to 1:1.5.

Aspect 155 is the method of any one of aspects 153 to 154, wherein a fatty acid solution is contacted with a oxychloride solution.

Aspect 156 is the method of aspect 155, wherein the fatty acid solution comprises the fatty acid and DCM, and the oxychloride solution comprises the oxychloride and DCM.

Aspect 157 is the method of any one of aspects 153 to 156, wherein the fatty acid and the oxychloride are reacted at a temperature of 15° C. to 30° C.

Aspect 158 is the method of any one of aspects 153 to 157, wherein the fatty acid and the oxychloride is reacted at in presence of a catalyst comprising DMF.

Aspect 159 is the method of any one of aspects 153 to 158, wherein the oxychloride is oxalyl chloride.

Aspect 160 is the method of any one of aspects 152 to 159, wherein the acyl chloride and the diol are reacted in presence of a tertiary amine.

Aspect 161 is the method of aspect 160, wherein the tertiary amine is triethylamine.

Aspect 162 is the method of any one of aspects 152 to 161, wherein the acyl chloride and the diol are reacted at a molar ratio of 0.8:3.5 to 1.2:2.5.

Aspect 163 is the method of any one of aspects 152 to 162, wherein the acyl chloride and the diol are reacted at a temperature of 15° C. to 30° C.

Aspect 164 is the method of any one of aspects 152 to 163, wherein the method further comprises, adding a base to an esterification-product mixture comprising the ester alcohol to form a biphasic medium, said biphasic medium comprises i) an organic medium comprising the ester alcohol and ii) an first aqueous medium.

Aspect 165 is the method of aspect 164, further comprising, separating the organic medium from the aqueous medium, washing the organic medium with a first wash solution having a pH 4 or below, and a second wash solution having a pH 5 to 9, wherein the ester alcohol in the washed organic medium is oxidized in step b).

Aspect 166 is the method of any one of aspects 164 to 165, wherein the base is sodium hydroxide.

Aspect 167 is the method of any one of aspects 165 to 166, wherein the first wash solution comprises hydrogen chloride.

Aspect 168 is the method of any one of aspects 152 to 167, wherein the oxidizing agent comprises sodium hypochlorite.

Aspect 169 is the method of aspect 168, wherein the sodium hypochlorite is sodium bicarbonate treated sodium hypochlorite.

Aspect 170 is the method of aspect 169, wherein the sodium bicarbonate treated sodium hypochlorite is formed by contacting sodium bicarbonate with sodium hypochlorite at a molar ratio of 0.2:1 to 0.5:1.

Aspect 171 is the method of any one of aspects 168 to 170, wherein reaction conditions of the ester alcohol and the sodium hypochlorite comprises contacting the ester alcohol and the sodium hypochlorite at a molar ratio of 1:1 to 1:1.5.

Aspect 172 is the method of any one of aspects 152 to 171, wherein the oxidation of the ester alcohol with the oxidizing agent is catalyzed using an oxidation catalyst.

Aspect 173 is the method of aspect 172, wherein the oxidation catalyst comprises potassium bromide and/or 2,2,6,6-tetramethylpyridine N-oxide (TEMPO).

Aspect 174 is the method of any one of aspects 152 to 173, wherein the ester alcohol is oxidized at a temperature equal to or below 15° C.

Aspect 175 is the method of any one of aspects 152 to 174, wherein the method further comprises, washing an oxidation-product mixture solution comprising the first ester aldehyde, wherein the ester aldehyde in the washed oxidation-product mixture solution is reduced in step (c).

Aspect 176 is the method of aspect 175, wherein the oxidation-product mixture solution is washed with a first oxidation-wash solution having a pH 4 or below, and a second oxidation-wash solution comprising sodium thiosulfate.

Aspect 177 is the method of aspect 176, wherein the first oxidation-wash solution and/or the third oxidation-wash solution comprises hydrochloric acid.

Aspect 178 is the method of any one of aspects 176 to 177, wherein the second oxidation-wash solution comprises 5 wt. % to 15 wt. % of sodium thiosulfate.

Aspect 179 is the method of any one of aspects 152 to 178, wherein in step (c) the ester aldehyde is contacted with the amine at a molar ratio of 1:1 to 3:1.

Aspect 180 is the method of aspect 179, wherein the ester aldehyde and amine molar ratio is 2:1 to 2.5:1.

Aspect 181 is the method of any one of aspects 152 to 180, wherein the reducing agent in step (c) comprises a hydride.

Aspect 182 is the method of aspect 181, wherein the hydride is sodium triacetoxyborohydride.

Aspect 183 is the method of any one of aspects 181 to 182, wherein the ester aldehyde is contacted with the sodium triacetoxyborohydride at a molar ratio of 2:3 to 2:5.

Aspect 184 is the method of any one of aspects 181 to 183, wherein the ester aldehyde are reduced with the hydride at a temperature of 30° C. or lower.

Aspect 185 is the method of any one of aspects 181 to 184, wherein the reduction of the ester aldehyde with the amine and the hydride is quenched with a base.

Aspect 186 is the method of aspects 185, wherein 3 to 5 moles of the base per mole of ester aldehyde reduced are used for quenching.

Aspect 187 is the method of any one of aspects 185 to 186, wherein the base in sodium hydroxide.

Aspect 188 is the method of any one of aspects 185 to 187, wherein an alkaline aqueous solution comprising the base is added to reaction medium of the reduction reaction to quench the reduction reaction and form a biphasic product mixture comprising an aqueous phase, and an organic phase comprising the compound of Formula Ia.

Aspect 189 is the method of aspect 188, further comprising adding an organic solvent to the biphasic product mixture.

Aspect 190 is the method of aspect 189, wherein the organic solvent comprises DCM.

Aspect 191 is the method of any one of aspects 152 to 180, wherein the reducing agent in step (c) comprises hydrogen (H2).

Aspect 192 is the method of aspect 191, wherein the reduction of the ester aldehyde with the amine and hydrogen is catalyzed with a metal catalyst.

Aspect 193 is the method of aspect 192, wherein the metal catalyst is platinum on carbon.

Aspect 194 is the method of any one of aspects 191 to 193, wherein the ester aldehyde is reduced with hydrogen at a temperature of 25° C. to 45° C.

Aspect 195 is the method of any one of aspects 152 to 194, further comprising purifying the compound of Formula Ia by distillation, the method comprising, contacting the compound of Formula Ia formed in step (c) with n-heptane to form a n-heptane solution;

distilling the n-heptane solution at a temperature 30° C. to 45° C. and/or a pressure 0 to 0.3 bar to form a first distillation residue;

contacting the first distillation residue with ethanol to form an ethanol solution; and distilling the ethanol solution at a temperature 30° C. to 45° C. and/or a pressure 0 to 0.3 bar to form a second distillation residue comprising compound of Formula Ia.

Aspect 196 is the method of aspect 195, wherein the second distillation residue comprises less than 5000 parts per million by weight (ppmw) of n-heptane and less than 50000 ppmw of ethanol.

Aspect 197 is the method of any one of aspects 195 to 196, wherein the second distillation residue comprises 95 wt. % or more of the compound of Formula Ia.

Aspect 198 is the method of any one of aspects 152 to 197, wherein R¹ is a branched and saturated alkyl group comprising 1 to 30 carbons.

Aspect 199 is the method of any one of aspects 152 to 198, wherein R¹ has the following structure

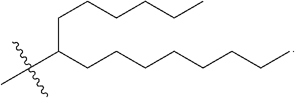 [SMILES: *C(CCCCCC)CCCCCCCC]

Aspect 200 is the method of any one of aspects 152 to 199, wherein R³ is a —CH₂OH group.

Aspect 201 is the method of any one of aspects 152 to 200, wherein L¹ has a chemical formula of —(CH₂)ₙ₁-X¹-(CH₂)ₙ₂—, wherein n1 and n2 are independently 0, 1, 2, 3, 4, 5, 6, 7, 8, 9, or 10, and X¹ is a linker.

Aspect 202 is the method of aspect 201, wherein X¹ is a bond, —HC═CH—, —C═C—, —(CH₂)₃—, —O—, or —S—.

Aspect 203 is the method of any one of aspects 152 to 202, wherein L¹ has a chemical formula of —(CH₂)ₙ—, where n is an integer from 2 to 15.

Aspect 204 is the method of any one of aspects 152 to 203, wherein L¹ is —(CH₂)₅—.

Aspect 205 is the method of any one of aspects 152 to 204, wherein L³ has a chemical formula of —(CH₂)ₖ₁-X³—(CH₂)ₖ₂—, wherein k1 and k2 are independently 0, 1, 2, 3, 4, 5, 6, 7, 8, 9, or 10, and X³ is a linker.

Aspect 206 is the method of aspect 205, wherein X³ is a bond, —HC═CH—, —C═C—, —(CH₂)₃—, —O—, or —S—.

Aspect 207 is the method of any one of aspects 152 to 206, wherein L³ has a chemical formula of —(CH₂)ₖ—, where k is an integer from 1 to 15.

Aspect 208 is the method of any one of aspects 152 to 207, wherein L³ is —(CH₂)₃—.

Aspect 209 is the method of any one of aspects 152 to 199, wherein Formula Ia is Formula II

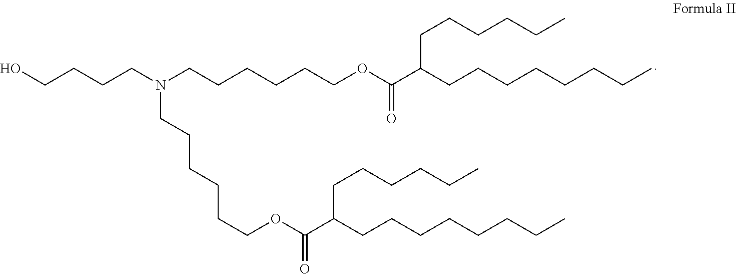 [SMILES: CCCCCCCCC(CCCCCC)C(=O)OCCCCCCN(CCCCO)CCCCCCOC(=O)C(CCCCCC)CCCCCCCC]

Aspect 210 is a salt having the chemical formula of Formula III:

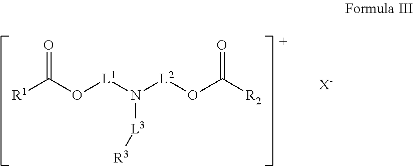

wherein R¹ and R² are independently a i) linear or branched or cyclic, ii) saturated or unsaturated, and iii) substituted or unsubstituted hydrocarbon group comprising 1 to 30 carbon atoms,
R³ is a i′) linear or branched or cyclic, ii′) saturated or unsaturated, and iii′) substituted or unsubstituted hydrocarbon group,
L¹, L² and L³ are independently linkers, and
X⁻ is chloride, bromide, iodide, sulfate, acetate, mesylate, tosylate, (1R)-(—)-10-camphorsulfonate, 1,2-ethanedisulfonate, oxalate, dibenzoyl-L-tartarate, phosphate, L-tartarate, maleate, fumarate, succinate, or malonate.

Aspect 211 is the salt of aspect 210, wherein R¹ and R² are independently a branched, saturated, unsubstituted alkyl group comprising 1 to 30 carbons.

Aspect 212 is the salt of any one of aspects 210 to 211, wherein R¹ and R² are the same.

Aspect 213 is the salt of any one of aspects 210 to 212, wherein R¹ and R² both have the following structure

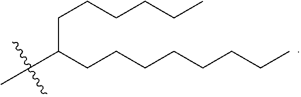 [SMILES: *C(CCCCCC)CCCCCCCC]

Aspect 214 is the salt of any one of aspects 210 to 213, wherein R³ is a —CH₂OH group.

Aspect 215 is the salt of any one of aspects 210 to 214, wherein L¹ has a chemical formula of —(CH₂)ₙ₁-X¹—(CH₂)ₙ₂—, wherein n1 and n2 are independently 0, 1, 2, 3, 4, 5, 6, 7, 8, 9, or 10, and X¹ is a linker.

Aspect 216 is the salt of aspect 215, wherein X¹ is a bond, —HC═CH—, —C═C—, —C₆H₄—, —O—, or —S—.

Aspect 217 is the salt of any one of aspects 210 to 216, wherein L¹ has a chemical formula of —(CH₂)ₙ—, where n is an integer from 2 to 15.

Aspect 218 is the salt of any one of aspects 210 to 217, wherein L² has a chemical formula of (CH₂)m1-X²—(CH₂)ₘ₂—, wherein m1 and m2 are independently 0, 1, 2, 3, 4, 5, 6, 7, 8, 9, or 10, and X² is a linker.

Aspect 219 is the salt of aspect 218, wherein X² is a bond, —HC═CH—, —C═C—, —C₆H₄—, —O—, or —S—.

Aspect 220 is the salt of any one of aspects 210 to 219, wherein L² has a chemical formula of
—(CH₂)ₘ—, where m is an integer from 2 to 15.

Aspect 221 is the salt of any one of aspects 210 to 220, wherein L¹ and L² are the same.

Aspect 222 is the salt of any one of aspects 210 to 221, wherein L¹ and L² both are —(CH₂)₅—.

Aspect 223 is the salt of any one of aspects 210 to 222, wherein L³ has a chemical formula of —(CH₂)ₖ₁-X³—(CH₂)ₖ₂—, wherein k1 and k2 are independently 0, 1, 2, 3, 4, 5, 6, 7, 8, 9, or 10, and X³ is a linker.

Aspect 224 is the salt of aspect 223, wherein X³ is a bond, —HC═CH—, —C═C—, —C₆H₄—, —O—, or —S—.

Aspect 225 is the salt of any one of aspects 210 to 224, wherein L³ has a chemical formula of
—(CH₂)ₖ—, where k is an integer from 1 to 15.

Aspect 226 is the salt of any one of aspects 210 to 225, wherein L³ is —(CH₂)₃—.

Aspect 227 is the salt of any one of aspects 210 to 226, wherein i) R¹ and R² are different, and/or ii) L¹ and L² are different.

Aspect 228 is the salt of any one of aspects 210 to 227, wherein the salt is in a crystallized form.

Aspect 229 is a method for forming a salt of any one of aspects 210 to 228 the method comprising contacting the compound of Formula I with an acid having a chemical formula of HX.

Aspect 230 is a salt having the chemical formula of Formula IV:

([R¹—C(O)—O-L¹—CH₂—O]⁻)ₓM^{x+} Formula IV
wherein R¹ is a i) linear or branched, ii) saturated or unsaturated, and iii) substituted or unsubstituted hydrocarbon group containing 1 to 30 carbon atoms,
L¹ is a linker,
x is 1 or 2, and
M^{x+} is cation selected from Na⁺, K⁺, Ca²⁺ and Mg²⁺.

Aspect 231 is the salt of aspect 230, wherein R¹ is a branched and saturated alkyl group comprising 1 to 30 carbons.

Aspect 232 is the salt of any one of aspects 230 to 231, wherein R¹ have the following structure

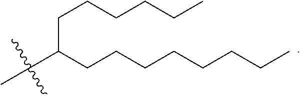 [SMILES: *C(CCCCCC)CCCCCCCC]

Aspect 233 is the salt of any one of aspects 230 to 232, wherein L¹ has a chemical formula of —(CH₂)ₙ₁-X¹—(CH₂)ₙ₂—, wherein n1 and n2 are independently 0, 1, 2, 3, 4, 5, 6, 7, 8, 9, or 10, and X¹ is a linker.

Aspect 234 is the salt of aspect 233, wherein X¹ is a bond, —HC═CH—, —C═C—, —C₆H₄—, —O—, or —S—.

Aspect 235 is the salt of any one of aspects 230 to 234, wherein L¹ has a chemical formula of
—(CH₂)ₙ—, where n is an integer from 2 to 15.

Aspect 236 is the salt of any one of aspects 230 to 235, wherein L¹ has a chemical formula of
—(CH₂)₅—.

Aspect 237 is the salt of any one of aspects 230 to 236, wherein the salt is in a crystallized form.

Aspect 238 is a method for forming a salt of any one of aspects 230 to 237, the method comprising contacting a compound having a chemical formula of R¹—C(O)—O-L¹—CH₂—OH with an base having a chemical formula of M(OH)ₓ, wherein M is a metal.

Aspect 239 is a compound having a chemical formula of Formula (48), (49), or (50), or a salt thereof,

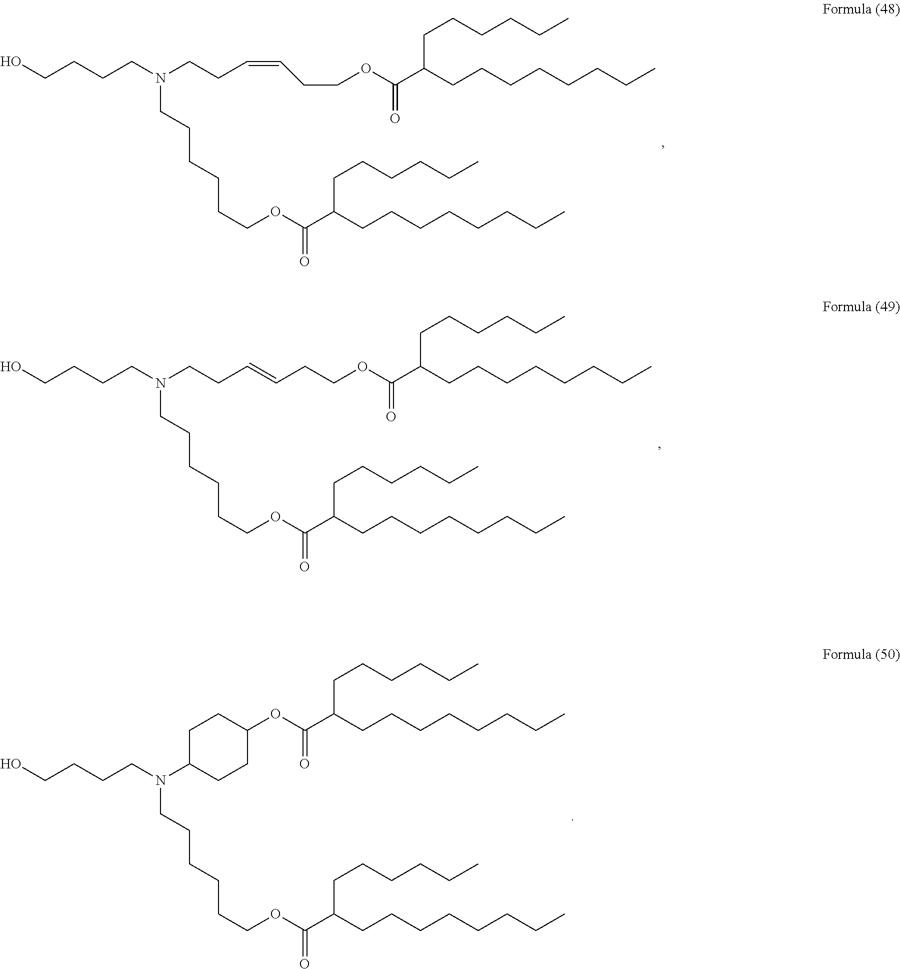 [SMILES: CCCCCCCCC(CCCCCC)C(=O)OCC/C=C\CCN(CCCCO)CCCCCCOC(=O)C(CCCCCC)CCCCCCCC ; CCCCCCCCC(CCCCCC)C(=O)OCC/C=C/CCN(CCCCO)CCCCCCOC(=O)C(CCCCCC)CCCCCCCC ; CCCCCCCCC(CCCCCC)C(=O)OCCCCCCN(CCCCO)C1CCC(OC(=O)C(CCCCCC)CCCCCCCC)CC1]

Aspect 240 is a method of making a compound of aspect 239, the method comprising:
a) reacting a first fatty acid with oxalyl chloride to form a first acyl chloride, and reacting a second fatty acid with oxalyl chloride to form a second acyl chloride;
b) reacting the first acyl chloride with a first diol to form a first ester alcohol, and reacting the second acyl chloride with a second diol to form a second ester alcohol;
c) oxidizing the first ester alcohol to form a first ester aldehyde, and oxidizing the second ester alcohol to form a second ester aldehyde; and
d) reducing the first and second ester aldehyde in presence of sodium triacetoxyborohydride and 4-amino-1-butanol to form the compound of Formula (48), (49), or (50),
   wherein the first and second fatty acid has the formula of

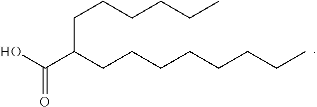 [SMILES: CCCCCCCCC(CCCCCC)C(=O)O]

Aspect 241 is a method of purifying a compound of Formula I,

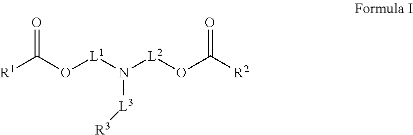

wherein R¹ and R² are independently a i) linear or branched or cyclic, ii) saturated or unsaturated, and iii) substituted or unsubstituted hydrocarbon group comprising 1 to 30 carbon atoms,
R³ is a i) linear or branched or cyclic, ii) saturated or unsaturated, and iii) substituted or unsubstituted hydrocarbon group,
L¹, L² and L³ are independently linkers,
the method comprising extracting, distilling, precipitating, purifying by chromatography, or a combination thereof.

Aspect 242 is the method of aspect 241, wherein the method comprises purifying by chromatography, wherein the chromatography is silica gel chromatography, polymer resin chromatography, or a combination thereof.

Aspect 243 is the method of any one of aspects 241 to 242, wherein the extraction purification comprises dissolving the compound of Formula I in an organic solvent to provide a solution of Formula I and extracting the solution of Formula I with an aqueous solution.

Aspect 244 is the method of aspect 243, wherein the organic solvent is n-heptane.

Aspect 245 is the method of any one of aspects 243 to 244, wherein the aqueous solution comprises a 10% aqueous methanol solution at a pH of 10-11.

Aspect 246 is the method of any one of aspects 242 to 245, wherein the silica gel chromatography purification comprises eluting the compound of Formula I through a silica gel chromatography column with an eluant comprising ethanol, isopropanol, n-heptane, ethyl acetate, or a mixture thereof.

Aspect 247 is the method of aspect 246, wherein the silica gel chromatography purification comprises eluting the compound of Formula I with an eluant mixture of n-heptane and ethyl acetate.

Aspect 248 is the method of aspect 247, wherein the silica gel chromatography purification comprises providing the eluant mixture of n-heptane and ethyl acetate in gradient form with increasing concentration of ethyl acetate.

Aspect 249 is the method of any one of aspects 241 to 248, wherein distilling comprises,
contacting the compound of Formula I with n-heptane to form a n-heptane solution;
distilling the n-heptane solution at a temperature 30° C. to 45° C. and/or a pressure 0 to 0.3 bar to form a first distillation residue;
contacting the first distillation residue with ethanol to form an ethanol solution; and
distilling the ethanol solution at a temperature 30° C. to 45° C. and/or a pressure 0 to 0.3 bar to form a second distillation residue comprising compound of Formula I.

Aspect 250 is the method of aspect 249, wherein the second distillation residue comprises less than 5000 parts per million by weight (ppmw) of n-heptane and less than 50000 ppmw of ethanol.

Aspect 251 is the method of any one of aspects 241 to 250, wherein R¹ and R² are independently a branched, saturated, unsubstituted alkyl group comprising 1 to 30 carbons.

Aspect 252 is the method of any one of aspects 241 to 251, wherein R¹ and R² are the same.

Aspect 253 is the method of any one of aspects 241 to 252, wherein R¹ and R² both have the following structure

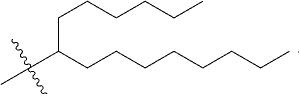 [SMILES: *C(CCCCCC)CCCCCCCC]

Aspect 254 is the method of any one of aspects 241 to 253, wherein R³ is a —CH₂OH group.

Aspect 255 is the method of any one of aspects 241 to 254, wherein L¹ has a chemical formula of (CH₂)ₙ₁-X¹—(CH₂)ₙ₂—, wherein n1 and n2 are independently 0, 1, 2, 3, 4, 5, 6, 7, 8, 9, or 10, and X¹ is a linker.

Aspect 256 is the method of aspect 255, wherein X¹ is a bond, —HC═CH—, —C═C—, —C₆H₄—, —O—, or —S—.

Aspect 257 is the method of any one of aspects 241 to 254, wherein L¹ has a chemical formula of (CH₂)ₙ—, where n is an integer from 2 to 15.

Aspect 258 is the method of any one of aspects 241 to 257, wherein L² has a chemical formula of —(CH₂)ₘ₁-X²—(CH₂)ₘ₂—, wherein m1 and m2 are independently 0, 1, 2, 3, 4, 5, 6, 7, 8, 9, or 10, and X² is a linker.

Aspect 259 is the method of aspect 258, wherein X² is a bond, —HC═CH—, —C═C—, —C₆H₄—, —O—, or —S—.

Aspect 260 is the method of any one of aspects 241 to 259, wherein L² has a chemical formula of —(CH₂)ₘ, where m is an integer from 2 to 15.

Aspect 261 is the method of any one of aspects 241 to 260, wherein L¹ and L² are the same.

Aspect 262 is the method of any one of aspects 241 to 261, wherein L¹ and L² both are —(CH₂)₅—.

Aspect 263 is the method of any one of aspects 241 to 262, wherein L³ has a chemical formula of (CH₂)k1-X³—(CH₂)ₖ₂—, wherein k1 and k2 are independently 0, 1, 2, 3, 4, 5, 6, 7, 8, 9, or 10, and X³ is a linker.

Aspect 264 is the method of aspect 263, wherein X³ is a bond, —HC═CH—, —C═C—, —C₆H₄—, —O—, or —S—.

Aspect 265 is the method of any one of aspects 241 to 264, wherein L³ has a chemical formula of (CH₂)ₖ—, where k is an integer from 1 to 15.

Aspect 266 is the method of any one of aspects 241 to 265, wherein L³ is —(CH₂)₃—.

Aspect 267 is the method of any one of aspects 241 to 266, wherein R¹ and R² are the same, L¹ and L² are the same.

Aspect 268 is the method of any one of aspects 241 to 267, wherein Formula I is Formula II

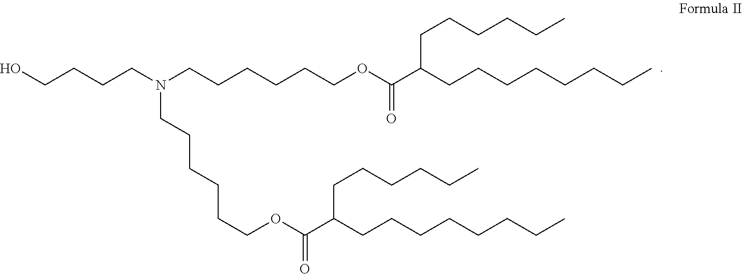 [SMILES: CCCCCCCCC(CCCCCC)C(=O)OCCCCCCN(CCCCO)CCCCCCOC(=O)C(CCCCCC)CCCCCCCC]

Aspect 269 is the method of any one of aspects 241 to 266, wherein i) R¹ and R² are different, and/or ii) L¹ and L² are different.

The following includes definitions of various terms and phrases used throughout this specification.

As used herein, the term “about,” or “approximately” is used to indicate that a value includes the standard deviation of error for the device or method being employed to determine the value. In some embodiments, the term “about” can be added to any numeral recited herein to the extent the numeral would have a standard deviation of error when measuring.

The terms “wt.%,” “vol. %,” or “mol. %” refers to a weight percentage of a component, a volume percentage of a component, or molar percentage of a component, respectively, based on the total weight, the total volume of material, or total moles, that includes the component. In a non-limiting example, 10 grams of component in 100 grams of the material is 10 wt. % of component.

The term “substantially” and its variations are defined to include ranges within 10%, within 5%, within 1%, or within 0.5%.

The terms “inhibiting” or “reducing” or “preventing” or “avoiding” or any variation of these terms, when used in the claims and/or the specification includes any measurable decrease or complete inhibition to achieve a desired result.

The term “effective,” as that term is used in the specification and/or claims, means adequate to accomplish a desired, expected, or intended result.

The use of the words “a” or “an” when used in conjunction with any of the terms “comprising,” “including,” “containing,” or “having” in the claims, or the specification, may mean “one,” but it is also consistent with the meaning of “one or more,” “at least one,” and “one or more than one.”

The phrase “and/or” means and or or. To illustrate, A, B, and/or C includes: A alone, B alone, C alone, a combination of A and B, a combination of A and C, a combination of B and C, or a combination of A, B, and C. In other words, “and/or” operates as an inclusive or.

The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The compositions, process, and systems disclosed by the Applicant herein can “comprise,” “consist essentially of,” or “consist of” particular ingredients, components, compositions, steps, etc. disclosed throughout the specification.

The term “hydrocarbon” as used herein refer to alkyl, heteroalkyl, cycloalkyl, aryl, heteroaryl groups. The groups (e.g., alkyl, heteroalkyl, cycloalkyl, aryl, and heteroaryl) can be substituted or unsubstituted, saturated or unsaturated, branched or unbranched, cyclic or acyclic.

The term “alkyl,” by itself or as part of another substituent, means, unless otherwise stated, a linear (i.e., unbranched) or branched carbon chain, which may be fully saturated, monounsaturated, or polyunsaturated. An unsaturated alkyl groups include those having one or more carbon-carbon double bonds (alkenyl) and those having one or more carbon-carbon triple bonds (alkynyl). The groups, —CH₃ (Me), —CH₂CH₃ (Et), —CH₂CH₂CH₃ (n-Pr), —CH(CH₃)₂ (iso-Pr), —CH₂CH₂CH₂CH₃ (n-Bu), —CH(CH₃)CH₂CH₃ (sec-butyl), —CH₂CH(CH₃)₂ (iso-butyl), —C(CH₃)₃ (tert-butyl), —CH₂C(CH₃)₃ (neo-pentyl), are all non-limiting examples of alkyl groups.

The term “heteroalkyl” or “substituted alkyl,” by itself or in combination with another term, means, unless otherwise stated, a linear or branched chain having at least one carbon atom and at least one heteroatom. The heteroatom in some instances is selected from the group consisting of one or more F, Cl, Br, I, O, N, S, P, and Si. In certain embodiments, the heteroatoms are selected from the group consisting of one or more O and N. The heteroatom(s) may be placed at any interior position, terminal of the heteroalkyl group or at the position at which the alkyl group is attached to the remainder of the molecule. Up to two heteroatoms may be consecutive. The following groups are all non-limiting examples of heteroalkyl groups: trifluoromethyl, —CH₂ F, —CH₂ Cl, —CH₂ Br, —CH₂ OH, —CH₂ OCH₃, —CH₂ OCH₂ CF₃, —CH₂OC(O)CH₃, —CH₂ NH₂, —CH₂ NHCH₃, —CH₂ N(CH₃)₂, —CH₂CH₂Cl, —CH₂CH₂OH, CH₂CH₂OC(O)CH₃, —CH₂CH₂ NHCO₂C(CH₃)₃, and —CH₂ Si(CH₃)₃. The heteroakyl group can be saturated or unsaturated.

The terms “cycloalkyl” and “heterocyclyl,” by themselves or in combination with other terms, means cyclic versions of “alkyl” and “heteroalkyl”, respectively. Additionally, for heterocyclyl, a heteroatom can occupy the position at which the heterocycle is attached to the remainder of the molecule.

The term “aryl” means a polyunsaturated, aromatic, hydrocarbon substituent. Aryl groups can be monocyclic or polycyclic (e.g., 2 to 3 or more rings that are fused together or linked covalently). The term “heteroaryl” refers to an aryl group that contains one to four heteroatoms selected from N, O, and S. A heteroaryl group can be attached to the remainder of the molecule through a carbon or heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below.

As described herein a “substituted” or a “substituted group” can refer to groups that include one or more substituents independently selected from: halogen, nitro, cyano, hydroxy, amino, mercapto, formyl, carboxy, oxo, carbamoyl, substituted or unsubstituted alkyl, substituted or unsubstituted heteroalkyl, alkoxy, alkylthio, alkylamino, (alkyl)zamino, alkylsulfinyl, alkylsulfonyl, arylsulfonyl, substituted or unsubstituted cycloalkyl, substituted or unsubstituted heterocyclyl, substituted or unsubstituted aryl, and substituted or unsubstituted heteroaryl. In certain aspects the substituents may be further substituted with one or more substituents independently selected from: halogen, nitro, cyano, hydroxy, amino, mercapto, formyl, carboxy, carbamoyl, unsubstituted alkyl, unsubstituted heteroalkyl, alkoxy, alkylthio, alkylamino, (alkyl)₂amino, alkylsulfinyl, alkylsulfonyl, arylsulfonyl, unsubstituted cycloalkyl, unsubstituted heterocyclyl, unsubstituted aryl, or unsubstituted heteroaryl. Exemplary substituents include, but are not limited to: —OH, oxo (═O), —Cl, —F, Br, C₁₋₄alkyl, phenyl, benzyl, —NH₂, —NH(C₁₋₄alkyl), —N(C₁₋₄-alkyl)₂, —NO₂, —S(C₁₋₄alkyl), —SO₂(C₁₋₄alkyl), —CO₂(C₁₋₄alkyl), and —O(C₁₋₄alkyl).

The term “alkoxy” means a group having the structure —OR′, where R′ is an optionally substituted alkyl or cycloalkyl group. The term “heteroalkoxy” similarly means a group having the structure —OR, where R is a heteroalkyl or heterocyclyl.

The term “amino” means a group having the structure —NR′R″, where R′ and R″ are independently hydrogen or an optionally substituted alkyl, heteroalkyl, cycloalkyl, or heterocyclyl group. The term “amino” includes primary, secondary, and tertiary amines.

The term “oxo” as used herein means an oxygen that is double bonded to a carbon atom.

The term “alkylsulfonyl” as used herein means a moiety having the formula —S(O₂)-R′, where R′ is an alkyl group. R′ may have a specified number of carbons (e.g., “C₁₋₄ alkylsulfonyl”).

As used herein, the term “nitro” means —NO₂; the term “halo” designates —F, —Cl, —Br or —I; the term “mercapto” means —SH; the term “cyano” means —CN; the term “azido” means —N₃; the term “silyl” means —SiH₃, and the term “hydroxyl” means —OH.

The term “pharmaceutically acceptable salts,” as used herein, refers to salts of compounds that are substantially non-toxic to living organisms. Typical pharmaceutically acceptable salts include those salts prepared by reaction of a compound with an inorganic or organic acid, or an organic base, depending on the substituents present on the compounds.

Non-limiting examples of inorganic acids which may be used to prepare pharmaceutically acceptable salts include or can exclude: hydrochloric acid, phosphoric acid, sulfuric acid, hydrobromic acid, hydroiodic acid, phosphorous acid and the like. Examples of organic acids which may be used to prepare pharmaceutically acceptable salts include or can exclude: aliphatic mono- and dicarboxylic acids, such as oxalic acid, carbonic acid, citric acid, succinic acid, phenyl- heteroatom-substituted alkanoic acids, aliphatic and aromatic sulfuric acids and the like. Pharmaceutically acceptable salts prepared from inorganic or organic acids thus include or can exclude hydrochloride, hydrobromide, nitrate, sulfate, pyrosulfate, bisulfate, sulfite, bisulfate, phosphate, monohydrogenphosphate, dihydrogenphosphate, metaphosphate, pyrophosphate, hydroiodide, hydro fluoride, acetate, propionate, formate, oxalate, citrate, lactate, p-toluenesulfonate, methanesulfonate, maleate, and the like.

Suitable pharmaceutically acceptable salts may also be formed by reacting compounds with an organic base such as methylamine, ethylamine, ethanolamine, lysine, ornithine and the like. Pharmaceutically acceptable salts include or can exclude the salts formed between carboxylate or sulfonate groups found on some of the compounds disclosed by the Applicant herein and inorganic cations, such as sodium, potassium, ammonium, or calcium, or such organic cations as isopropylammonium, trimethylammonium, tetramethylammonium, and imidazolium.

It should be recognized that the particular anion or cation forming a part of any salt of the compounds disclosed by the Applicant herein in some instances is not critical, so long as the salt, as a whole, is pharmacologically acceptable. However, in some instances, use of particular salts provides benefits, such as increased or decreased solubility in certain solvents or bioavailability, increased ability to remove or retain the anion or cation in downstream steps, increased safety for administration to a subject, decrease in environmentally dangerous waste, and/or increased environmental safety of the intermediates and/or final products.

Additional examples of pharmaceutically acceptable salts and their methods of preparation and use are presented in Handbook of Pharmaceutical Salts: Properties, Selection and Use (2002), which is incorporated herein by reference.

Other objects, features and advantages of the present invention will become apparent from the following figures, detailed description, and examples. It should be understood, however, that the figures, detailed description, and examples, while indicating specific embodiments of the invention, are given by way of illustration only and are not meant to be limiting. Additionally, it is contemplated that changes and modifications within the spirit and scope of the invention will become apparent to those skilled in the art from this detailed description. In further embodiments, features from specific embodiments may be combined with features from other embodiments. For example, features from one embodiment may be combined with features from any of the other embodiments. In further embodiments, additional features may be added to the specific embodiments described herein.

### DESCRIPTION

Methods for producing cationic lipids and intermediates for the production thereof are described. The method can include forming the cationic lipid via intermediate formation of an ester alcohol and an ester aldehyde or an ester alcohol and an ester ketone. In some instances, the amount of time needed to produce the final product and/or intermediates for the production thereof is shortened in comparison that previously achieved, due to one or more reaction steps using different reagents and/or reaction conditions than those used previously to produce a cationic lipid. In some instances, the amount of time needed to produce the final product and/or intermediates for the production thereof is shortened in comparison due to not needing to purify some or all of the lipid intermediates before proceeding with the next steps in the reaction process. In another aspect, a method for producing cationic lipids with high purity is disclosed where the method does not involve isolation and purification of the lipid intermediates of the process by chromatography and/or using an isolated and/or purified lipid intermediate in downstream synthesis steps. In another aspects, salts of the cationic lipids and intermediates for the production thereof are disclosed. In some instances, the salts are pharmaceutically acceptable, be environmentally safe, and/or have improved solubility or insolubility, bioavailability, purity, and/or steps for removal and/or replacement of the salt.

These and other non-limiting aspects of the present invention are discussed in further detail in the following sections.

### I. Compounds Having Chemical Formula of Formula I

Certain aspects are directed to methods for producing a compound having the chemical formula of Formula I. The compound of Formula I can form a cationic lipid.

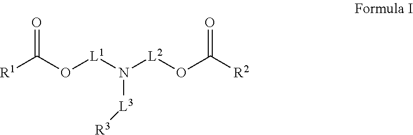

R¹ and R² can independently be a hydrocarbon group containing 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 carbon atoms. In certain aspects, R¹ and R² are independently a i) linear or branched or cyclic, ii) saturated or unsaturated, and iii) substituted or unsubstituted hydrocarbon group containing 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 carbon atoms. In certain aspects, R¹ and/or R² are independently a linear, saturated, substituted alkyl group. In certain aspects, R¹ and/or R² are independently a linear, saturated, unsubstituted alkyl group. In certain aspects, R¹ and/or R² are independently a linear, unsaturated, substituted alkyl group. In certain aspects, R¹ and/or R² are independently a linear, unsaturated, unsubstituted alkyl group. In certain aspects, R¹ and/or R² are independently a branched, saturated, substituted alkyl group. In certain aspects, R¹ and/or R² are independently a branched, saturated, unsubstituted alkyl group. In certain aspects, R¹ and/or R² are independently a branched, unsaturated, substituted alkyl group. In certain aspects, R¹ and/or R² are independently a branched, unsaturated, unsubstituted alkyl group. In certain aspects, R¹ and R² are independently a branched, saturated, unsubstituted alkyl group. In some particular aspects, R¹ and R² are independently a branched, saturated, unsubstituted alkyl group containing one or more branches containing independently 1, 2, 3, 4, 5, 6, 7, 8, 9, 10, 11, or 12 carbon atoms, wherein the alkyl group can contain (e.g., in total, in the branch(es) and in the backbone) 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 carbon atoms. In some particular aspects, R¹ and R² are independently a branched, saturated, unsubstituted alkyl group containing a branch containing 3, 4, 5, 6, 7, 8, 9, 10, 11, or 12 carbon atoms, and a backbone containing 7, 8, 9, 10, 11, 12, 13, 14, 15, 16, 17, or 18 carbon atoms, wherein the alkyl group can contain (e.g., in total, in the branch and in the backbone) 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 carbon atoms. In certain aspects, R′ and R² are the same. In certain aspects, R¹ and R² are different.

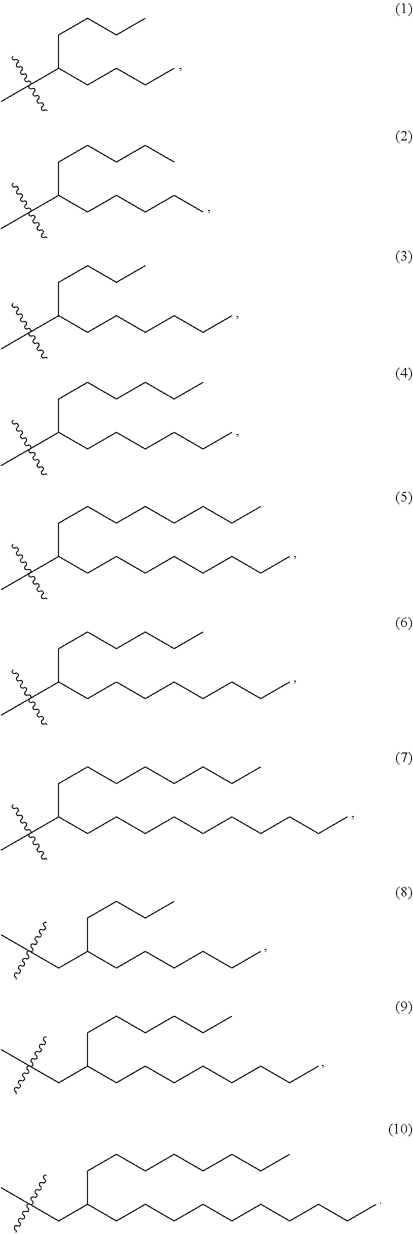

In certain aspects, R¹ and/or R² independently have the structure of any one of Formula (1) to (10). In certain aspects, R¹ and R² are the same, and each have the structure of any one of Formula (1) to (10). In certain aspects, R¹ and R² both have the structure of formula (6). In certain aspects, one or more R¹ and/or R² groups disclosed herein are excluded.

In certain aspects, L¹ has a chemical formula of (CH₂)ₙ₁-X¹ (CH₂)ₙ₂—, wherein n1 and n2 are independently 0, 1, 2, 3, 4, 5, 6, 7, 8, 9, or 10, and X¹ is a linker. In some aspects, X¹ is a bond, —HC═CH—, —C═C—, —C₆H₄—, —O—, or —S—. In some aspects, X¹ is —HC═CH—. The —HC—CH— of X¹ can be in E or Z configuration. In some aspects, X¹ is —C₆H₄—. In certain aspects, X¹ is a bond, the sum of n1 and n2 equals n, and L¹ has a chemical formula of -(CH₂)n -. In some aspects, n is 2, 3, 4, 5, 6, 7, 8, 9, 10, 11, 12, 13, 14, or 15.

In certain aspects, L² has a chemical formula of (CH₂)ₘ₁-X²—(CH₂)ₘ₂—, wherein ml and m2 are independently 0, 1, 2, 3, 4, 5, 6, 7, 8, 9. or 10, and X² is a linker. In some aspects, X² is a bond, —HC═CH—, —C═C—, —C₆H₄—, —O—, or —S—. In some aspects, X² is —HC═CH—. The —HC—CH— of X² can be in E or Z configuration. In some aspects, X² is —C₆H₄—. In certain aspects, X² is a bond, the sum of m1 and m2 equals m, and L² has a chemical formula of —(CH₂)ₘ—. In some aspects, m is 2, 3, 4, 5, 6, 7, 8, 9, 10, 11, 12, 13, 14, or 15.

In some aspects, L¹ and L² are the same. In some aspects, L¹ and L² are different. In some aspects, L¹ is —(CH₂)ₙ—, L² is —(CH₂)ₘ—, and n and m are the same. In some aspects, L¹ is —(CH₂)ₙ—, L² is —(CH₂)ₘ—, and n and m are different. In some particular aspects, L¹ and L² are both —(CH₂)₅—. In some aspects, L¹ is —(CH₂)ₘ₁—HC═CH—(CH₂)ₙ₂— and L² is —(CH₂)ₘ₁—HC—CH—(CH₂)ₘ₂—. In some aspects, L¹ is —(CH₂)ₙ₁—HC═CH—(CH₂)ₙ₂— and L² is —(CH₂)ₘ—. In some aspects, L¹ is —CH₂—HC═CH—(CH₂)₂— and L² is —(CH₂)₅—.

In some aspects, i) R¹ and R² are different, and ii) L¹ and L² are the same. In some aspects, i) R¹ and R² are the same, and ii) L¹ and L² are different. In some aspects, i) R¹ and R² are the same, and ii) L¹ and L² are the same. In some aspects, i) R¹ and R² are different, and ii) L¹ and L² are different.

R³ can be a i) substituted or unsubstituted, ii) linear, branched or cyclo hydrocarbon, and iii) saturated or unsaturated hydrocarbon group. In some aspects, R³ is a substituted alkyl group. In certain aspects, R³ contains 1, 2, 3, 4, 5, 6, 7, 8, 9, 10, 11, 12, 13, 14, 15, 16, 17, 18, 19, 20, 21, 22, 23, 24, or 25 carbon atoms. In certain aspects, R³ is a substituted alkyl group containing —OH, —OC(O)—R⁴, —C(O)OR⁵, —CN, —NC(O)R⁶, —OR⁷ substitution, wherein R⁴, R⁵, R⁶, and R⁷, are independently an alkyl group containing 1 to 5 carbons. In some particular aspects, R³ is a substituted alkyl group containing a terminal OH group. In certain aspects, R³ is —CH₂OH, —CHOHCH₂OH, —CH(CH₂CH₃)CH₂OH, —CHOHCH₂CH₃, —CH(CH₂OH)CHOH(CH₂)₁₄CH₃, —OC(O)CH₃, —C(O)OCH₂CH₃, —CN, —NC(O)CH₃, —OCH₃, or

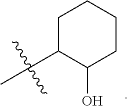 [SMILES: *C1CCCCC1O]

In certain aspects, L³ has a chemical formula of —(CH₂)ₖ₁-X³ (CH₂)ₖ₂—, wherein k1 and k2 are independently 0, 1, 2, 3, 4, 5, 6, 7, 8, 9. or 10, and X³ is a linker. In some aspects, X³ is a bond, —HC═CH—, —C═C—, —C₆H₄—, —O—, or —S—. In certain aspects, X³ is a bond, the sum of k1 and k2 equals k, and L³ has a chemical formula of —(CH₂)₂—. In some aspects, k is 0, 1, 2, 3, 4, 5, 6, 7, 8, 9, 10, 11, 12, 13, 14, or 15. In some aspects, k can be 0, and a direct bond between N (nitrogen) and R³ exists. In some particular aspects, k is 2, 3, 4, 5, 6, 7, 8, 9, 10, 11, or 12, and R³ is —CH₂OH group.

In some particular aspects, i) R¹ and R² are independently a branched, saturated, unsubstituted alkyl group; ii) L¹ is —(CH₂)ₙ—, and L² is —(CH₂)ₘ—, where n and m are independently 3, 4, 5, 6, 7, 8, 9, 10, 11, 12, 13, 14, or 15; iii) L³ is —(CH₂)ₖ—, where k is 2, 3, 4, 5, 6, 7, 8, 9, 10, 11, or 12; and iv) R³ is —CH₂OH group. In some particular aspects, i) R¹ and R² are the same and both are a branched, saturated, unsubstituted alkyl group; ii) L¹ is —(CH₂)ₙ—, and L² is —(CH₂)ₘ—, where n and m are the same, and both are 3, 4, 5, 6, 7, 8, 9, 10, 11, 12, 13, 14, or 15; iii) L³ is -(CH₂)ₖ—, where k is 2, 3, 4, 5, 6, 7, 8, 9, 10, 11, or 12; and iv) R³ is —CH₂OH group.

In some aspects, i) R¹ and R² are independently a branched, saturated, unsubstituted alkyl group; ii) L¹ is —(CH₂)ₙ₁-X¹—(CH₂)ₙ₂— where n1 and n2 are independently 0, 1, 2, 3, 4, 5, 6, 7, or 8, and X¹ is —HC═CH—, —C═C—, or —C₆H₄—, iii) L² is —(CH₂)ₘ—, where m is 3, 4, 5, 6, 7, 8, 9, 10, 11, 12, 13, 14, or 15; iv) L³ is —(CH₂)ₖ—, where k is 2, 3, 4, 5, 6, 7, 8, 9, 10, 11, or 12; and v) R³ is —CH₂OH group.

In certain aspects, the compounds of Formula I has the structure of any one of Formula (11) to (50)

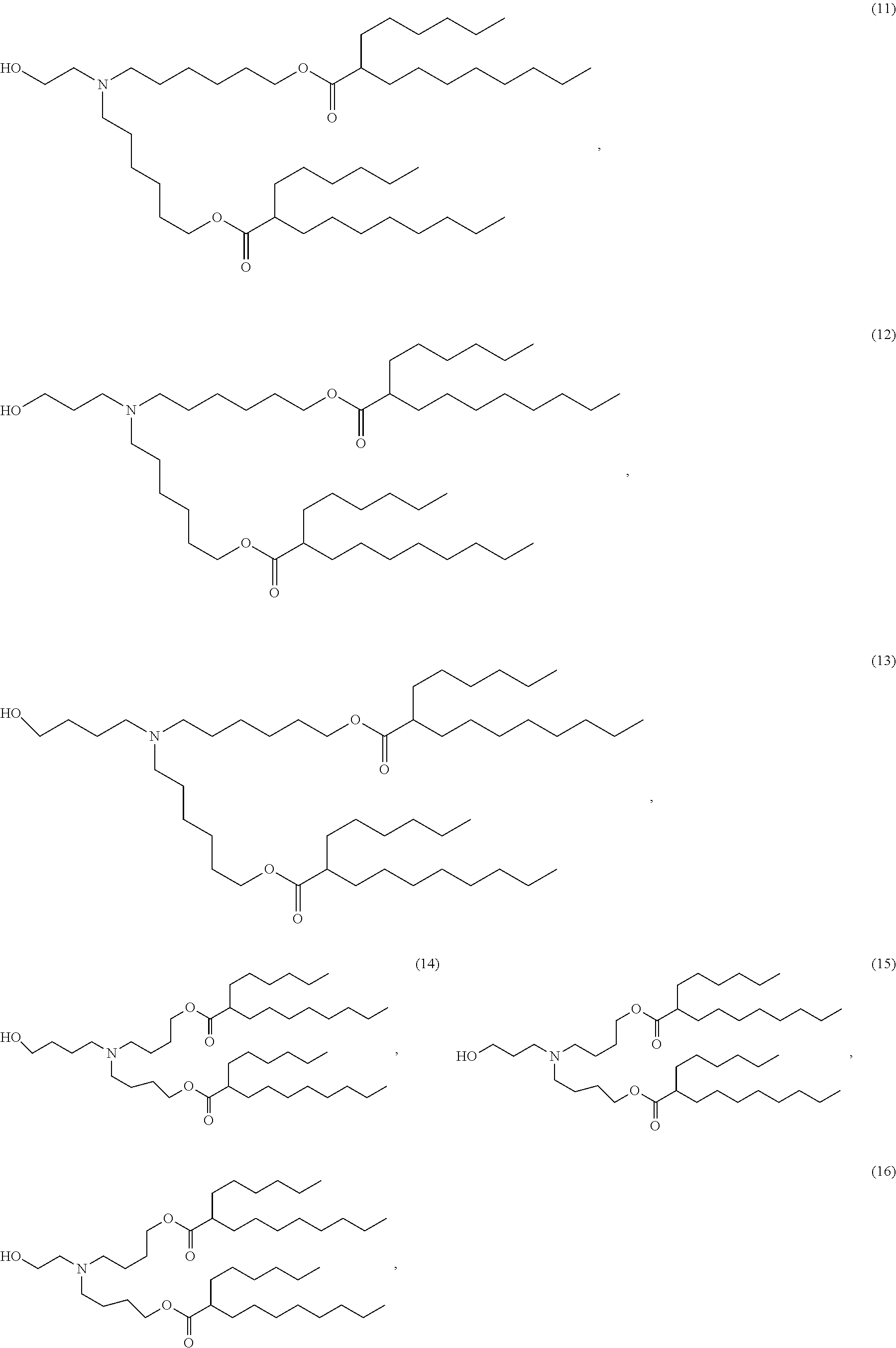 [SMILES: CCCCCCCCC(CCCCCC)C(=O)OCCCCCCN(CCO)CCCCCCOC(=O)C(CCCCCC)CCCCCCCC ; CCCCCCCCC(CCCCCC)C(=O)OCCCCCCN(CCCO)CCCCCCOC(=O)C(CCCCCC)CCCCCCCC ; CCCCCCCCC(CCCCCC)C(=O)OCCCCCCN(CCCCO)CCCCCCOC(=O)C(CCCCCC)CCCCCCCC ; CCCCCCCCC(CCCCCC)C(=O)OCCCCN(CCCCO)CCCCOC(=O)C(CCCCCC)CCCCCCCC ; CCCCCCCCC(CCCCCC)C(=O)OCCCCN(CCCO)CCCCOC(=O)C(CCCCCC)CCCCCCCC ; CCCCCCCCC(CCCCCC)C(=O)OCCCCN(CCO)CCCCOC(=O)C(CCCCCC)CCCCCCCC]

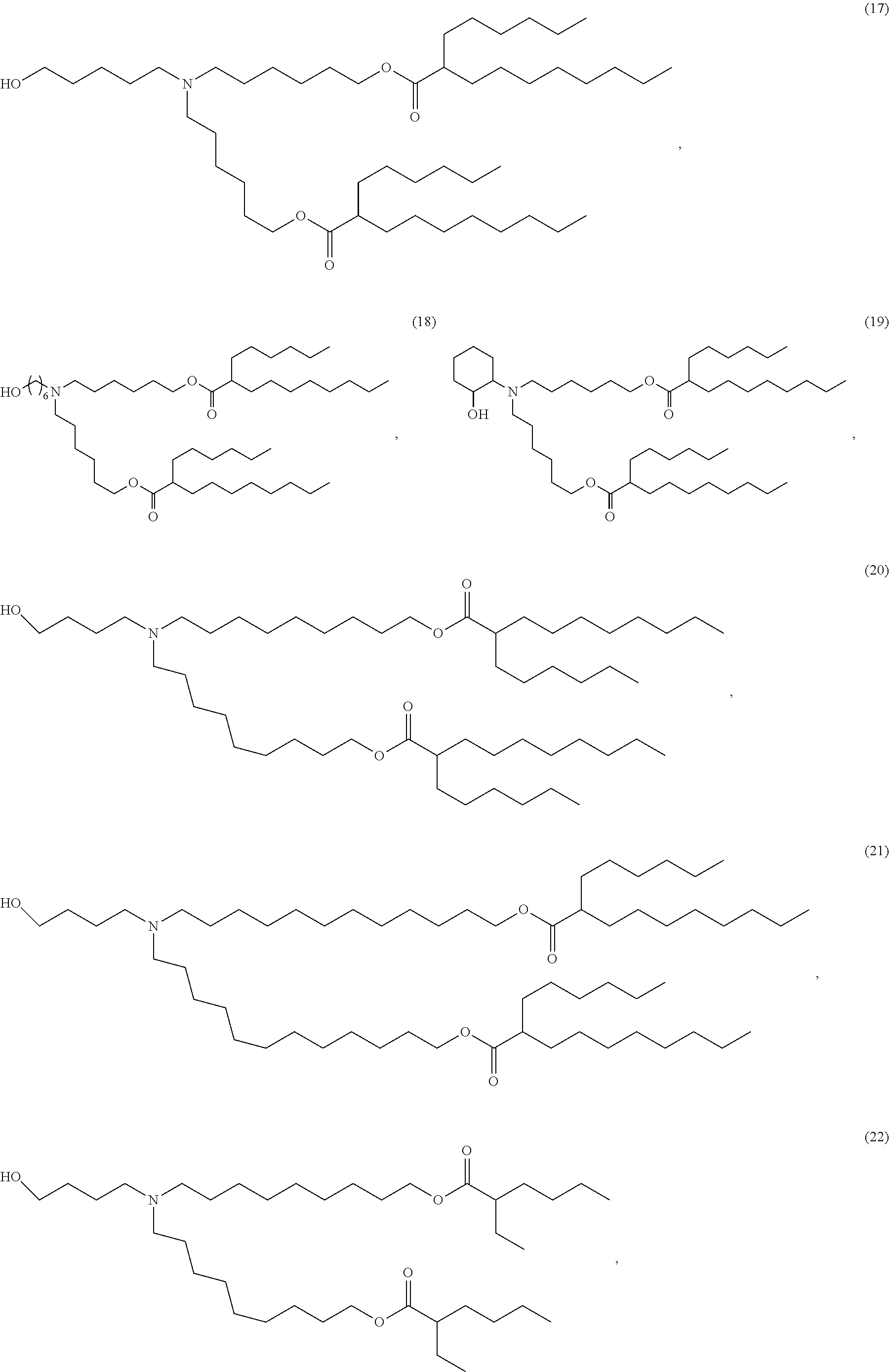

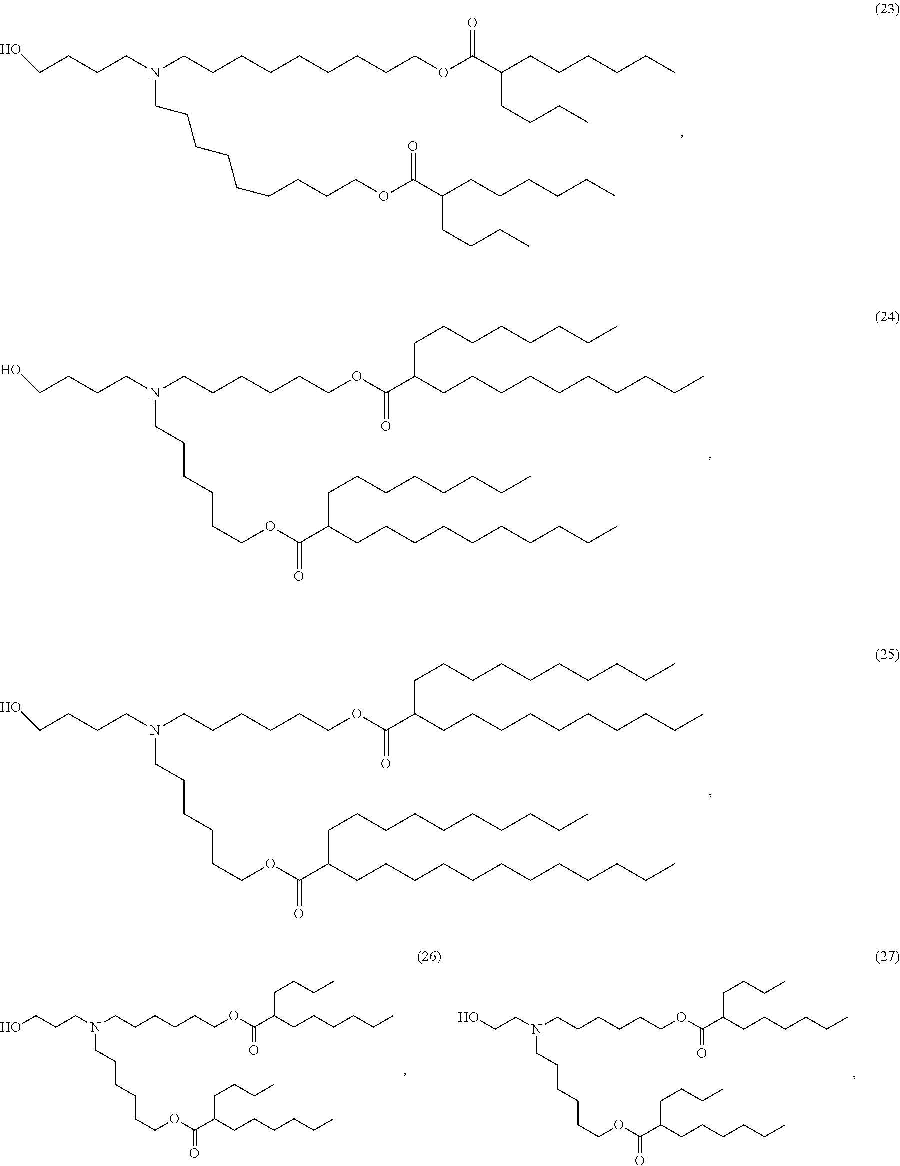 [SMILES: CCCCCCC(CCCC)C(=O)OCCCCCCCCCN(CCCCO)CCCCCCCCCOC(=O)C(CCCC)CCCCCC ; CCCCCCCCCCC(CCCCCCCC)C(=O)OCCCCCCN(CCCCO)CCCCCCOC(=O)C(CCCCCCCC)CCCCCCCCCC ; CCCCCCCCCCCCC(CCCCCCCCCC)C(=O)OCCCCCCN(CCCCO)CCCCCCOC(=O)C(CCCCCCCCCC)CCCCCCCCCC ; CCCCCCC(CCCC)C(=O)OCCCCCCN(CCCO)CCCCCCOC(=O)C(CCCC)CCCCCC ; CCCCCCC(CCCC)C(=O)OCCCCCCN(CCO)CCCCCCOC(=O)C(CCCC)CCCCCC]

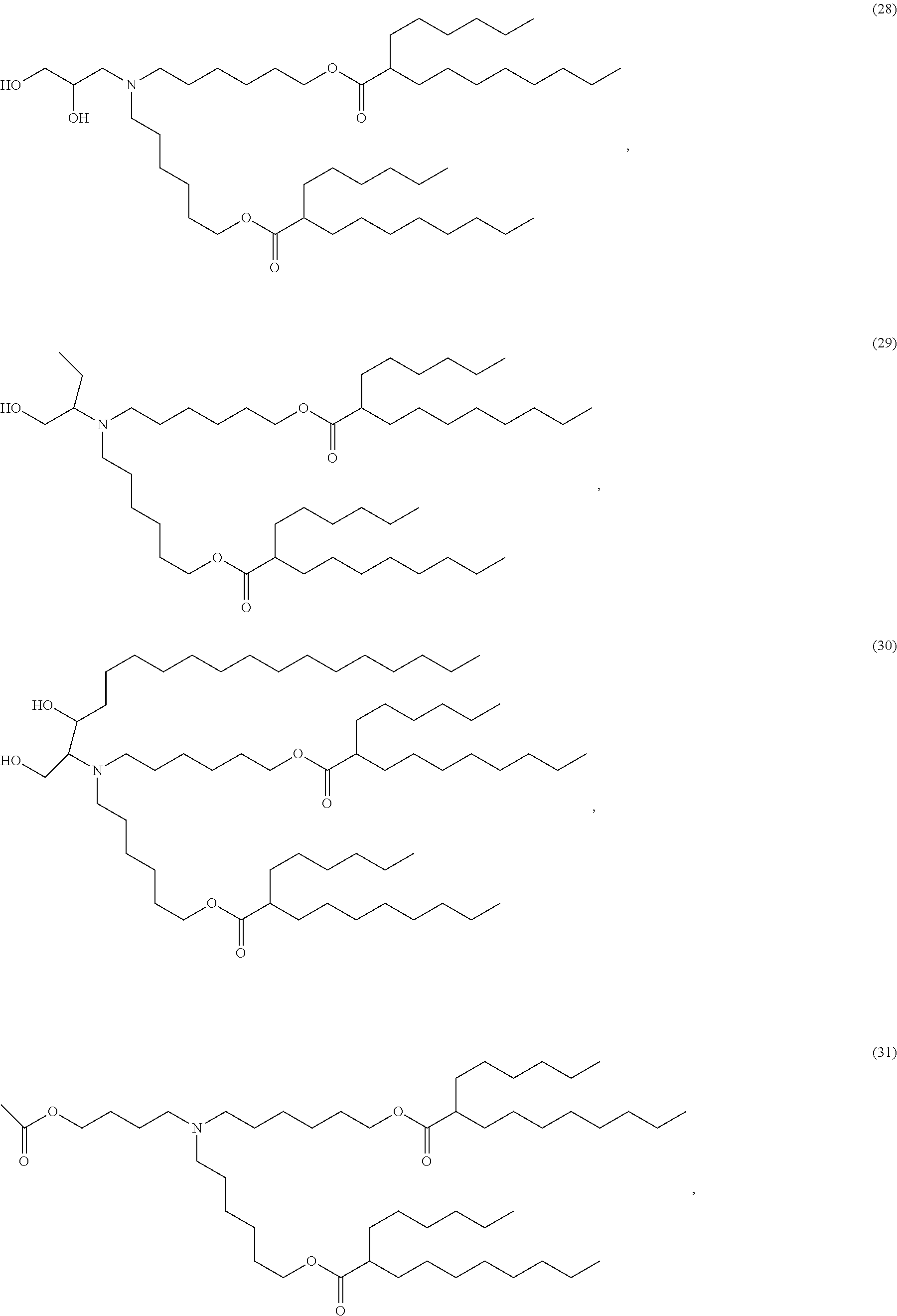

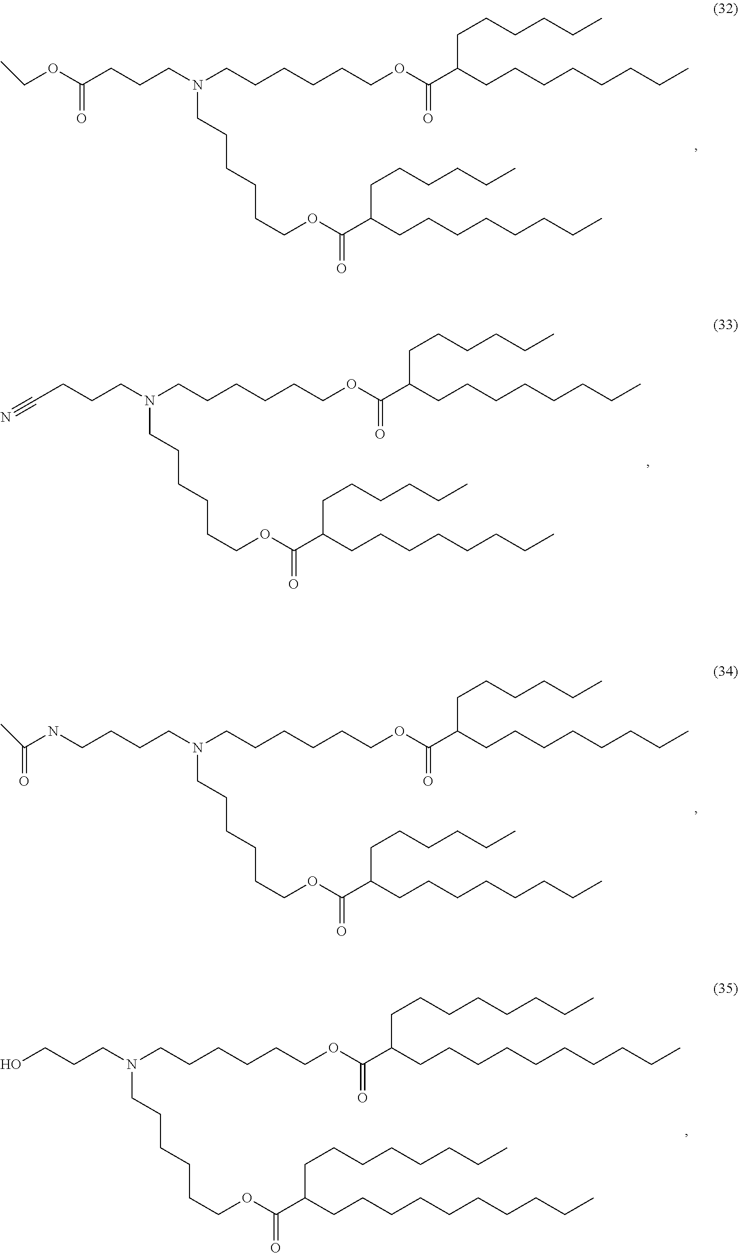

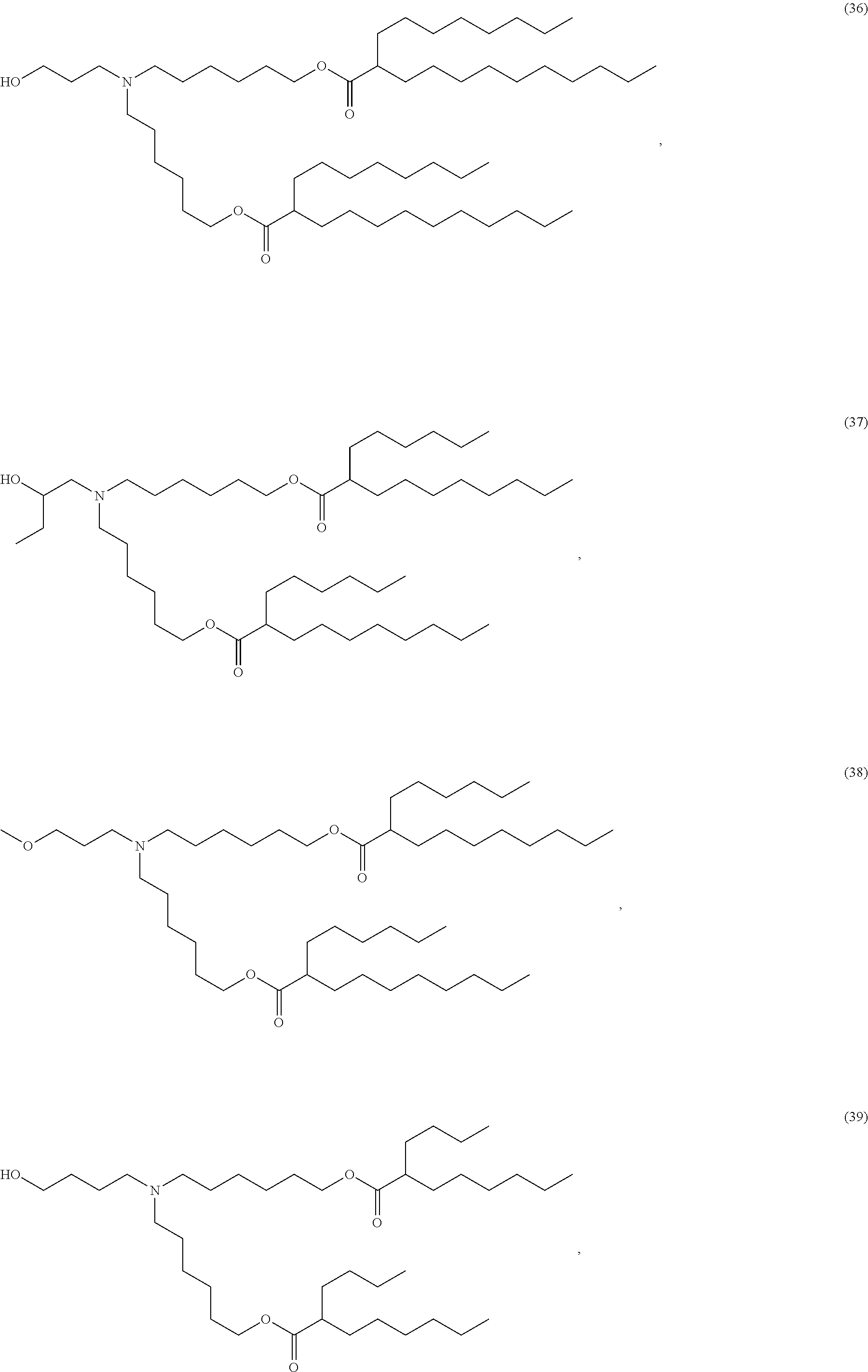

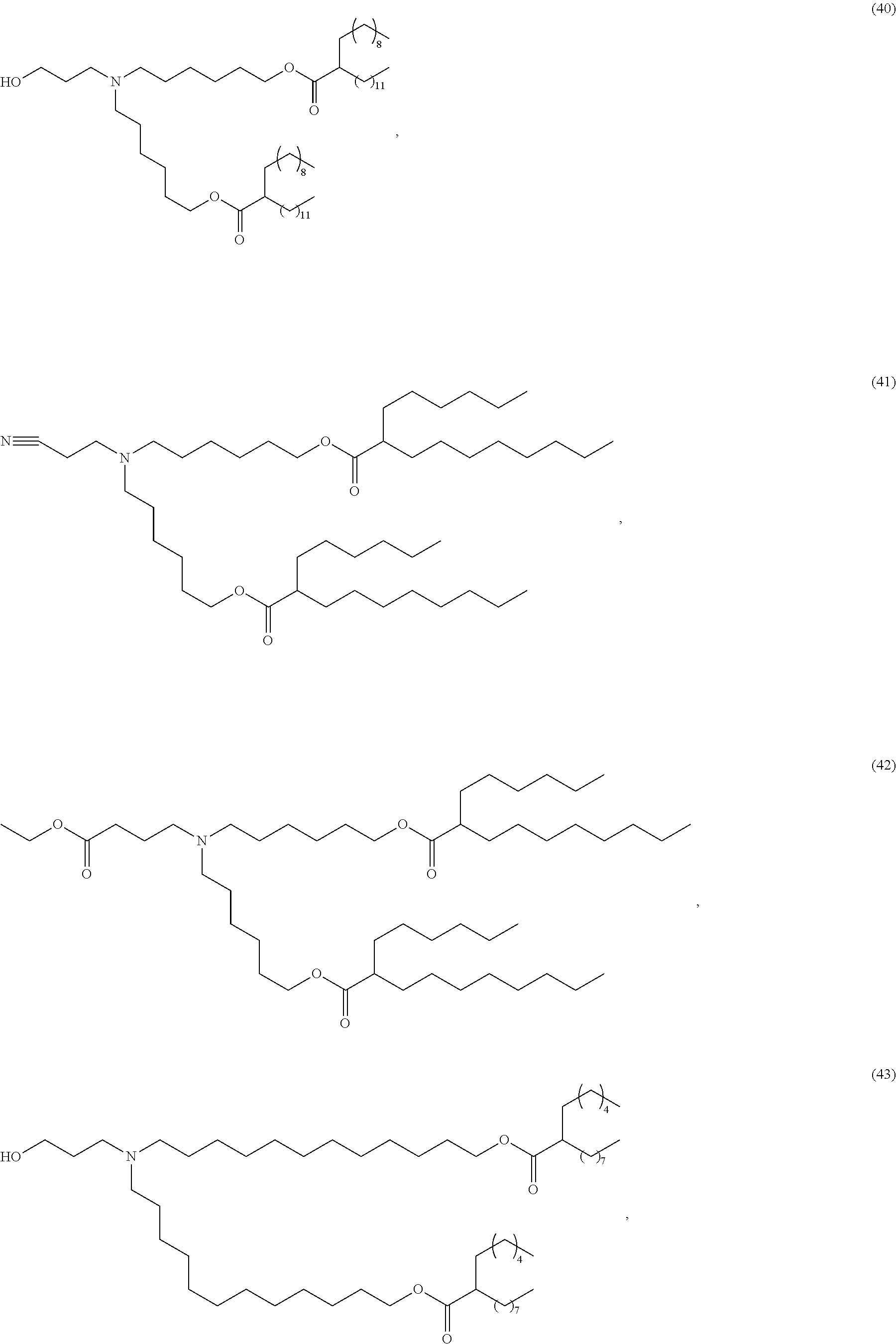

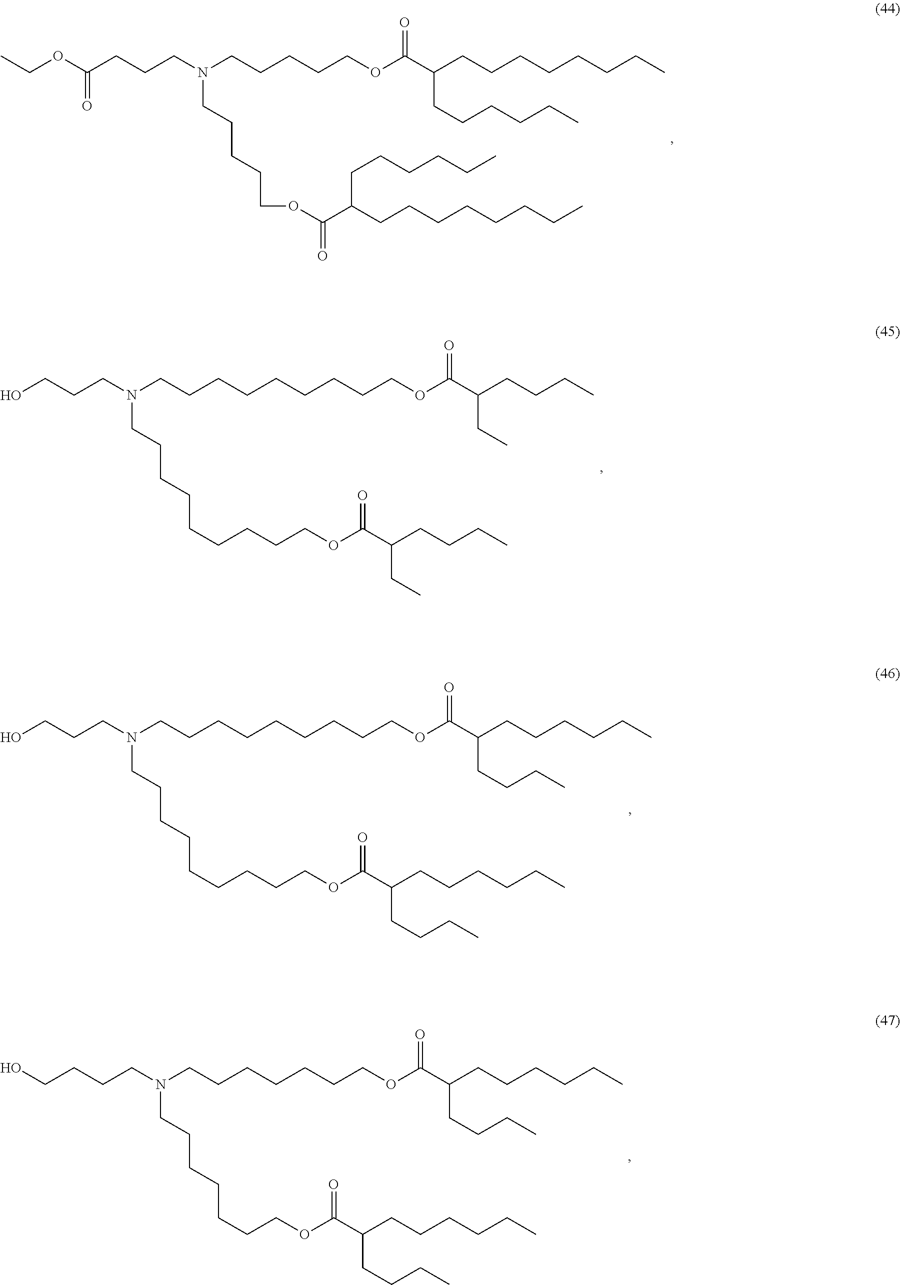 [SMILES: CCCCCCCCC(CCCCCC)C(=O)OCCCCCN(CCCCCOC(=O)C(CCCCCC)CCCCCCCC)CCCC(=O)OCC ; CCCCC(CC)C(=O)OCCCCCCCCCN(CCCO)CCCCCCCCCOC(=O)C(CC)CCCC ; CCCCCCC(CCCC)C(=O)OCCCCCCCCCN(CCCO)CCCCCCCCCOC(=O)C(CCCC)CCCCCC ; CCCCCCC(CCCC)C(=O)OCCCCCCCN(CCCCO)CCCCCCCOC(=O)C(CCCC)CCCCCC]

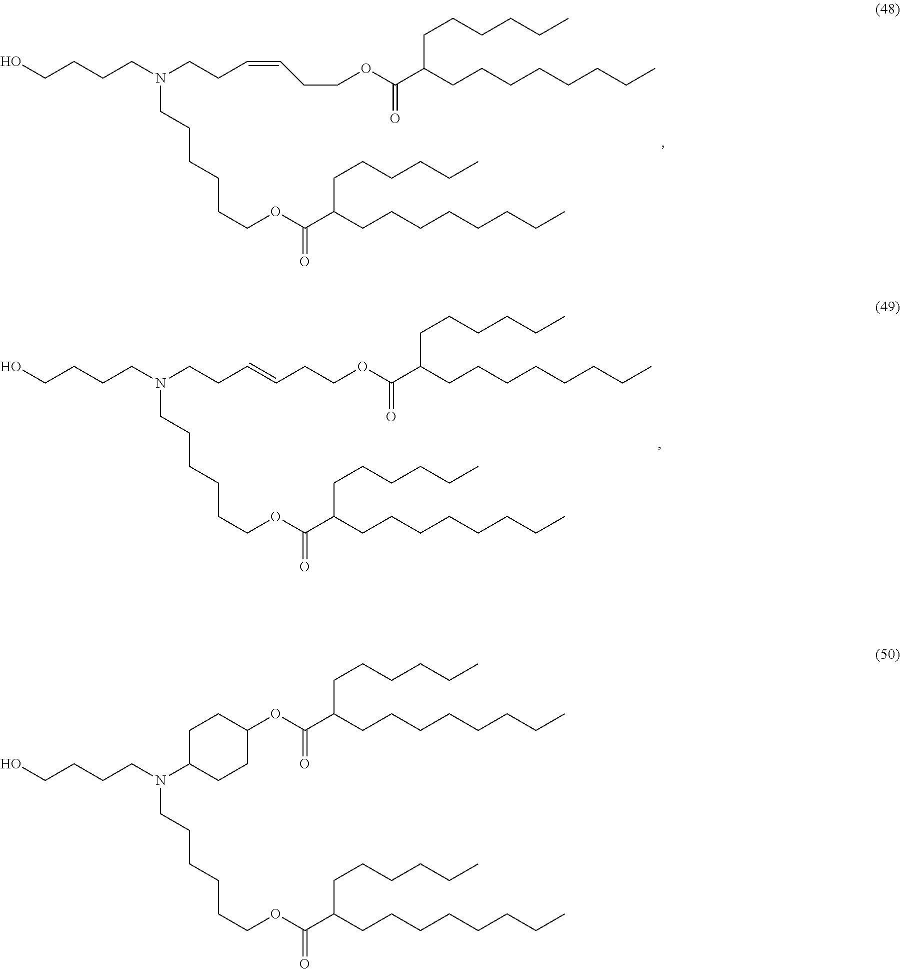 [SMILES: CCCCCCCCC(CCCCCC)C(=O)OCC/C=C\CCN(CCCCO)CCCCCCOC(=O)C(CCCCCC)CCCCCCCC ; CCCCCCCCC(CCCCCC)C(=O)OCC/C=C/CCN(CCCCO)CCCCCCOC(=O)C(CCCCCC)CCCCCCCC ; CCCCCCCCC(CCCCCC)C(=O)OCCCCCCN(CCCCO)C1CCC(OC(=O)C(CCCCCC)CCCCCCCC)CC1]

In certain aspects, the compounds of Formula I has the structure of Formula (13). In certain aspects, one or more compounds of Formula I described herein is excluded from the compounds of Formula I.

II. Methods of preparing compounds of Formula I and salts thereof and intermediates thereof

The compounds of Formula I can be prepared by i) reacting a first fatty acid having a chemical formula of R¹—COOH with a first oxychloride to form a first acyl chloride having a chemical formula of R¹—(CO)—Cl, and reacting a second fatty acid having a chemical formula of R²—COOH with a second oxychloride to form a second acyl chloride having a chemical formula of R²—(CO)—Cl; ii) reacting the first acyl chloride with a first diol having a chemical formula of HO-L′—CH₂—OH to form a first ester alcohol having a chemical formula of R¹—C(O)—O-L¹—CH₂—OH, and reacting the second acyl chloride with a second diol having a chemical formula of HO—L²—CH₂—OH to form a second ester alcohol having a chemical formula of R²—C(O)—O-L²—CH₂—OH; iii) oxidizing the first ester alcohol with a first oxidizing agent to form a first ester aldehyde having a chemical formula of R¹—C(O)—O-L′—CHO, and oxidizing the second ester alcohol with a second oxidizing agent to form a second ester aldehyde having a chemical formula of R²-C(O)-O-L²—CHO; and iv) reducing the first and second ester aldehyde in presence of a reducing agent and an amine having a chemical formula of R³-L³—NH₂, to form the compound of Formula I. R¹, R², R³, L¹, L², and L³ can be as defined above.

The first acyl chloride and the second acyl chloride can be formed in the same reaction medium or separately. The first ester alcohol and the second ester alcohol can be formed in the same reaction medium or separately. The first ester aldehyde and the second ester aldehyde can be formed in the same reaction medium or separately. In certain aspects, i) R¹ and R² are the same; ii) L¹ and L² are the same; iii) the first fatty acid and the second fatty acid are the same; iv) the first oxychloride and the second oxychloride are the same; v) first diol and the second diol are the same; vi) the first oxidizing agent and second oxidizing agent are the same; vii) the first acyl chloride and the second acyl chloride are the same and are formed in the same reaction medium; viii) the first ester alcohol and the second ester alcohol are the same and are formed in the same reaction medium; and ix) the first ester aldehyde and the second ester aldehyde are the same and are formed in the same reaction medium. In certain aspects, i) R¹ and R² are different; ii) L¹ and L² are the same or different; iii) the first acyl chloride and the second acyl chloride are formed separately; iv) the first ester aldehyde and the second ester aldehyde are formed separately, and v) the first ester aldehyde and the second ester aldehyde are formed separately. In certain aspects, i) R¹ and R² are the same; ii) L¹ and L² are different; iii) the first acyl chloride and the second acyl chloride are formed in the same reaction medium or separately; iv) the first ester aldehyde and the second ester aldehyde are formed separately, and v) the first ester aldehyde and the second ester aldehyde are formed separately. In certain aspects, i) R¹ and R² are different, and/or ii) L¹ and L² are different and the method optionally includes or excludes separating the compound of Formula I, from other lipids formed by reduction of the first ester aldehyde and the second ester aldehyde with the amine.

Certain aspects are directed to a cationic lipid (e.g., of Formula I or Formula 50) described herein, an intermediate for the production thereof (e.g., the acyl chloride, ester alcohol, ester aldehyde, and/or ester ketone), a pharmaceutically acceptable salt of the lipid, and/or pharmaceutically acceptable salt of the intermediate. Certain aspects are directed to a composition containing a cationic lipid described herein, an intermediate for the production thereof (e.g., the acyl chloride, ester alcohol, ester aldehyde, and/or ester ketone), a pharmaceutically acceptable salt of the lipid, and/or pharmaceutically acceptable salt of the intermediate, wherein the lipid and the intermediate is synthesized with a method described herein. In certain aspects, the composition contains a lipid having the structure of Formula (13), or a pharmaceutically acceptable salt thereof. Certain aspects, are directed of a use of a cationic lipid described herein, an intermediate for the production thereof (e.g., the acyl chloride, ester alcohol, ester aldehyde, and or ester ketone), a pharmaceutically acceptable salt of the lipid, and/or pharmaceutically acceptable salt of the intermediate.

### A. Formation of the Acyl Chloride.

The acyl chloride can be formed according to Scheme I. The oxychloride can be thionyl chloride, phosphoryl chloride, oxalyl chloride, or any combinations thereof. In certain aspects, the oxychloride is oxalyl chloride. In certain aspects, a stoichiometric excess of the oxychloride is used, and the reaction conditions of the fatty acid (e.g., first and/or the second fatty acid) and the oxychloride include contacting the fatty acid and the oxychloride at a molar ratio of, equal to any one of, at least any one of, at most any one of, or between any two of 1:1, 1: 1.01, 1: 1.02, 1: 1.03, 1: 1.04, 1: 1.05, 1: 1.06, 1:1.07, 1:1.08, 1:09, 1:1.1, 1:1.2, 1:1.3, 1:1.4, or 1:1.5 (or any range derivable therein). Stoichiometric excess of the oxychloride can increase yield of the acyl chloride. In certain aspects, a solution containing the fatty acid is contacted with a solution containing the oxychloride. In some aspects, the oxychloride solution further contains one or more organic solvents. In certain aspects, the oxychloride solution contains dichloromethane (DCM). In some aspects, the fatty acid solution further contains one or more organic solvents. In certain aspects, the fatty acid solution contains DCM. In some instances, the oxychloride is added to the reaction at a rate to control the rate of off-gassing, such as to avoid a high rate of off gassing that is unsafe.

In some aspects, reaction conditions of the fatty acid and the oxychloride include a reaction temperature of, equal to any one of, at least any one of, at most any one of, or between any two of 15, 16, 17, 18, 19, 20, 21, 22, 23, 24, 25, 26, 27, 28, 29, or 30° C. (or any range derivable therein).

In some aspects, the fatty acid and the oxychloride reaction is catalyzed with a catalyst. In some particular aspect, the catalyst is dimethylformamide (DMF). In certain aspects, equal to any one of, at least any one of, at most any one of, or between any two of 0.001, 0.002, 0.003, 0.004, 0.005, 0.006, 0.007, 0.008, 0.009, or 0.01 moles (or any range derivable therein) of DMF, per mole of the fatty acid is contacted with the fatty acid and oxychloride. In certain aspects, the yield of the acyl chloride is, equal to any one of, at least any one of, or between any two of 95, 96, 97, 98, 99, or 99.5% or any range derivable therein. In certain aspects, one or more step(s) and/or reagent(s) described herein (e.g., for formation of the acyl chloride) are excluded.

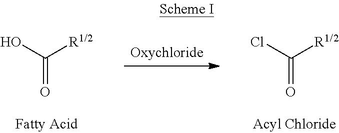

B. Formation of the Ester Alcohol from Acyl Chloride

The ester alcohol can be formed from the acyl chloride and a diol according to Scheme II. In certain aspects, the method excludes i) isolation and/or purification of the acyl chloride, such as by column chromatography, from the reaction medium in which the acyl chloride is formed (e.g., reaction medium of the fatty acid and oxychloride), and/or ii) reaction of an isolated and/or purified (e.g., by column chromatography) acyl chloride with the diol. The acyl chloride and the diol can be reacted in presence of a tertiary amine. In certain aspects, the tertiary amine is triethylamine. In certain aspects, a stoichiometric excess of the diol is used in the reaction of the acyl chloride and the diol. In certain aspects, the reaction conditions of the acyl chloride and the diol include contacting the acyl chloride and the diol at a molar ratio of, equal to any one of, at least any one of, at most any one of, or between any two of 0.8:3.5, 0.8:3.4, 0.8:3.3, 0.9:3.2, 0.9:3.1, 1:3, 1:2.9, 1:2.8, 1.1:2.7, 1:1, 1:2.6, or 1:2.5 (or any ranges or values in between). In certain aspects, the reaction conditions of the acyl chloride and the diol include a temperature of, equal to any one of, at least any one of, at most any one of, or between any two of 15, 16, 17, 18, 19, 20, 21, 22, 23, 24, 25, 26, 27, 28, 29, or 30° C. (or any range derivable therein).

In some aspects, the method further includes adding a base to a esterification-product mixture formed by the reaction of the acyl chloride and diol. The esterification-product mixture can contain i) the ester alcohol, ii) optionally unreacted reactants such as oxychloride and/or diol, and iii) optionally side products and/or byproducts formed in the reaction of the fatty acid and oxychloride, and/or the acyl chloride and diol. In some aspects, one or more of i), ii), or iii) is excluded. The base can remove at least a portion of the unreacted reactants, side products, and/or byproducts from the esterification-product mixture, such as oxalate impurities generated from excess oxychloride (e.g., oxalyl chloride) and the diol (e.g., 1,6-hexanediol) . In certain aspects, an alkaline aqueous solution containing the base is added to the esterification-product mixture, to form a biphasic medium. The biphasic medium can contain an organic phase containing the ester alcohol and an aqueous phase. In certain aspects, the base is sodium hydroxide. In certain aspects, the alkaline aqueous solution has a pH 10 or greater, such as equal to any one of, at least any one of, at most any one of, or between any two of 10, 11, 12, 13, or 14 (or any range derivable therein). In certain aspects, the biphasic medium is heated to reflux. For example, refluxed at a temperature, equal to any one of, at least any one of, at most any one of, or between any two of 30, 31, 32, 33, 34, 35, 36, 37, 38, 39, 40, 41, 42, 43, 44, 45, 46, 47, 48, 49, and 50° C. (or any range derivable therein).

In certain aspects, after the reflux the organic phase (e.g., containing the ester alcohol), and the aqueous phase are separated, and the organic phase is washed with a first wash solution having a pH 4 or below, such equal to any one of, at least any one of, at most any one of, or between any two of 4, 3, 2, 1, 0.01 (or any range derivable therein), and a second wash solution having a pH, equal to any one of, at least any one of, at most any one of, or between any two of 5, 5.5, 6, 6.5, 7, 7.5, 8, 8.5, or 9 (or any range derivable therein). In certain aspects, the first wash solution contains hydrogen chloride, such as aqueous solution of hydrogen chloride. The organic phase is washed with the first wash solution and the second wash solution in any suitable order.

In certain aspects, the acyl chloride conversion, for the reaction of the acyl chloride and diol, is greater than 97%, such as, equal to any one of, at least any one of, or between any two of 97, 98, 99, 99.5, 99.6, 99.7, 99.8, 99.9, 99.95, 99.99, or 100% or any range derivable therein. The ester alcohol yield, from the reaction of the acyl chloride and diol can be equal to any one of, at least any one of, or between any two of 75, 76, 77, 78, 79, 80, 81, 82, 83, 84, 85, 86, 87, 88, 89, or 90% (or any range derivable therein). In certain aspects, one or more step(s) and/or reagent(s) described herein (e.g., for formation of the ester alcohol from acyl chloride) are excluded.

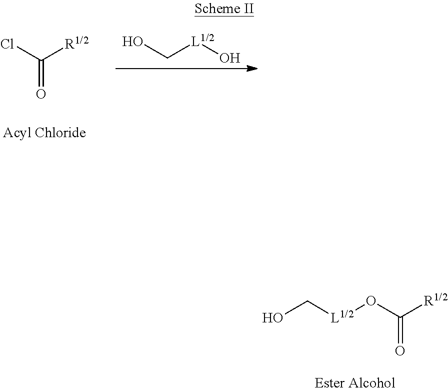

C. Formation of the Ester Ketone from Acyl Chloride

The ester ketone can be formed from the acyl chloride and a ketone alcohol according to Scheme III, wherein z is an integer ranging from 0 to 10 and the ketone alcohol optionally includes a carbocyclic ring of from 5 to 10 carbon atoms, wherein the carbon atom (z=0) or alkyl group (z=1-10) bearing the alcohol hydroxyl group can be attached to any non-ketone-bearing carbon atom in the carbocyclic ring. In some aspects, the ketone alcohol is 4-hydroxycyclohexan-1-one. In certain aspects, the method excludes i) isolation and/or purification of the acyl chloride, such as by column chromatography, from the reaction medium in which the acyl chloride is formed (e.g., reaction medium of the fatty acid and oxychloride), and/or ii) reaction of an isolated and/or purified (e.g., by column chromatography) acyl chloride with the ester alcohol. The acyl chloride and the ketone alcohol can be reacted in presence of a tertiary amine. In certain aspects, the tertiary amine is triethylamine. In certain aspects, a stoichiometric excess of the ketone alcohol is used in the reaction of the acyl chloride and the ketone alcohol. In certain aspects, the reaction conditions of the acyl chloride and the ketone alcohol include contacting the acyl chloride and the ketone alcohol at a molar ratio of, equal to any one of, at least any one of, at most any one of, or between any two of 0.8:3.5, 0.8:3.4, 0.8:3.3, 0.9:3.2, 0.9:3.1, 1:3, 1:2.9, 1:2.8, 1.1:2.7, 1:1, 1:2.6, or 1:2.5 (or any ranges or values in between). In certain aspects, the reaction conditions of the acyl chloride and the ketone alcohol include a temperature of, equal to any one of, at least any one of, at most any one of, or between any two of 15, 16, 17, 18, 19, 20, 21, 22, 23, 24, 25, 26, 27, 28, 29, or 30° C. (or any range derivable therein).

In some aspects, the method further includes adding a base to an esterification-product mixture formed by the reaction of the acyl chloride and ketone alcohol. The esterification-product mixture can contain i) the ester ketone, ii) optionally unreacted reactants such as oxychloride and/or ketone alcohol, and iii) optionally side products and/or byproducts formed in the reaction of the fatty acid and oxychloride, and/or the acyl chloride and ketone alcohol. In some aspects, one or more of i), ii), or iii) is excluded. The base can remove at least a portion of the unreacted reactants, side products, and/or byproducts from the esterification-product mixture, such as oxalate impurities generated from excess oxychloride (e.g., oxalyl chloride) and the ketone alcohol (e.g., 4-hydroxycyclohexan-1-one). In certain aspects, an alkaline aqueous solution containing the base is added to the esterification-product mixture, to form a biphasic medium. The biphasic medium can contain an organic phase containing the ester ketone and an aqueous phase. In certain aspects, the base is sodium hydroxide. In certain aspects, the alkaline aqueous solution has a pH 10 or greater, such as equal to any one of, at least any one of, at most any one of, or between any two of 10, 11, 12, 13, or 14 (or any range derivable therein). In certain aspects, the biphasic medium is heated to reflux. For example, refluxed at a temperature, equal to any one of, at least any one of, at most any one of, or between any two of 30, 31, 32, 33, 34, 35, 36, 37, 38, 39, 40, 41, 42, 43, 44, 45, 46, 47, 48, 49, and 50° C. (or any range derivable therein).

In certain aspects, after the reflux, the organic phase (e.g., containing the ester ketone) and the aqueous phase are separated, and the organic phase is washed with a first wash solution having a pH 4 or below, such equal to any one of, at least any one of, at most any one of, or between any two of 4, 3, 2, 1, 0.01 (or any range derivable therein), and a second wash solution having a pH, equal to any one of, at least any one of, at most any one of, or between any two of 5, 5.5, 6, 6.5, 7, 7.5, 8, 8.5, or 9 (or any range derivable therein). In certain aspects, the first wash solution contains hydrogen chloride, such as aqueous solution of hydrogen chloride. The organic phase is washed with the first wash solution and the second wash solution in any suitable order.

In certain aspects, the acyl chloride conversion, for the reaction of the acyl chloride and ketone alcohol, is greater than 97%, such as, equal to any one of, at least any one of, or between any two of 97, 98, 99, 99.5, 99.6, 99.7, 99.8, 99.9, 99.95, 99.99, or 100% or any range derivable therein. The ester ketone yield, from the reaction of the acyl chloride and ketone alcohol can be equal to any one of, at least any one of, or between any two of 75, 76, 77, 78, 79, 80, 81, 82, 83, 84, 85, 86, 87, 88, 89, or 90% (or any range derivable therein). In certain aspects, one or more step(s) and/or reagent(s) described herein (e.g., for formation of the ester ketone from acyl chloride) are excluded.

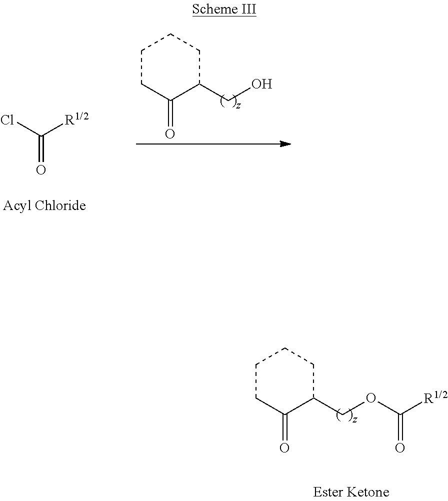

D. Formation of the Ester Aldehyde from the Ester Alcohol

The ester aldehyde can be formed from the ester alcohol according to Scheme IV. The ester alcohol, (e.g., synthesized as described above) can be oxidized with an oxidizing agent to form the ester aldehyde. In certain aspects, the method excludes i) isolation and/or purification of the ester alcohol, such as by column chromatography, from the washed organic phase, e.g., the organic phase obtained after washing with the first and second wash solution, and/or ii) oxidation of an isolated purified (such as by column chromatography) ester alcohol. In certain aspects, the ester alcohol in the washed organic phase is contacted with the oxidizing agent to form the ester aldehyde.

The oxidizing agent can contain sodium hypochlorite. In certain aspects, the sodium hypochlorite is sodium bicarbonate treated sodium hypochlorite. The sodium bicarbonate treated sodium hypochlorite can be formed by contacting sodium bicarbonate with sodium hypochlorite at a molar ratio of 0.2:1 to 0.5:1. The ester alcohol and the sodium hypochlorite, such as sodium bicarbonate treated sodium hypochlorite, can be contacted at a molar ratio of, equal to any one of, at least any one of, at most any one of, or between any two of 1:1, 1:1.01, 1:1.02, 1:1.03, 1:1.04, 1:1.05, 1:1.06, 1:1.07, 1:1.08, 1:1.09, 1:1.1, 1:1.2, 1:1.3, 1:1.4, or 1:1.5 (or any range derivable therein). In certain aspects, the oxidation of the ester alcohol is catalyzed with a oxidation catalyst. In some particular aspects, the oxidation catalyst is potassium bromide and/or 2,2,6,6-tetramethylpyridine N-oxide (TEMPO). In certain aspects, oxidation reaction conditions include contacting the ester alcohol with, equal to any one of, at least any one of, at most any one of, or between any two of 0.05, 0.06, 0.07, 0.08, 0.09, 0.1, 0.11, 0.12, 0.13, 0.14, or 0.15 (or any range derivable therein) moles of potassium bromide per mole of ester alcohol and/or, equal to any one of, at least any one of, at most any one of, or between any two of 0.005, 0.006, 0.007, 0.008, 0.009, 0.01, 0.011, 0.012, 0.013, 0.014, or 0.015 (or any range derivable therein) moles of TEMPO per mole of ester alcohol.

The ester alcohol is oxidized at a temperature equal to or below 15° C., such as, equal to any one of, at most any one of, or between any two of 15, 14, 13, 12, 11, 10, 9, 8, 7, 6, 5, 4, 3, 2, 1, 0, -1, -2, -3, -4, and -5° C. (or any range derivable therein). Oxidation of the ester alcohol at a temperature equal to or below 15° C. can reduced and/or prevent over oxidation of the ester alcohol. In certain aspects, the ester alcohol (e.g., the washed organic medium containing the ester alcohol) and the oxidizing agent is contacted at a rate sufficient to keep the temperature of reaction medium formed by contacting, at equal to or below 15° C.

In certain aspects, the oxidation-product mixture formed by the oxidation of the ester alcohol with the oxidizing agent is washed with a first oxidation-wash solution and a second oxidation-wash solution. The oxidation-product mixture can contain the ester aldehyde formed by oxidation. The first oxidation-wash solution can have a pH 4 or below, such 4, 3, 2, 1, 0.01 (or any range derivable therein). In certain aspects, the first oxidation-wash solution contains hydrogen chloride. The second oxidation-wash solution can contain, equal to any one of, at least any one of, at most any one of, or between any two of 5, 6, 7, 8, 9, 10, 11, 12, 13, 14, and 15 wt. % of sodium thiosulfate. Washing with the first and second oxidation-wash solution can be performed at any suitable order. The ester aldehyde yield, from the oxidation of the ester alcohol can be, equal to any one of, at least any one of, or between any two of 90, 91, 92, 93, 94, 95, 96, 97, 98, 99, or 99.5% (or any range derivable therein). The washing with the first and second oxidation-wash solution can remove at least a portion of the unreacted reactants, such as oxychloride, catalyst (e.g., DMF), tertiary amine, oxidizing agent, and/or oxidation catalyst from the oxidation-product mixture. In certain aspects, one or more step(s) and/or reagent(s) described herein (e.g., for formation of the ester aldehyde from the ester alcohol) are excluded.

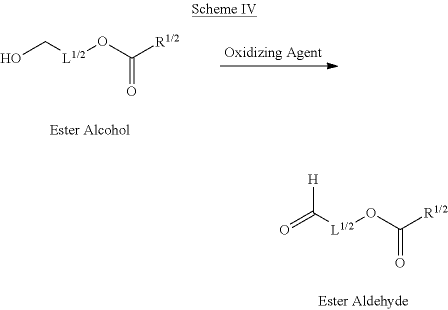

E. Formation of the Compound of Formula I from the Ester Aldehyde

A compound of Formula I can be formed from a first ester aldehyde and a second ester aldehyde according to Scheme V. The first ester aldehyde and the second ester aldehyde can be the same or different, and can be formed as described above. In certain aspects, the method excludes i) isolation and/or purification of the ester aldehyde(s) (e.g., first and second ester aldehyde), such as by column chromatography, from the washed oxidation-product mixture, (e.g., obtained after washing the oxidation-product mixture with the first and second oxidation-wash solution), and/or ii) reduction of isolated purified (e.g., by column chromatography) ester aldehyde(s). In certain aspects, ester aldehyde(s) in the washed oxidation-product mixture(s) is contacted with an amine and a reducing agent to reduce the ester aldehyde(s) and form the compound of Formula I. In some aspects, reaction between ester aldehyde(s) and an amine is a reductive amination reaction. In some aspects, the aldehyde carbon of the first ester aldehyde and the aldehyde carbon of the second ester aldehyde are part of or become part of L¹ and L², respectively In some aspects, the ester aldehyde is contacted with the amine at an ester aldehyde (total, e.g., first and second) and amine molar ratio of, equal to any one of, at least any one of, at most any one of, or between any two of 1:1, 1.5:1, 1.9:1, 2:1, 2.1: 1, 2.2:1, 2.3:1, 2.4: 1, 2.5:1, 2.6:1, 2.7:1, 2.8:1, 2.9:1, or 3:1 (or any range derivable therein). In some particular aspects, the ester aldehyde is reacted with the amine at a ester aldehyde (total) and amine molar ratio of, equal to any one of, at least any one of, at most any one of, or between any two of 2:1, 2.1:1, 2.2:1, 2.3:1, 2.4:1, or 2.5:1 (or any range derivable therein).

In some aspects, the first ester aldehyde and the second ester aldehyde are different, the molar ratio of the first and second ester aldehydes in the reduction reaction is equal to any one of, at least any one of, at most any one of, or between any two of 1:2, 3:4, 4:5, 0.9:1, 1:1, 1:0.9, 5:4, 4:3, or 2:1. In some particular aspects, the molar ratio of the first and second ester aldehydes in the reduction reaction is 0.9:1, 1:1, or 1:0.9 (or any range derivable therein).

In some aspects, the reducing agent contains a hydride. In some particular aspects, the hydride is sodium triacetoxyborohydride. In certain aspects, the ester aldehyde (total) is contacted with sodium triacetoxyborohydride at a molar ratio of, equal to any one of, at least any one of, at most any one of, or between any two of 2:3, 2:3.5, 2:3.9, 2:4, 2:4.1, 2:4.2, 2:4.3, 2:4.4, 2:4.5, 2:4.6, 2:4.7, 2:4.8, 2:4.9, or 2:5 (or any range derivable therein). In some aspects, the ester aldehyde(s) is reduced with the hydride at a temperature of 30° C. or lower, such as, equal to any one of, at most any one of, or between any two of 30, 29, 28, 27, 26, 25, 24, 23, 22, 21, 20, 19, 18, 17, 16, 15, 14, 13, 12, 11, or 10° C. (or any range derivable therein).

In some aspects, the reduction of the ester aldehyde(s) with the amine and the hydride is quenched with a base. In certain aspects, equal to any one of, at least any one of, at most any one of, or between any two of 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, or 5 moles (or any range derivable therein) of the base per mole of ester aldehyde (total) reduced, is used for quenching. In certain aspects, the base is sodium hydroxide. In certain aspects, an alkaline aqueous solution containing the base is added to a reaction medium of the reduction reaction to quench the reduction reaction, and form a biphasic product mixture containing an aqueous phase, and an organic phase containing the compound of Formula I. In some instances, the use of the base removes the need to use acetic acid and desiccant (mol. sieves) to drive the reaction to completion. In some instances, the method excludes use of an acid and/or a desiccant at this step.

The conversation of the ester aldehyde (each) can be, equal to any one of, at least any one of, or between any two of 85, 86, 87, 88, 89, 90, 91, 92, 93, 94, 95, 96, 97, 98, or 99% (or any range derivable therein). In some aspects, the yield of compound of Formula I from the reduction of ester aldehyde(s) is, equal to any one of, at least any one of, or between any two of 80, 81, 82, 83, 84, 85, 86, 87, 88, 89, 90, 91, 92, 93, 94, 95, 96, 97, or 98%.

In some aspects, the method further includes or excludes adding a an organic solvent to the biphasic product mixture. In some particular aspects, the method includes adding DCM to the biphasic product mixture.

In certain aspects, when the hydride, such as sodium triacetoxyborohydride, is used as the reducing agent, the method excludes or sufficiently excludes (e.g., added in amounts less than 0.05, less than 0.01, or less than 0.005, or less than 0.001 molar equivalent of the ester aldehyde reduced) addition of acetic acid and/or desiccant (e.g., molecular sieves) to the reduction reaction medium.

In certain aspects, the reducing agent contains hydrogen (H₂). The reduction of the ester aldehyde(s) with the amine and hydrogen can be catalyzed with a metal catalyst. In some aspects, the metal catalyst contains a platinum (Pt), palladium (Pd), ruthenium (Ru), rhodium (Rh), and/or iridium (Ir) catalyst. In some particular aspects, the metal catalyst contains platinum (Pt) on carbon. In some aspects, the ester aldehyde(s) is reduced with hydrogen at a temperature of, equal to any one of, at least any one of, at most any one of, or between any two of 25, 26, 27, 28, 29 30, 31, 32, 33, 34, 35, 36, 37, 38, 39, 40, 41, 42, 43, 44, or 45° C. (or any range derivable therein).

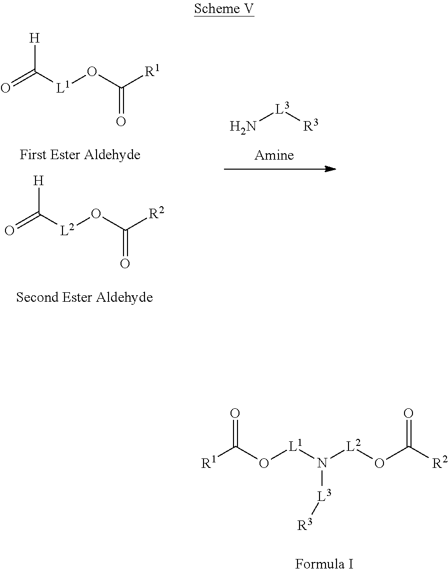

In certain aspects, the method further includes purifying the compound of Formula I. In some aspects, the compound of Formula I is purified by extraction. The extraction solvent can be using an organic solvent, an inorganic solvent, or a combination thereof. In some aspects, the solvent is n-heptane, methanol, or an aqueous solution, or a combination thereof. In some aspects, the solvent is a 10% aqueous methanol solution. In some aspects, the compound of Formula I is comprised in n-heptane and is extracted with a 10% aqueous methanol solution to remove polar impurities. In some aspects, the compound of Formula I is subsequently or alternatively purified by silica gel chromatography or polymer resin chromatography. In some aspects, the extraction mother liquor is used as a feed for the chromatography step. In some aspects, the extraction mother liquor is concentrated prior to being provided as a feed for the chromatography step. In certain aspects, compound of Formula I in the product solution (e.g., formed through quenching of the reductive amination reaction) is purified by silica gel chromatography or polymer resin chromatography to form the purified compound of Formula I.

In certain aspects, the method further includes or excludes, purifying the compound of Formula I via distillation. In certain aspects, the compound of Formula I in the organic phase of the biphasic product mixture is distilled. In some aspects, the extraction mother liquor from extraction-based purification is distilled. In certain aspects, a solution obtained from eluting the silica gel chromatography column or polymer resin chromatography is distilled. In some aspects, purifying the compound of Formula I includes purification by extraction, silica gel or polymer resin chromatography, and/or distillation. In some aspects, the distillation process includes, contacting the compound of Formula I, with n-heptane to form a n-heptane solution, distilling the n-heptane solution at i) a temperature, equal to any one of, at least any one of, at most any one of, or between any two of 30, 31, 32, 33, 34, 35, 36, 37, 38, 39, 40, 41, 42, 43, 44, or 45° C. (or any range derivable therein) and/or ii) a pressure, equal to any one of, at least any one of, at most any one of, or between any two of 0, 0.05, 0.1, 0.15, 0.2, 0.25, or 0.3 bar (or any range derivable therein) to form a first distillation residue, contacting the first distillation residue with ethanol to form an ethanol solution and, distilling the ethanol solution at a) a temperature, equal to any one of, at least any one of, at most any one of, or between any two of 30, 31, 32, 33, 34, 35, 36, 37, 38, 39, 40, 41, 42, 43, 44, or 45° C. (or any range derivable therein), and/or ß) a pressure, equal to any one of, at least any one of, at most any one of, or between any two of 0, 0.05, 0.1, 0.15, 0.2, 0.25, or 0.3 bar (or any range derivable therein) to form a second distillation residue comprising compound of Formula I. In certain aspects, the compound of Formula I in the organic phase of the biphasic product mixture is contacted with n-heptane to form the n-heptane solution. The second distillation residue can contain i) less than 5000, or less than 4000, or less than 3000, or less than 2000, or less than 1000 parts per million by weight (ppmw) of n-heptane and less than 50000, or less than 40000, or less than 30000, or less than 20000, or less than 10000, or less than 5000 ppmw of ethanol. In certain aspects, the second distillation residue contains, equal to any one of, at least any one of, or between any two of 95, 96, 97, 98, 99, or 99.5 wt. % of the compound(s) of Formula I. In certain aspects, one or more step(s) and/or reagent(s) described herein (e.g., for formation of the compound of Formula I from ester aldehyde) are excluded.

In certain aspects, the cationic lipid has a chemical formula of Formula (48), (49), or (50), or a salt thereof. The cationic lipids of Formula (48), (49), or (50) can be synthesized using a method similar to the synthesis method of compound I described herein, where the first and/or second diol in Scheme I can be cis- 3-hexene-1,6-diol (for Formula (48)), trans-3-hexene-1,6-diol (for Formula (49)), or 1, 4 cyclohexanediol (for Formula (50)) respectively. In certain aspects, the method includes, a) reacting a first fatty acid with oxalyl chloride to form a first acyl chloride, and reacting a second fatty acid with oxalyl chloride to form a second acyl chloride (e.g., according to the conditions described in Scheme I); b) reacting the first acyl chloride with a first diol to form a first ester alcohol, and reacting the second acyl chloride with a second diol to form a second ester alcohol (e.g., according to the conditions described in Scheme II); c) oxidizing the first ester alcohol to form a first ester aldehyde, and oxidizing the second ester alcohol to form a second ester aldehyde (e.g., according to the conditions described in Scheme III); and d) reducing the first and second ester aldehyde in presence of sodium triacetoxyborohydride and 4-amino-1-butanol to form the compound of Formula (48), (49), or (50) (e.g., according to the conditions described in Scheme IV), wherein the first and second fatty acid has the formula of

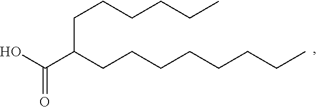 [SMILES: CCCCCCCCC(CCCCCC)C(=O)O]

the first diol is cis-3-hexene-1,6-diol (for Formula 48), trans-3-hexene-1,6-diol (for Formula 49), or 1, 4 cyclohexanediol (for Formula 50), and the second diol is 1,6 hexane-diol.

### III. Salts of the Cationic Lipids and Intermediates Thereof

The salts of the cationic lipids can have the chemical formula of Formula III:

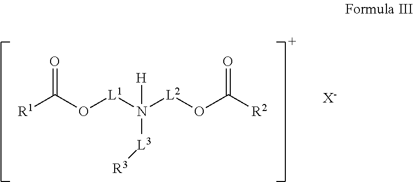

wherein R¹, R², R³, L¹, L², and L³ can be as defined above. X⁻ can be an anion. In certain aspects, X⁻ can be chloride, bromide, iodide, sulfate, acetate, mesylate, tosylate, (1R)-(—)-10-camphorsulfonate, 1,2-ethanedisulfonate, oxalate, dibenzoyl-L-tartarate, phosphate, L-tartarate, maleate, fumarate, succinate, or malonate.

In some particular aspects, the salt has the structure of Formula V

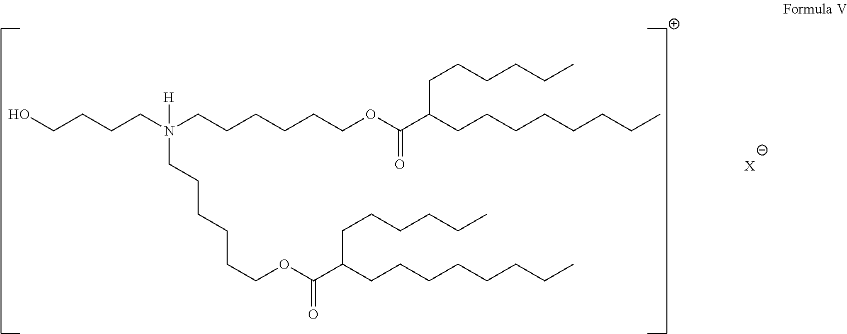

The salt can be formed by contacting a compound of Formula I with an acid having a chemical formula of HX. In certain aspects, the acid is hydrochloric acid, hydrobromic acid, hydroiodic acid, sulfuric acid, acetic acid, methanesulfonic acid, toluenesulfonic acid, (1R)-(—)-10-camphorsulfonic acid, 1,2-ethanedisulfonic acid, oxalic acid, dibenzoyl-L-tartaric acid, phosphoric acid, L-tartaric acid, maleate, fumaric acid, succinic acid, or malonic acid. In certain aspects, the salt of the cationic lipid is in a crystallized form. In certain aspects, one or more salts (e.g., of Formula III) described herein are excluded.

Certain aspects are directed to salts of intermediates produced in the production of the cationic lipid. In some aspects, the salts have the chemical formula of Formula IV:

([R¹—C(O)—O-L¹—CH₂—O]⁻)ₓM^{x+} Formula IV

wherein R¹ and L¹ can be as defined above. M^{x+} can be a cation, and x can be an integer. In certain aspects, x is 1 or 2, and M^{x+} is Na⁺, K⁺, Ca²⁺ and Mg²⁺. In certain particular aspects, R¹ have the structure of formula (6), and/or L¹ is —(CH₂)₅—. The salt (e.g., of Formula IV) can be formed by contacting a compound of R¹—C(O)—O-L¹—CH₂—OH with an base having a chemical formula of M(OH)ₓ. In some particular aspects the base is NaOH, KOH, Ca(OH)₂, and/or Mg(OH)₂. In certain aspects, the salt (e.g., of Formula IV) is in a crystallized form. In certain aspects, one or more salts (e.g., of Formula IV) described herein are excluded.

### IV. Use of Compounds of Formula I, Intermediates Thereof, and Salts Thereof; and Compositions Containing the Compound of Formula I, Intermediates Thereof, and Salts Thereof

Certain aspects are directed of a use of a cationic lipid described herein, an intermediate for the production thereof (e.g., the acyl chloride, ester alcohol, and/or ester aldehyde), a pharmaceutically acceptable salt of the lipid, and/or pharmaceutically acceptable salt of the intermediate. Certain aspects are directed to a composition containing a cationic lipid described herein, an intermediate for the production thereof (e.g., the acyl chloride, ester alcohol, and/or ester aldehyde), a pharmaceutically acceptable salt of the lipid, and/or pharmaceutically acceptable salt of the intermediate. The cationic lipid described herein, and the intermediate can be synthesized using a method described herein.

The cationic lipids and/or pharmaceutically acceptable salts thereof, optionally in combination with other lipids, can be used for intracellular delivery of a therapeutic agent. In certain aspects, the therapeutic agent can be a nucleic acid. In certain aspects, the nucleic acid can be messenger RNA (mRNA), nucleoside-modified mRNA, antisense oligonucleotides, ribozymes, DNAzymes, plasmids, immune stimulating nucleic acids, antagomirs, anti-miRs, miRNA mimics, supermirs, and/or aptamers. In some particular aspects, the nucleic acid can be antisense, plasmid DNA, and/or nucleoside-modified mRNA.

Certain aspects, are directed to a pharmaceutical composition containing a cationic lipid described herein, an intermediate for the production thereof (e.g., the acyl chloride, ester alcohol, and/or ester aldehyde), a pharmaceutically acceptable salt of the lipid, and/or pharmaceutically acceptable salt of the intermediate; and a therapeutic agent. In certain aspects, the cationic lipid, the intermediate and/or the pharmaceutically acceptable salt thereof can be in a lipid nanoparticle form. The lipid nanoparticle can have at least one dimension on the order of nanometers (e.g., 1-1,000 nm), and can include one or more lipids. In some aspects, the lipid nanoparticle can further include or exclude one or more excipient selected from neutral lipids, charged lipids, steroids, and polymer conjugated lipids. In some aspects, the therapeutic agent, such as the nucleoside-modified RNA, is encapsulated in the lipid portion of the lipid nanoparticle or an aqueous space enveloped by some or all of the lipid portion of the lipid nanoparticle, thereby protecting it from enzymatic degradation or other undesirable effects induced by the mechanisms of the host organism or cells, e.g., an adverse immune response. In certain aspects, the lipid nanoparticles have an average diameter of from about, equal to any one of, at least any one of, at most any one of, or between any two of 30 nm to about 150 nm, about 40 nm to about 150 nm, about 50 nm to about 150 nm, about 60 nm to about 130 nm, about 70 nm to about 110 nm, about 70 nm to about 100 nm, about 80 nm to about 100 nm, about 90 nm to about 100 nm, about 70 to about 90 nm, about 80 nm to about 90 nm, about 70 nm to about 80 nm, or equal to any one of, at least any one of, at most any one of, or between any two of about 30 nm, 35 nm, 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, or 150 nm, and are substantially non-toxic. In certain embodiments, the nucleoside-modified RNA, when present in the lipid nanoparticles, is resistant in aqueous solution to degradation by a nuclease.

Administration of the compositions described herein can be carried out via any of the accepted modes of administration of agents for serving similar utilities. Pharmaceutical compositions may be formulated into preparations in solid, semi-solid, liquid or gaseous forms, such as tablets, capsules, powders, granules, ointments, solutions, suspensions, suppositories, injections, inhalants, gels, microspheres, and aerosols. Typical routes of administering such pharmaceutical compositions include, without limitation, oral, topical, transdermal, inhalation, parenteral, sublingual, buccal, rectal, vaginal, and intranasal. The term parenteral as used herein includes subcutaneous injections, intravenous, intramuscular, intradermal, intrasternal injection, or infusion techniques. Pharmaceutical compositions described herein are formulated so as to allow the active ingredients contained therein to be bioavailable upon administration of the composition to a patient. Compositions that will be administered to a subject or patient take the form of one or more dosage units, where for example, a tablet may be a single dosage unit, and a container of a compound in aerosol form may hold a plurality of dosage units. The composition to be administered will, in any event, contain a therapeutically effective amount of a compound within the scope of this disclosure, or a pharmaceutically acceptable salt thereof, for treatment of a disease or condition of interest in accordance with the teachings described herein.

A pharmaceutical composition within the scope of this disclosure may be in the form of a solid or liquid. In one aspect, the carrier(s) are particulate, so that the compositions are, for example, in tablet or powder form. The carrier(s) may be liquid, with the compositions being, for example, an oral syrup, injectable liquid, or an aerosol, which is useful in, for example, inhalator administration. When intended for oral administration, the pharmaceutical composition is preferably in either solid or liquid form, where semi-solid, semi-liquid, suspension, and gel forms are included within the forms considered herein as either solid or liquid. As a solid composition for oral administration, the pharmaceutical composition may be formulated into a powder, granule, compressed tablet, pill, capsule, chewing gum, wafer or the like form. Such a solid composition will typically contain one or more inert diluents or edible carriers. In addition, one or more of the following may be present or exclude: binders such as carboxymethylcellulose, ethyl cellulose, microcrystalline cellulose, gum tragacanth, or gelatin; excipients such as starch, lactose, or dextrins; disintegrating agents such as alginic acid, sodium alginate, Primogel, corn starch and the like; lubricants such as magnesium stearate or Sterotex; glidants such as colloidal silicon dioxide; sweetening agents such as sucrose or saccharin; a flavoring agent such as peppermint, methyl salicylate, or orange flavoring; and a coloring agent. When the pharmaceutical composition is in the form of a capsule, for example, a gelatin capsule, it may contain, in addition to materials of the above type, a liquid carrier such as polyethylene glycol or oil. The pharmaceutical composition may be in the form of a liquid, for example, an elixir, syrup, solution, emulsion or suspension. The liquid may be for oral administration or for delivery by injection, as two examples. When intended for oral administration, preferred composition contain, in addition to the present compounds, one or more of a sweetening agent, preservatives, dye/colorant, and flavor enhancer. In a composition intended to be administered by injection, one or more of a surfactant, preservative, wetting agent, dispersing agent, suspending agent, buffer, stabilizer, and isotonic agent may be included or exclude.

A liquid pharmaceutical composition, whether they be solutions, suspensions or other like form, may include or exclude one or more of the following adjuvants: sterile diluents such as water for injection, saline solution, preferably physiological saline, Ringer's solution, isotonic sodium chloride, fixed oils such as synthetic mono or diglycerides which may serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates, or phosphates; and agents for the adjustment of tonicity such as sodium chloride or dextrose; agents to act as cryoprotectants such as sucrose or trehalose. The parenteral preparation can be enclosed in ampoules, disposable syringes, or multiple dose vials made of glass or plastic. Physiological saline is a preferred adjuvant. An injectable pharmaceutical composition is preferably sterile.

A liquid pharmaceutical composition intended for either parenteral or oral administration should contain an amount of a compound such that a suitable dosage will be obtained.

The pharmaceutical compositions may be prepared by methodology well known in the pharmaceutical art. For example, a pharmaceutical composition intended to be administered by injection can be prepared by combining the lipid nanoparticles with sterile, distilled water or other carrier so as to form a solution. A surfactant may be added to facilitate the formation of a homogeneous solution or suspension. Surfactants are compounds that non-covalently interact with a compound consistent with the teachings herein so as to facilitate dissolution or homogeneous suspension of the compound in the aqueous delivery system.

The compositions within the scope of the disclosure, or their pharmaceutically acceptable salts, are administered in a therapeutically effective amount, which will vary depending upon a variety of factors including the activity of the specific therapeutic agent employed; the metabolic stability and length of action of the therapeutic agent; the age, body weight, general health, gender, and diet of the patient; the mode and time of administration; the rate of excretion; the drug combination; the severity of the particular disorder or condition; and the subject undergoing therapy.

### EXAMPLES

The invention is further described in detail by reference to the following experimental examples. These examples are provided for purposes of illustration only and are not intended to be limiting unless otherwise specified. Thus, the invention should in no way be construed as being limited to the following examples, but rather, should be construed to encompass any and all variations which become evident as a result of the teaching provided herein.

### Example 1

### Producing an Ester Alcohol from 2-Hexyldeconoic Acid and 1,6-Hexanediol

An ester alcohol (C-2) was formed according to the Scheme E1.

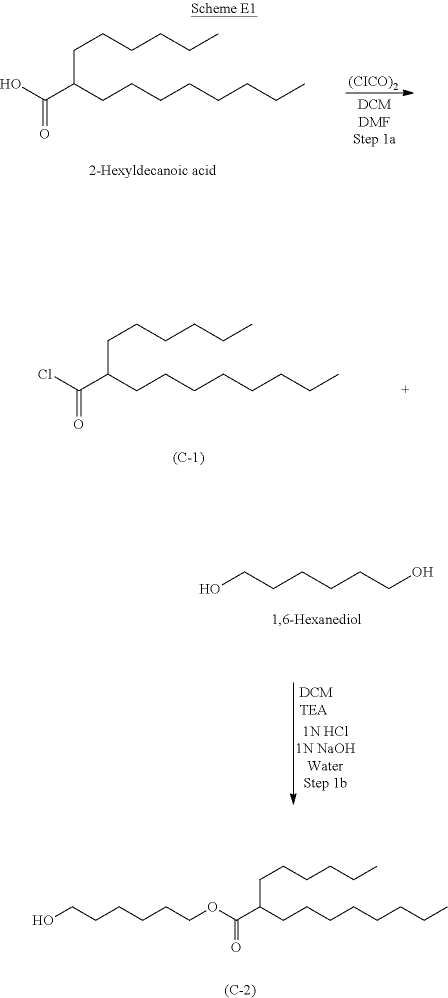

Material used for forming the ester alcohol (C-2) and amount of intermediate acyl chloride (C-1) and (C-2) produced is listed in Table 1. Equiv. and eq are used for equivalent.

TABLE 1
Material used

| | CAS | MW or | | Weight or | | | Molar Ratio | | |
|---|---|---|---|---|---|---|---|---|---|
| Materials | No | Density | | Volume | | mmoles | or ml/g | | Comments |
| Oxalyl Chloride | 79-37-8 | 126.93 | | 3.41 | mL | 39.31 | 1.05 | equiv. | Reactant |
| | | | | 4.99 | g | | | | |
| Dichloromethane | 75-09-2 | 1.463 | g/mL | 20 | mL | | 2 | mL/g | Solvent |
| 2-Hexyldecanoic | 25354-97-6 | 256.4 | | 10 | g | | 1 | | Limiting |
| acid | | | | | | | | | Reagent |
| Dichloromethane | 75-09-2 | 1.463 | g/mL | 50 | mL | | 5 | mL/g | Solvent |
| (Acid) | | | | | | | | | |
| DMF | 68-12-2 | 73.09 | | 0.015 | mL | | 0.005 | eq | Catalyst |
| (C-1) | | 274.9 | | 10.29 | g | | 1.0 | eq | Non-isolated |
| | | | | | | | | | intermediate |
| 1,6-Hexanediol | 629-11-8 | 118.17 | | 13.4 | g | 112.3 | 3.0 | equiv. | Reagent |
| Dichloromethane | 75-09-2 | 1.463 | g/mL | 50 | mL | | 5 | mL/g. | Solvent |
| (Diol) | | | | | | | | | |
| Triethylamine | 121-44-8 | 101.19 | | 6.52 | mL | 46.79 | 1.25 | | Reagent |
| | | | | 4.74 | g | | | | |
| Hydrochloric acid | 7647-01-0 | 36.46 | | 80 | mL | 82.32 | 8 | mL/g | Reagent |
| (1N) | | | | | | | | | |
| Sodium Hydroxide | 1310-73-2 | 40.0 | | 112 | mL | 112.3 | 3.0 | eq | Reagent |
| (1N) | | 1.04 | g/mL | | | | | | |

| | | | | | | | |
|---|---|---|---|---|---|---|---|
| Water | 7732-18-5 | 18.015 | 100 | mL | 10 | mL/g | Reagent |
| (C-2) | | 356.6 | 13.35 | g | | | Product |

The ester alcohol (C-2) was synthesized according to the steps listed in Table 2.

TABLE 2
Method steps for synthesizing ester alcohol (C-2)

| Unit | | |
|---|---|---|
| Op | Description of the steps | Notes/Observations |
| 1. | To a reactor charge Dichloromethane | Start agitation, ambient temperature, ensure scrubber |
| | (20.0 mL, 2.0 mL/g-LR) | is functioning. All charges are based on 2- |
| | | Hexyldecanoic acid. The reaction has also been run at |
| | | a total of 6 mL/g-LR |
| 2. | To reactor of Unit Op (“UO”) 1. | |
| | (“UO 1”) charge Oxalyl chloride | |
| | (3.41 mL, 4.99 g, 1.05 equivalent) | |
| 3. | In a separate vessel charge | |
| | Dichloromethane (50.0 mL, 5.0 | |
| | mL/g-LR) | |
| 4. | In the same vessel as UO 3, charge 2- | The acid is an oil and mobile |
| | hexyldecanoic acid (10.0 g, LR) | |
| 5. | In the same vessel as UO 3, charge | This is a catalyst |
| | DMF (0.015 mL, 0.005 equivalents) | |
| 6. | Charge mixture from UO 2 to UO 5 | The reaction is endothermic with gas evolution being |
| | over 15 min | dose controlled. The solution from UO 2 has been |
| | | charged at various rates (0.065 to 5.0 mL/min) with |
| | | no impact to quality. |
| 7. | Maintain 20-25° C. for 2 h. | The reaction is typically completed within an hour of |
| | | complete dosing from UO 6. Some off-gassing will |
| | | continue until the reaction is complete (~1 hr) |
| 8. | Upon reaction completion (as | This reaction has been run up to 24 h at 25° C. with |
| | determined by Gas Chromatography | minor impact to yield and quality (loss of ~3% |
| | (GC)), charge solution from UO 7 to | potency). Reaction completion is not more than |
| | solution in UO 11 - See UO 12. | (NMT) 2% 2-hexyldecanoic acid as determined by |
| | | Gas Chromatography. If reaction completion is not |
| | | achieved, charge additional 0.05 eq oxalyl chloride to |
| | | reach reaction completion |
| 9. | To a separate reactor charge | Start agitation, ambient temperature, ensure scrubber |
| | Dichloromethane (50.0 mL, 5.0 | is functioning. All charges are based on 2- |
| | mL/g-LR) | Hexyldecanoic acid. The second half of this reaction |
| | | has also been run at a total of 10 mL/g-LR |
| 10. | In the same vessel as UO 9, charge | Solids may require 30 min to dissolve as the |
| | 1,6-hexanediol (13.4 g, 3.0 | dissolution is endothermic. Proceed to next step |
| | equivalents) | despite solids not being dissolved |
| 11. | In the same vessel as UO 9, charge | No exotherm noted - hold with stirring until all solids |
| | Triethylamine (6.52 mL, 4.74 g, 1.25 | dissolved before progressing to UO 12. Solution may |
| | equivalents) | be heated to dissolve solids and returned to room |
| | | temp prior to charging of (C-1). |
| 12. | Charge solution from UO 7 ((C-1) | Charge at rate such that reaction temperature (Tr) = |
| | mixture) into mixture from UO 11. | 20 ± 5° C. Initial addition is mildly exothermic with |
| | | light floculent mist appearing (TEA•HCl) |
| 13. | Hold 25° C. for at least 2 h and check | No solids noticed, reaction is typically complete |
| | for reaction completion | within an hour - This is an acceptable hold point. |
| | | Gas Chromatography and Thin Layer |
| | | Chromatography used for determination of residual |
| | | (C-1). |
| 14. | Charge Sodium Hydroxide (1N) (112 | This addition is slightly exothermic. |
| | mL, 3.0 equiv) | |
| 15. | Heat reaction mixture to 40° C. and | |
| | hold for 180 min | |
| 16. | Sample for Ion-Pair Chromatography | Check IPC (1H NMR) of organic layer for |
| | (IPC) | disappearance of peaks at 1.7 and 4.2 ppm. |
| 17. | Cool to 25° C. | |
| 18. | Stop agitation | Bottom layer is Organic (desired) and may be slightly |
| | | hazy, top layer is aqueous (undesired) with pH 14 |
| 19. | Collect bottom organic layer | Top layer (basic) can be combined with upcoming |
| | | acidic wash (UO 24) |
| 20. | Charge UO 19 bottom (organic layer) | |
| | to vessel | |
| 21. | Charge Hydrochloric acid (1N) (80 | This addition is slightly exothermic. Scrubber may be |
| | mL, 8 mL/g-LR) | turned off at this point |
| 22. | Hold at 25° C. for 15 min. | Agitation on |
| 23. | Stop agitation | Phase split is rapid and clean. Bottom layer is |
| | | Organic (desired), top layer is Aqueous (undesired) |
| 24 | Collect bottom organic layer | Top layer (Acidic) can be discarded after pH |
| | | adjustment with caustic |
| 25. | Charge UO 24 bottom (organic layer) | Start agitation |
| | to vessel and start agitation | |
| 26. | Charge water (100 mL, 10 mL/g LR) | No exotherm noted |
| 27. | Hold at 25° C. for 15 min. | Agitation on |
| 28. | Stop agitation | Phase split is rapid and clean. Bottom layer is |
| | | Organic (desired), top layer is Aqueous (not desired) |
| 29. | Collect bottom organic layer | Top layer is aqueous and can be combined with |
| | | previous aqueous wash (UO 17 and UO 24) |
| 30. | Charge UO 28 bottom layer (organic) | |
| | to vessel | |
| 31. | Concentrate to ~40 mL volume (~3 | Collect ~80 mL of DCM. Distillation may be |
| | mL/g LR) | conducted at Room Temperature under vacuum |
| 32. | Obtain crude (C-2) | Solution is ~25% wt/wt solution |

The yield of ester alcohol (C-2), from the above method was 78 to 82%.

### Example 2

### Producing an Ester Aldehyde (C-3) from Oxidation of the Ester Alcohol (C-2)

The ester alcohol (C-2) formed in Example 1, was oxidized to form an ester aldehyde (C-3), according to the Scheme E2.

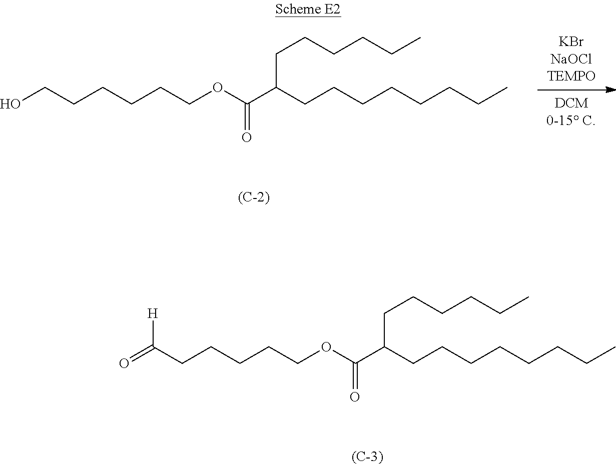 [SMILES: CCCCCCCCC(CCCCCC)C(=O)OCCCCCCO ; "(C-2)" ; CCCCCCCCC(CCCCCC)C(=O)OCCCCCC=O ; "(C-3)"]

Material used for oxidation of the ester alcohol (C-2) and production amount of (C-2) is listed in Table 3. Equiv. and eq are used for equivalent.

TABLE 3
Material used.

| | CAS | MW or | Weight or | | | Molar Ratio | | |
|---|---|---|---|---|---|---|---|---|
| Materials | No | Density | Volume | | mmoles | or ml/g | | Comments |
| (C-2) | | 356.6 | 100 | g | 280 | 1.00 | equiv. | Limiting |
| | | | | | | | | Reagent |
| Dichloromethane | 75-09-2 | 1.463 g/mL | 400 | mL | | 4.00 | ml/g | Solvent |
| Potassium Bromide | 7758-02-3 | 119 | 14.02 | mL | 28.0 | 0.10 | eq | Co-catalyst |
| (2N) | | | | | | | | |
| TEMPO (2,2,6,6- | 2564-83-2 | 156.24 | 0.44 | g | 2.80 | 0.01 | eq | Catalyst |
| tetramethylpyridine N- | | | | | | | | |
| oxide) | | | | | | | | |
| Sodium Hypochlorite | 7681-52-9 | 74.44 | 315 | mL | 350 | 1.25 | eq | Reagent |
| (1.1M) | | | | | | | | |
| Sodium bicarbonate | 144-55-8 | 84.01 | 10.6 | g | 128 | 0.45 | eq | Reagent |
| Hydrochloric acid (1N) | 7647-01-0 | 36.46 | 140 | mL | 140 | 0.5 | eq | Reagent |
| Sodium thiosulfate | 7772-98-7 | 158.1 | 890 | mL | 563 | 2.0 | equiv. | Reagent |
| (10% wt/wt) | | | | | | | | |
| (C-3) | | 354.6 | 99.44 | g | | | | Product |

The ester alcohol (C-2) was oxidized according to the steps listed in Table 4, to synthesize the ester aldehyde (C-3).

TABLE 4
Method steps for oxidation of ester alcohol (C-2) and formation of ester aldehyde (C-3)

| Unit Op | Description of the steps | Notes/Observations |
|---|---|---|
| 1. | To reactor charge | All charges are based on (C-2). The reaction has also |
| | Dichloromethane (400.0 mL, 4.0 | been run at a total of 10 mL/g-LR |
| | mL/g-LR) | |
| 2. | Start Agitation | |
| 3. | To reactor charge (C-2) (100 g, | (C-2) is an oil. (C-2) can be brought into this step as |
| | 280 mmol, LR) | a crude solution from step 1 at the approximately |
| | | same concentration (1 g/5 mL). If the (C-2) is a |
| | | dichloromethane solution then the solvent charge in |
| | | Unit Op 1 can be omitted |
| 4. | To reactor charge Potassium | 2M solution will result in biphasic mixture. Water |
| | Bromide (14.02 mL, 0.10 | followed by 0.1 eq of solid KBr could also be added |
| | equivalent) | instead of a pre-made solution |
| 5. | To the vessel charge TEMPO | Order of addtion of step 3, 4, 5 is not relavent. |
| | (0.44 g, 0.001 equivalent) | TEMPO is a catalyst |
| 6. | Set Tr to 0 ± 5° C. | |
| 7. | In separate vessel charge sodium | The sodium hypochlorite and sodium |
| | hypochlorite (315 mL, 1.25 eq) | bicarbonate mixture was prepared shortly before |
| | which has been treated with | use. Reaction may be complete after 1.0 eq of |
| | Sodium bicarbonate (9.2 g, 0.45 eq) | bleach. IPC can be used to confirm. If excess |
| | | sodium hypochlorite is charged, over oxidation |
| | | may occur generating the carboxylic acid instead |
| | | of the desired aldehyde. |
| 8. | Charge materials from UO 7 into | Initial addition is quite exothermic but becomes |
| | UO 6 at such a rate as to maintain | less so over the course of the addition. Reactions |
| | temp of <10° C. | have been allowed to warm to 15° C. with no |
| | | impact to quality. |
| 9. | Hold Tr at 0 ± 5° C. for 30 post | Reaction mixtures have been held for 15 h post |
| | complete addition of UO 8. | sodium hypochlorite addition. |
| 10. | Remove sample for IPC analysis. | NMT 5% (C-2) remaining. If IPC fails, charge |
| | IPC is by GC | additional sodium hypochlorite equal to amount |
| | | of (C-2) remaining. Buffer the hypochlorite |
| | | solution with 0.4 eq of sodium bicarbonate with |
| | | respect to amount of additional sodium |
| | | hypochlorite added. Hold 30 min then IPC (GC) |
| | | with NMT 5% (C-2) remaining. |
| 11. | Charge Hydrochloric acid (1N) | Minimal exothern with small amount of CO₂ |
| | (140 mL, 0.5 equivalents) | noted |
| 12. | Hold for 5 min then turn off | The HCl will break any emulsion that may form. |
| | agitation | The phase splits are fast and clear. |
| 13. | Isolate bottom (desired) organic | Discard top Aqueous layer after proper pH |
| | layer | adjustment. |
| 14. | Charge UO 13 isolated bottom | |
| | layer back to vessel. | |
| 15. | Turn on agitation | |
| 16. | Charge Sodium thiosulfate (10% | No exotherm noted |
| | wt/wt, 890 mL, 2.0 equivalents) | |
| 17. | Hold for 5 min then turn off | The sodium thiosulfate is charged to neutralize any |
| | agitation | residual sodium hypochlorite. The phase splits are |
| | | fast and clear. |
| 18. | Isolate bottom (desired) organic | Discard top Aqueous layer after proper pH |
| | layer | adjustment. |
| 19. | Charge UO 18 bottom layer | |
| | (organic) to vessel | |
| 20. | Concentrate to ~300 mL volume | Collect 200 mL of DCM. Distillation can be |
| | | conducted at Room Temperature under vacuum. |
| 21. | Obtain crude (C-3) | Solution is ~35% wt/wt solution |

The yield of (C-3), from the above method was 95 to 98%.

### Example 3

### Synthesizing a Cationic Lipid from Reduction of the Ester Aldehyde (C-3) using Triacetoxyborohydride

A portion of the ester aldehyde (C-3) synthesized in Example 2, was reduced with sodium triacetoxyborohydride and 4-amino-1-butanol to form a cationic lipid (C-4), according to the Scheme E3.

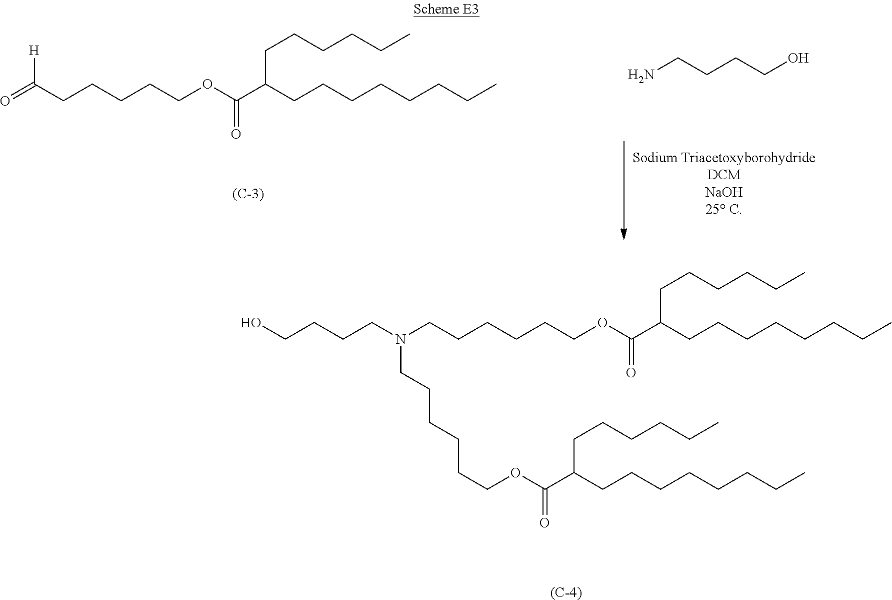 [SMILES: CCCCCCCCC(CCCCCC)C(=O)OCCCCCC=O ; NCCCCO ; "(C-3)" ; CCCCCCCCC(CCCCCC)C(=O)OCCCCCCN(CCCCO)CCCCCCOC(=O)C(CCCCCC)CCCCCCCC ; "(C-4)"]

Material used for the reduction of the ester aldehyde (C-3) and synthesis of the lipid (C-4), as well as the amount of lipid (C-4) are listed in Table 5. Equiv. and eq are used for equivalent.

TABLE 5
Material used.

| | CAS | MW or | Weight or | | | Molar Ratio | | |
|---|---|---|---|---|---|---|---|---|
| Materials | No | Density | Volume | | mmoles | or ml/g | | Comments |
| (C-3) | | 354.6 | 198.9 | g | 561 | 2.00 | equiv. | Reagent |
| Dichloromethane | 75-09-2 | 1.463 g/mL | 2250 | mL | | 90 | mL/g | Solvent |
| 4-Amino-1-butanol | 13325-10-5 | 89.14 | 25.0 | g | 280.5 | 1.0 | eq | Limiting |
| | | 0.964 g/mL | 25.9 | mL | | | | Reagent |
| Sodium | 56553-60-7 | 211.94 | 237.8 | g | 1122 | 4.0 | eq | Reagent |
| triacetoxyborohydride | | | | | | | | |
| Sodium hydroxide | 1310-73-2 | 1N | 2244 | g | 2244 | 8.0 | eq | Quench |

| | | | |
|---|---|---|---|
| (1N) | 1.04 g/mL | 2158 | mL |

| | | | | |
|---|---|---|---|---|
| (C-4) | 766.3 | 214.9 | 280.5 | Product |

The ester aldehyde (C-3) can be reduced according to the steps listed in Table 6, to form the lipid (C-4).

TABLE 6
Method steps for reduction of ester aldehyde (C-3) and synthesis of cationic lipid (C-4)

| Unit | | |
|---|---|---|
| Op | Description of the steps | Notes/Observations |
| 1. | To reactor charge Dichloromethane | All charges are based on 4-Amino-1-butanol-LR |
| | (1500.0 mL, 60 mL/g) | |
| 2. | Start Agitation | |
| 3. | To reactor charge Sodium | The resultant mixture is a white fluffy slurry. The |
| | Triacetoxyborohydride (237.8 g, | slurry will thin out over the addition of (C-3) |
| | 1122 mmol,) | |
| 4. | To separate reactor charge 4-amino- | The melting point of 4-amino-1-butanol was 16-18° C. |
| | 1-butanol (25.0 g, 25.9 mL, -LR) | |
| 5 | To reactor in UO 4 charge | |
| | Dichloromethane (250 mL, 10 mL/g) | |
| 6. | Charge solution in UO 5 to vessel in | The resultant mixture is still a white fluffy slurry. |
| | UO 3 | The slurry will thin out over the addition of (C-3). |
| | | Order of addtion of step 3 and 6 is not relevant. |
| | | Mild exotherm (~1° C.) noted |
| | | No off-gassing noted |
| 7. | Set reaction temperature to 20° C. | |
| 8 | In separate vessel charge (C-3) | (C-3) is a free flowing clear oil at room temperature. |
| | (198.9 g, 2.00 eq, 561 mmol) | |
| 9. | Charge dichloromethane (500 mL, | (C-3) is carried through crude as a solution from |
| | 20 mL/g) to UO 8. | previous processing (Oxidation). In this TTP, the |
| | | volume of DCM is representative of the volume after |
| | | ioslation from step 2 oxidation. |
| 10. | Charge materials from UO 8 into | Mild off-gassing noted during the addition. |
| | UO 6 at such a rate as to maintain | Reaction is slightly exothermic (~6° C.) |
| | temp of <20 ± 5° C. | White slurry reaction mixture thins out as (C-3) is |
| | | charged |
| 11. | Hold reaction for 120 min | |
| 12. | IPC | Ultra Pure Liquid Chromatography (Charged Aerosol |
| | | Detection) to determine (C-3) NMT 1%. |
| 13. | In a separate vessel, charge Sodium | |
| | Hydroxide (1N) (2244 g, 2158 mL, | |
| | 8.0 equivalents) | |
| 14. | Charge entire contents from vessel in | Minimal exothern with small amount of off-gassing |
| | UO 11 to vessel in UO 13. | noted |
| 15. | Rinse the vessel in UO 11 with | Amount of water rinse is not critical and can be |
| | Water (25 mL, 1 mL/g LR) and | adjusted as needed |
| | transfer to vessel in UO 13 | |
| 16. | Rinse the vessel in UO 11 with | Amount of dichloromethane rinse is not critical and |
| | dichloromethane (25 mL, 1 mL/g | can be adjusted as needed |
| | LR) and transfer to vessel in UO 13 | |
| 17. | Agitate for 30 min | |
| 18. | Turn off agitation | Phase split very rapid resulting in 2 clear phases |
| 19. | Isolate bottom (desired) organic | Discard top Aqueous layer after proper pH |
| | layer | adjustment (pH~6-9) |
| 20. | Charge UO 19 bottom layer | |
| | (organic) to vessel | |
| 21. | Concentrate to ~250 mL volume | Collect 2250 mL of DCM. Distillation can be |
| | | conducted at RT under vacuum. |
| 22. | Obtain crude (C-4) | Solution is ~70% assay and can be stored at −20° C. |

The yield of (C-4), from the above method was 90%.

### Example 4

### Purification Methods for Cationic Lipid (C-4)

A crude cationic lipid (C-4) was dissolved in an n-heptane solution was extracted with a 10% aqueous methanol solution at a pH of 10-11 to remove polar impurities. The extracted n-heptane phase was distilled to a minimum volume to provide a crude C-4 feed for a chromatography step.

Silica gel was charged into a chromatography column. A 90/10 (vol/vol) n-heptane/EtOAc solution was used to condition the column. The crude cationic lipid (C-4) in n-heptane solution was then transferred into the column and rinsed with n-heptane. The silica gel chromatography purification was performed by providing an eluant mixture of n-heptane and ethyl acetate in gradient form with increasing concentration of ethyl acetate. The column was first eluted with 6 column volumes (CV) of a 90/10 (vol/vol) n-heptane/EtOAc solution, followed by 5 CV of an 80/20 (vol/vol) n-heptane solution, and finally with 10 CV of a 70/30 (vol/vol) n-heptane/EtOAc solution or 3 CV of a 50/50 (vol/vol) n-heptane/EtOAc and 3 CV of 100% EtOAc. The eluent from the column was collected in fractions which were analyzed. Fractions which contained minimal or no product were transferred to a waste vessel. Fractions which contained significant product were pooled and concentrated by vacuum distillation to a minimal volume. The concentrate was treated with carbon and then concentrated by vacuum distillation to provide a purified compound. The purified compound was oil-like and in this run approximately 43% more cationic lipid (C-4) was obtained than by a run using the alternative chromatography step below.

In an alternative chromatography step, a slurry of silica in 3 CV isopropyl alcohol (IPA)/7N NH₃ in MeOH was charged into a chromatography column. A 0.5/15/85 (vol/vol) IPA/EtOAc/n-heptane solution was used to condition the column.

The crude cationic lipid (C-4) in n-heptane solution was then transferred into the column and rinsed with n-heptane. The silica chromatography purification was performed by providing an eluant mixture of IPA/EtOAc/n-heptane in gradient form with increasing concentration of ethyl acetate. The column was first eluted with 5 CV of a 0.5/15/85 (vol/vol/vol) IPA/EtOAc/n-heptane solution, followed by 8 CV of 0.5/25/75 (vol/vol/vol) IPA/EtOAc/n-heptane solution. The eluent from the column was collected in fractions which were analyzed. Fractions which contained minimal or no product were transferred to a waste vessel. Fractions which contained significant product were pooled and concentrated by vacuum distillation to a minimal volume. The concentrate was treated with carbon and then concentrated by vacuum distillation to provide a purified compound.

TABLE 7
Primary and alternative column chromatography steps.

| Primary | Alternative |
|---|---|
| Chromatography | Chromatography |
| Steps | Steps |
| Column Packing: | Column Packing: |
| Slurry silica in | Dry charge silica |
| 3CV IPA/7N NH₃ in MeOH | |
| Column Conditioning: | Column Conditioning: |
| 3CV IPA/EtOAc/n-heptane | 1.67CV n-heptane/EtOAc |
| 0.5/15/85 | 90/10 |
| Eluent 1: | Eluent 1: |
| 5CV IPA/EtOAc/n-heptane | 6CV n-heptane/EtOAc |
| 0.5/15/85 | 90/10 |
| Eluent 2: | Eluent 2: |
| 8CV IPA/EtOAc/n-heptane | 5CV n-heptane/EtOAc |
| 0.5/25/75 | 80/20 |
| | Eluent 3: |
| | 10CV n-heptane/EtOAc |
| | 70/30 (or 3CV 50/50 and |
| | 3CV 100% EtOAc) |
| ~42% | ~60% |

### Example 5

### Distillation of Cationic Lipid (C-4) with n-Heptane and Ethanol

The cationic lipid (C-4) formed in Example 3 was distilled with n-heptane and ethanol to obtain (C-4) with purity greater than 97%. Material used for the distillation method and the amount of (C-4) produced is listed in Table 8. The distillation steps are listed in Table 0. Equiv. and eq represent equivalent. The distillation steps below can be the sole purification step employed in the purification of cationic lipid (C-4), or can be used in conjunction with either or both of the extraction and chromatography steps listed above.

TABLE 8
Material used.

| | CAS | MW or | Weight or | | | Molar Ratio | | |
|---|---|---|---|---|---|---|---|---|
| Materials | No | Density | Volume | | mmoles | or ml/g | | Comments |
| (C-4) | | 766.3 | 10 | g | 13.05 | 1.0 | equiv. | Reactant |
| n-Heptane | 142-82-5 | 100.21 | 10 | mL | | 1 | mL/g | Solvent |
| | | 0.684 g/mL | | | | | | |
| Ethanol, | 64-17-5 | 46.07 | 5 | mL | | 0.5 | mL/g | Solvent |

| | |
|---|---|
| (Absolute) | 0.789 g/mL |

| | | | | |
|---|---|---|---|---|
| (C-4) | 766.3 | 214.9 | 13.05 | Product |

TABLE 9
Distillation steps.

| Unit | | |
|---|---|---|
| Op | Description of the Steps | Notes/Observations |
| 1. | A reactor containing (C-4) | In the lab, this initial |
| | (10 g, LR) in n-heptane | distillation (75 torr, |
| | (10 mL, 1 mL/g-LR) was | 0.1 bar) brought |
| | distilled under vacuum | n-heptane levels |
| | Tr = NMT 40° C. | to 0.51% by GC |
| | (target 35° C.) to | headspace analysis |
| | remove n-heptane. | |
| 2. | Absolute ethanol | A ethanol and heptane |
| | (5 mL, 0.5 mL/g-LR) | mixture forms an azeotrope |
| | was added to the reactor | at 34% ethanol at 21° C. |
| | | and 0.1 bar. |
| 3. | The mixture was distilled | In the lab, this distillation |
| | under vacuum at a maximum | (75 torr, 0.1 bar) brought |
| | 40° C. (target 35° C.) | n-heptane levels to NMT 5 ppm |
| | to remove ethanol | n-heptane and 0.3% ethanol by GC |
| | | headspace analysis. Stability |
| | | has been assessed in ethanol at |
| | | 40° C. and at reflux. |
| 4. | Hold mixture under | After the 3 hour hold |
| | vacuum and maximum | under vacuum (75 torr, |
| | 40° C. (target 35° C.) | 0.1 bar), NMT 5 ppm |
| | for 3 hours | ethanol remained by GC |
| | | headspace analysis |
| 5. | IPC to determine | GC headspace to |
| | residual solvent | check for completion |
| | | of removal of ethanol |

Purity of cationic lipid (C-4) obtained after distillation was 97%.

### Example 6

### Formation a Lipid from Reduction of the Ester Aldehyde (C-3) using Hydrogen (H₂)

A portion of the ester aldehyde (C-3) formed in Example 2, was combined with 4-amino-1-butanol and was reduced with hydrogen (H₂) over platinum-carbon(Pt-C) catalyst to form a cationic lipid (C-4), according to the Scheme E4. The crude cationic lipid (C-4) can be purified by employing the extraction, column chromatography, and/or distillation steps described above.

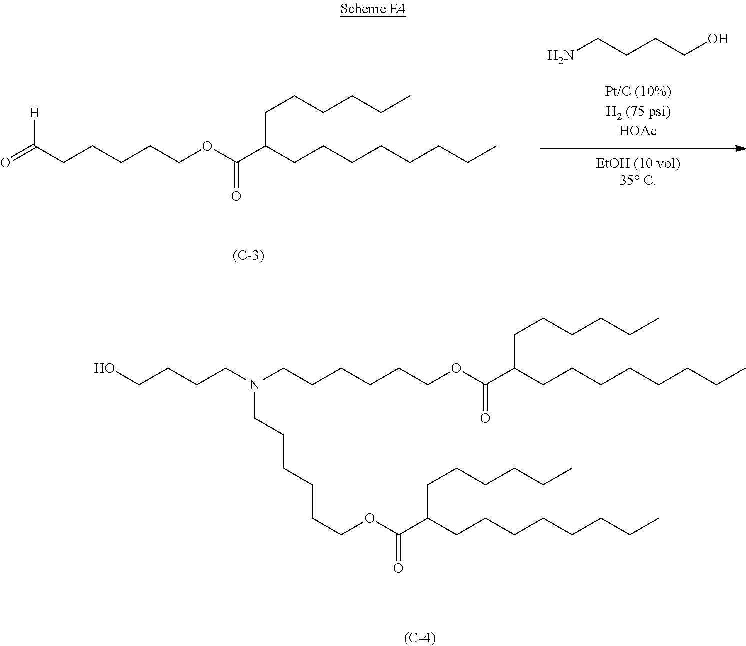 [SMILES: NCCCCO ; CCCCCCCCC(CCCCCC)C(=O)OCCCCCC=O ; "(C-3)" ; CCCCCCCCC(CCCCCC)C(=O)OCCCCCCN(CCCCO)CCCCCCOC(=O)C(CCCCCC)CCCCCCCC ; "(C-4)"]

### Example 7

### Synthesizing a Cationic Lipid (C-7) from Reduction of the Ester Aldehyde (C-3) Using Triacetoxyborohydride

Ester aldehyde (C-3) synthesized in Example 2, was reacted with one equivalent of 4-amino-1-butanol using sodium triacetoxyborohydride (reactive amination conditions) to form a cationic lipid (C-5), according to the Scheme E4. Secondary amine (C-5) was reacted with ester ketone (C-6) under reactive amination conditions to form cationic lipid (C-7).

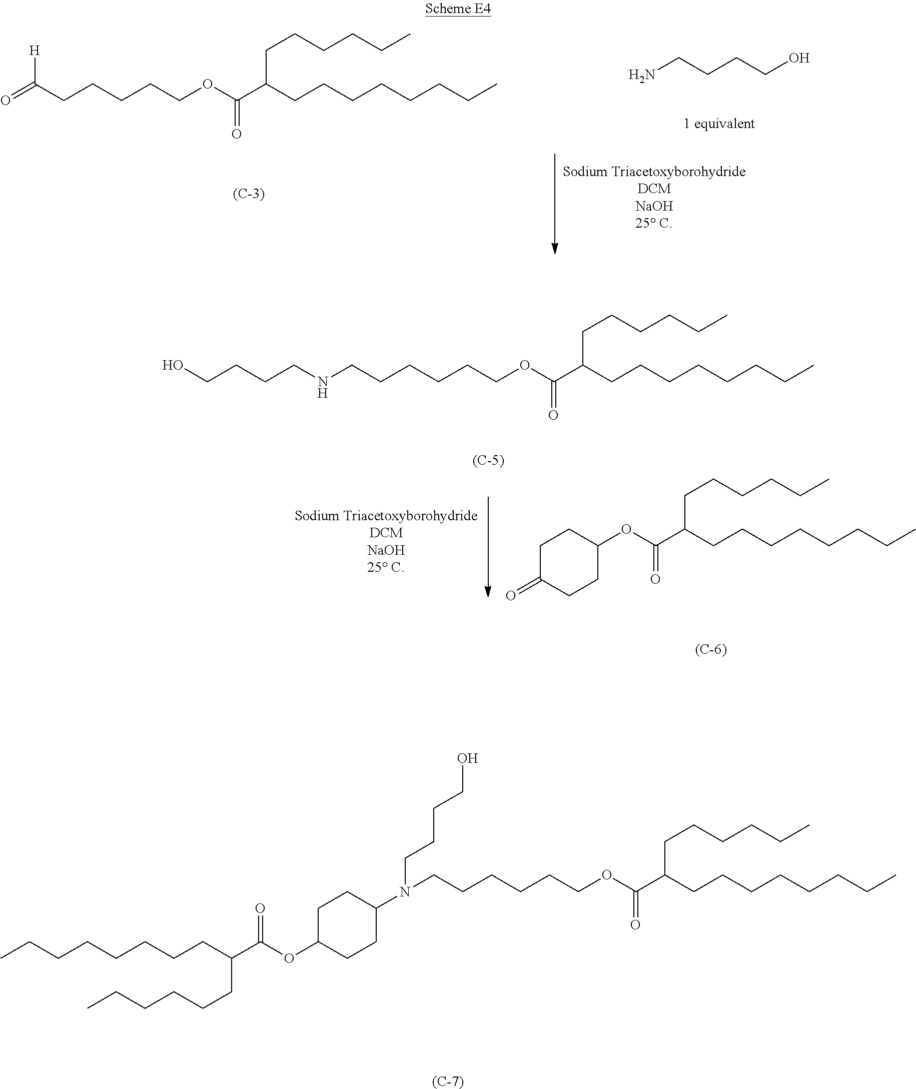

We claim:

1. A method for producing a compound having a chemical formula of Formula I, 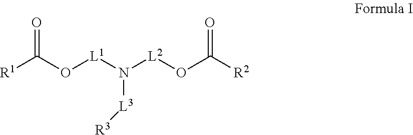
wherein R¹ and R² are independently a i) linear or branched or cyclic, ii) saturated or unsaturated, and iii) substituted or unsubstituted hydrocarbon group comprising 1 to 30 carbon atoms,
R³ is a i) linear or branched or cyclic, ii) saturated or unsaturated, and iii) substituted or unsubstituted hydrocarbon group,
L¹, L² and L³ are independently linkers,
the method comprising:
a) reacting a first acyl chloride having a chemical formula of R¹—(CO)—Cl with a first diol having a chemical formula of HO-L¹—CH₂—OH to form a first ester alcohol having a chemical formula of R¹—C(O)—O-L′—CH₂—OH, and reacting a second acyl chloride having a chemical formula of R²—(CO)—Cl with a second diol having a chemical formula of HO-L′—CH₂—OH to form a second ester alcohol having a chemical formula of R²—C(O)—O-L′—CH₂—OH,
b) oxidizing the first ester alcohol with a first oxidizing agent to form a first ester aldehyde having a chemical formula of R¹—C(O)—O-L¹—CHO, and oxidizing the second ester alcohol with a second oxidizing agent to form a second ester aldehyde having a chemical formula of R²—C(O)—O-L²—CHO; and
c) reducing the first and second ester aldehyde in presence of a reducing agent and an amine having a chemical formula of R³-L³—NH₂, to form the compound of Formula I,
wherein the method does not involve isolation and/or purification by chromatography of the first ester alcohol, the second ester alcohol, the first ester aldehyde, or the second ester aldehyde before forming the compound of Formula I, and/or wherein the method does not involve using a first ester alcohol, second ester alcohol, first ester aldehyde, or second ester aldehyde isolated and/or purified by chromatography.

2. The method of claim 1, wherein the first acyl chloride is formed by reacting a first fatty acid having a chemical formula of R¹—COOH with a first oxychloride, and the second acyl chloride is formed by reacting a second fatty acid having a chemical formula of R²—COOH with a second oxychloride, wherein the first and the second oxychloride are independently thionyl chloride, phosphoryl chloride, oxalyl chloride, or any combinations thereof.

3. The method of claim 2, wherein a first fatty acid solution is contacted with a first oxychloride solution and a second fatty acid solution is contacted with a second oxychloride solution.

4. The method of claim 2, wherein the first fatty acid and the first oxychloride are reacted at a temperature of 15° C. to 30° C., and the second fatty acid and the second oxychloride are reacted at a temperature of 15° C. to 30° C.

5. The method of claim 2, wherein the first fatty acid and the first oxychloride are reacted in presence of dimethylformamide (DMF), and the second fatty acid and the second oxychloride are reacted in presence of DMF.

6. The method of claim 1, wherein the first acyl chloride and the first diol are reacted in presence of a first tertiary amine, and the second acyl chloride and the second diol are reacted in presence of a second tertiary amine.

7. The method of claim 1, wherein the method further comprises,
adding a first base to a first esterification-product mixture comprising the first ester alcohol to form a first biphasic medium, said first biphasic medium comprises i) a first organic medium comprising the first ester alcohol, and ii) a first aqueous medium, and
adding a second base to a second esterification-product mixture comprising the second ester alcohol to form a second biphasic medium, said second biphasic medium comprises i) a second organic medium comprising the second ester alcohol, and ii) a second aqueous medium.

8. The method of claim 1, wherein the oxidation of the first ester alcohol with the first oxidizing agent is catalyzed using a first oxidation catalyst, and the oxidation of the second ester alcohol with the second oxidizing agent is catalyzed using a second oxidation catalyst.

9. The method of claim 1, wherein the method further comprises,
washing a first oxidation-product mixture comprising the first ester aldehyde, and
washing a second oxidation-product mixture comprising the second ester aldehyde,
wherein the first ester aldehyde in the washed first oxidation-product mixture, and the second ester aldehyde in the washed second oxidation-product mixture is reduced in step (c).

10. The method of claim 1, wherein the reducing agent in step (c) comprises hydrogen (H₂).

11. The method of claim 10, wherein the reduction of the first and second ester aldehydes is quenched with a base.

12. The method of claim 1, further comprising at least partially purifying the compound of Formula I by extraction, precipitation, silica gel chromatography, polymer resin chromatography, or a combination thereof.

13. The method of claim 1, wherein the method does not involve isolation and/or purification by chromatography of the first acyl chloride or the second acyl chloride before forming the compound of Formula I, and/or wherein the method does not involve using a first acyl chloride or second acyl chloride isolated and/or purified by chromatography.

14. A salt having the chemical formula of Formula III: 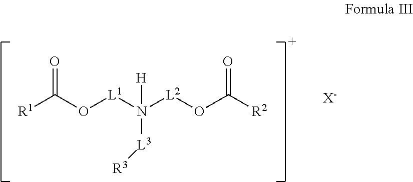
wherein R¹ and R² are independently a i) linear or branched or cyclic, ii) saturated or unsaturated, and iii) substituted or unsubstituted hydrocarbon group comprising 1 to 30 carbon atoms,
R³ is a i′) linear or branched or cyclic, ii′) saturated or unsaturated, and iii′) substituted or unsubstituted hydrocarbon group,
L¹, L² and L³ are independently linkers, and
X⁻ is chloride, bromide, iodide, sulfate, acetate, mesylate, tosylate, (1R)-(—)-10-camphorsulfonate, 1,2-ethanedisulfonate, oxalate, dibenzoyl-L-tartarate, phosphate, L-tartarate, maleate, fumarate, succinate, or malonate.

15. The salt of claim 14, wherein R¹ and R² are independently a branched, saturated, unsubstituted alkyl group comprising 1 to 30 carbons.

16. The salt of claim 14, wherein R¹ and R² are the same.

17. The salt of claim 14, wherein L¹ and L² are the same.

18. The salt of claim 14, wherein i) R¹ and R² are different, and/or ii) L¹ and L² are different.

19. The salt of claim 14, wherein the salt is in a crystallized form.

20. A method for forming a salt of claim 14, the method comprising contacting the compound of Formula I with an acid having a chemical formula of HX.

21. A method of purifying a compound of Formula I, 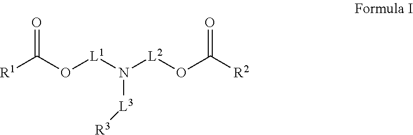
wherein R¹ and R² are independently a i) linear or branched or cyclic, ii) saturated or unsaturated, and iii) substituted or unsubstituted hydrocarbon group comprising 1 to 30 carbon atoms,
R³ is a i) linear or branched or cyclic, ii) saturated or unsaturated, and iii) substituted or unsubstituted hydrocarbon group,
L¹, L² and L³ are independently linkers,
the method comprising purifying by chromatography, and eluting the compound of Formula I with an eluant mixture of n-heptane and ethyl acetate,
wherein the chromatography is silica gel chromatography, polymer resin chromatography, or a combination thereof.

22. The method of claim 21, wherein the silica gel chromatography purification comprises providing the eluant mixture of n-heptane and ethyl acetate in gradient form with increasing concentration of ethyl acetate.
